SEQUENCE LISTING (38 sequences)

SEQ ID NO: 1 - DNA, 8058 bp, Glycine max
  source 1..8058: mol_type = other DNA
atgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggtaccctacgttacctatctatttcccaacttaatttaattactctctcttctattcttatttgccctacgcgtaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcctttggtaagtactcactactcactacattagtttatcatcgtgtttctcttttattaattccatctcataaataacaaaactaaatccatttgattccagttttgcttaggagtaaaactaattcaaatgaatcaatcatattactgaatcatgttagtgtacaaacacgcgcgcattcaccttacctacttttatgtttctcttgtaagctactttatgttacttctgccttaattaataacatcctaaacatttttttggattatttgtctctttaattctattatagtcattttttcatttcgtatagtttatattttagtttttttttattcttagtttatattttaattttttttaattttgaaagtgatatttttagttcctataatttatattttaattatcttttaattcatggtaaataaaatataaaaataattaattataaattcttaactattttttattacaaattatcttgcaataaattaattataaattatttgttaatatttttataattaattataataaaaaatattaattatattttgatgataatgactaaaataaaattaaaatataaaatacaagattaaaaaaaactactttaaaattatatggattaaaatagaattaaaatgtaaattataaagaataaaaatatcattttcttttacctaaatgaaaaaaaaaattaaaatataaattataaggattaaaatattattttcaaataactaaaaagtataaattataaaattataaagaataaataaataattattttctttttgtttcaagatatcctaaacatacgcttcattgttatatatatatataatccaaataaaaccaatatgaccagtttaagctagtgggtaggcgcatattaaaataaaaaacacgaattattattattattattattattattattattattattattattattattattattattatatgttttaaagaactctgtcctcaatcattgtctaatataagttatttgttcatcaacaatcgagatagtaatcaaatcagttagaaatgttgatattgcattttatatcaatgtttatatgcttttattgtttaatctttacatcttcaaatttatttaagatatgaggacaaaataaataaaatgaaaggcatgttgggataagaagaaatttttaattagcaaaaaaatagttatttctaaaataattttttattttattcgattaaatttttttacaacaaaatttagatataaatatctagatttttttgtttgattctttaatttaaatgaaaattttatctcactaatttatgtcaatatttaatgtaaacttttatcatgtaaatgattgatgtaaaattctaattttgattacaataatataaaaaaaagactgtttcgtaggtttgacgtttttaatacattttaaaattttacgtcaaatcaatattagatgcgcaaaattcagattggacgtggatctacacgcacgtagcgttaatgacgagggtgttggcataaataagaaatatacaaaaatgaaagacgtcactatcattatctaaaatggatagtttacaggcttgttgatcataataatttattggattaattagtttttttttagtatttataaaattttaaatttcaagtattagtctctataaaaaaaattatttagtctcattaattttattgctcgtattaatttctgagctaaaattttctcttattgcctattcttagatccttctctctggactaattataaacaataaaaataaacaatacacccaaaaattagaggcagttctttatgtcaattctatacaataaatcctcagtaagctcatagtatactcttttaaaaaaatacaagtaatcactttcgtattctctgaccaaatttactatcaatttaatttttaaataataataattattagagagtatagataaattagctgatgatgcatgtctgggatatatcaattttgtgaatattaaaaataaaataaaattatacattgataaaataagataataattttacatcatcataacaaatcattattatgataagtttattaatttttataataactatcttaataatcatactaataatgaattataactatcttaataattcatatgaacaatgaattataattattagatgtaatgtaaaattattttagattgtcagtacattgattattaaatttttatttttattttttttaaagaaattagaaaattgaaattaaattaaatatgcagcttaggtaataaaaaaattaatatccttggtctcttttttacggaactaaacttataaaagaatgaaatagggatttattcattaacagaataattcaggtcatcattttatgccatactcgaccattaaatgaacataaaaacttagttattgtacatgaaattgaaaatgatcactataattaagaatccaaaattattttcattttttgtttgctcacaagtactcctcttttttacataatcatgaaatatctcagatattttgaacatcatatatataaaattcaagttcattatgcatatgcaataacaaacattaatttttacttgttgaaaatattgcaggttttttgtatgtatattacaggaaatcttgaatcagtatttccagaagaacatcgcaaagaaattcttcgttacacatattatcaccaggtataattattcaatcttaagatacatttgcttaatcaaacataatatagagaaacaaaatgtcaaatttcttgtatcttttaaacttgttgtacttgtttaaccatgaagtttataatctattcattgcaacaataaataattaatattagcccttttgtgctttcatttaattaaagaatgaagacggaggatggggactacacatagagggtcatagcactatgttttgtactgcactgaactatatatgcatgcgaatgcttggagaaggacctaatggaggtcatgacaatgcttgtgctagagcaagaaagtggattcgagatcatggtggtgtaacacatataccttcatggggaaaaacttggctttcggtattattcttttttatatgaatacaaatacaaaaactaatgaaactatgatgtgtactattcatttaaatggatcttgctcttgtagatactcggtgtatttgattggtgcggaagcaacccaatgcccccagagttttggatccttccatcttttcttcctatgcatccaggtagatttaattaattttattttgtcactctgtttggaatgatatatatttaaattggtgaatatacaatatttaatcattaaatctcacttccaccttaactattgtagctaagatgtggtgttactgtcgattggtatacatgcctatgtcttacttatatgggaagaggtttgtgggtccaatcacaccactcatcttacaattaagagaagagttgtttactcaaccttatgaaaaagttaattggaagaaagcgcgtcaccaatgtgcaaaggtgaaagacatatttaattaatcacaatgtgtgtgtgcacatatcttttttgctatgaaaatgatagtcgttcattatgtcaatcacaggaagatctttactatccccatcctttgatacaagacctaatatgggatagtttatacatattcactgaaccgctacttactcgttggcctttcaacaagttgattagagaaaaggcccttcaagtaactatgaaacatattcattatgaagatgagactagtcgatacataaccattggttgtgtggaaaaggtaggtgtcataacaataatgcatattgattttttttatccttatgagtaaaataaactttttattttccttttcaagagggaattcattcaaatatgatactaaaatgcatatattttttttccaggttttatgtatgcttgcttgttgggtggaagatccaaacggagatgctttcaagaagcatcttgcaagggtcccagattacttatgggtttctgaagatggaatgaccatgcaggtatacgcgtgtgggcacgtgctattataaccaattagatacattaattatttaattagtttattgatgcacttatctatataatgcagagttttggtagccaagaatgggatgctggctttgctgttcaagctttgcttgccactaacataattgaagaaattggtcctacgtttgcaaaaggacatgatttcatcaagaagtctcaggtttgcttaatatttttataagtatataaaagaatcccagatcttcttccttctagttatggagcagaaattccctaataaacctaggtttatccagattgtacaattaactaattaagatatttaaaatccctaaatcttatttacagggtgatcgaataaactaaataaaaactatataactaattacttactaattcacctagctacatagtattagttgtaactagtataattatatttttgaattgaaatgcatatttataattgattactaagttggtgtttaactatttattagagtattgaataatatggttgttgtctatatctagtagtaatgaactagcattttcattttgtaggcgaaggataacccttttggagatttttatgaattggcattcttattttataggtgaaggataatccttttggagattttaaaagtatgcatcgtcatatttctaaagggtcttggacattctctgatcaagaccatggatggcaagtttctgattgcactgcagaaggtttaaaggtagccaacaacttaggtcataaacaacttaatctaaagctttgttaatgatcacaacaaatatatgttaatttaactaaaatttttgtgttgtagtgttgtctacttctatcaatgttgccaccagagattgtgggagaaaagatggaacctgaaagattatacgattcagtcaatgtcttgttgtcgcttcaggcaagtaccattaagtttagagaaacgatttatgttatgaaatattttataaaaaattgtctataaattgataaaggttggcaactaatgttgatataatcagctgttttttcaaatccaacaccacctcatttgttcatgatttgtgaaattggttttgtacaatattacaattctcttaaacttatgtccatatccttttaattaaccaatcatcaatcaactaatggccccttttctttgttacttaatatttgatcaataataagttagggttgtacaaaggactttgatcattgatgctttatttacttttactaccaaatttatttttaactaaacaacattttttttattgcagagtaaaaaaggtggtttagcagcatgggagcctgcaggagctcaagagtggttagaagtaagtattaaattaattaataatgcaacacgtatgtatttataaattgataaaaaaaagtacttgattaataaactaattaactttgttggtgattcttagttactcaatcccacagaattttttgcggacattgtagttgaacatgaatatgttgagtgcactggatctgcaatccaagctttagttttgttcaagaagctatatccaggacataggaagaaagagatagaaaatttcattaccaatgcagttcgattccttgaagatacacaaacagctgatggttcatggtatggaaattggggagtttgcttcacttatggctcttggtttgcacttggaggtctagcagctgctggtaagacttacaccaattgtgctgccattcgcaaagccgttaaatttctacttacaacacaaagagaggacggtggatggggagagagttatctttcaagcccaaaaaaggtttgtagctgaatggttattactgaattaccattcttcaacttcattgggaattaatgataatagtaataatgtcttactaaatgaaaataatgcagatatatgtacctctagaaggaagccgatcaaatgttgtacatacagcatgggctcttatgggactaattcatgctggacaggttcgttcttaatttgtgtttgtgtatatttgattataggtctacttcattgtgaattaaatttcaaaatagtttttttttcccctcttcatttttaaattcctatatacaggcggatagagaccccatgcctcttcaccgtgctgcaaagttgctcattaattctcagttggaagagggtgattggccccaacaggtgtatatcaatcctttaaattaaaaaatgtcccgtatttgtgtacatgcttaactaaataaataaataaaaaagaaattaacttatttagcaataaattaaaatcagatcttagaaaaaaagacataggtcgataccatttgggacaagacttcagtgcaacaattcagatgccttgggtgttttaaaatcaaaattaaagtttgacctagctaaagaaattgaaagttgatagatgctattgatgtgaaattctaattttaattggaaaataattccaaaacgtaaaatacaacatttaagttttgtggacaaagttatttctttaaaattttgcttgaactagtattttagtttgtttttataataggattaatttttattttatataattataattcataataaataaatatgttataattaaaatttataatttataataaataaagatatttaaatataaaaattataatttgaatttataataatcaatttaaattgtatggtaggtgttaagtgtattttatattttcaagacaataaatgcatttattattgtatttaataattcagtcgttccataataaatattagaagaaaaataaattctaaaataagtgttatttatactattagtgcatacactttttctctaactgctaatgcttgcaattcatatgcaatactctttgttttattttgattaacctttagagaaaaaaattgacacaatattttagaaaatacaattaataatttagatttcataaaaattgttatgtaatttcctaatcctaatatgtcaatgtatttcatattaattagattatctctcatctatacttttttattttctcactctacatttattgattaatgaagaaaaaaaaagtaattcttcattggttttgtaaaatgacaatcaaaacaatttttttctaaaacatcaattaaaatgaaatggaaggagtaaaatcaaagtctttaaaactttgtcgatacgagaatttcaacttccaatgtaatcaaaacttgatataaaaatattaatatttttttaatttaggaaaatgacagaaaagatttattattaattgtattttgtaagcaaaatgaaaattatgctctagattgagtattgtttcttattgttgtgacctacttttggcaggaaatcacgggagtattcatgaaaaattgcatgttgcattatccaatgtacagagatatttatccaatgtgggctctagctgaatatcgaaggcgggttccattgccttccactgaagtttaa

SEQ ID NO: 2 - DNA, 9399 bp, Glycine max
  source 1..9399: mol_type = other DNA
atgtggaggctgaagatagcagaaggtggaaatgaggcatacatattcagcacaaacaattttgttgggaggcagacatgggagtttgatcctgaagcagggactccagaggaacgagcccaggttgaagcagctcgtaaggatttctaccaccaccgcttcaaggtcaagccctgtgctgacctcctttggcgttttcaggtacgcacgcctacgcctacccttcctctctctctctctctctctctctcacaaatcagcatcaatttcaatctctaattacctcatcctccctaacccctacattatacattatcactccttagaataacattgcattgcatgtctgtcctcttatttttcacttatgcttgtttcccgacacatacgtgtagattctgagagaaaataacttcaaacaaacaatacctagtgtgaagatagaagatggagaggaaataacttaccaaaaagtcacaagcactgtgagaaggggcgcacatcaccttgcggcattgcagaccagtgatggccattggcctgctcaaattgcaggtcctctgttttttcttcctcctttggtaagtcactcaatatctattattattatttatcaaactgatctaattaattacacattgttctaccatctgcttaataaataaatgtcaaaaacgataaacagattcctcctcctcatcatattatacttttctagtttttcattctaatttaagtaaaaaaataaatagtcatttttgtccttaaacatgtagaacgttataaattcatctccaaagaattaaaattcaaattttagtttctaaaagtaaaaaaaatacaacaaatatatttatccattaatttttgttcgttgtcgttaatgaaagagtccatataacacatttaagaacaaatttgtcacaattttatactcatggacgaaaatgattatttacctaaaatataatttaatcttgatcttcttcttgttttttaattgttacaaacataatattacaataaatacttaacaaaataggaaaacaaatataataataagcataatattgattaataagtataatttgtttataccaaaatttctattatgacattagataataatatccaacttgattttgtcattacctaatgttgtcacaataaaaattttacaataaataaattatatttattaatcaatattatgtttattattatgtttgttctaatttattcttgtttttttttgtttttgtaagtagctattataatattatgattgtaaaaattaaattatattttgaataaatagtcatttttgtccctaaatatataaagtgttgacaaattcgtcatcaattaagtgtaacggatgaaagttaatgaatgagtgaatttgttgtatttttttcactttcaataactaaattttgaatttttatttttttaactaatttttgaatactttacacatttaggaaaaaaacgttatttacctcaaataaaataattctaattttataaagggaagaattctaattgcaaaattttcatacgggaaaacgccaacgggcaaataactagacgtgtttccttcttgtcttttatttgatttgattttatctgaaaaagaacaaaagaactcactcaataaaaagtaaatctaggcacgttcatagctccatttgattggagtttaggagaaaaaataattcaaatgaatcatattactgagtcgtgtcagtgtacaaaaacgcgcattcaccttacctacttttatgtttgctttgtacgctactttgttacttttgcttaatataaattagtgacgagatcctaatcatacgcttcattgttttatttttcttcatatatatatatatatatatatatgtgtgtgtgtaaataaacacactaattattaggatttgtctaattgaccctgaataaaaataaaattaaaccatctcatcttaaatcatcaataaaatttagacaattgtcttattttaaaattctatcattaatgttattactatcagatttaggacattatttttaatttctctttcatatcatttatatgtaaaattaaaaatttatattttttaattaaaagttaataattaaaattataaaataataaatcttaataattattttcttaattcactttaggtaaatagttattataaaaaaaatctttatgtaaatagttttcatttattttataacatctaagtataatactttaaatattttccaacattttcattataatttttatttgaaaattatagataaatagtataacttttaattaaaagttatagataaaaagtataattacagcatgtatatttttcctaaggtgaataaagaatataatgattaaaaaataattattaagattttaatatttaataattttattacatactattaatgtattttagtataattttaattaacaattataaagttttaatttacacataaatgatacaaaagaaaaattaaaaatgaaacgttaaatatgatattcatggtattagtgattaaattcaaaataaaataattaccgaaattttattgtttattaaatatttaattatttaatcttatttttacttgggatcaattagataaattcttataattatattaaaaaattttgaagaactttttcctcaatcaatgtctaatattttttattttattcaattattttttttacaacaacattaagatataattttctagatttttttattatttttatgctcgtattaatttctgagttaatttttccttcaaatattgtcttctatggactagttatgaacaattaaattgtttgattcattaaaaaattataatactggataagataaatatttttgtttcacattttatttgacaaagattaaatgataatacaagatgattgacatgacaaaatctataatttttgttactcaaaaaagaaaatcatgaaacaattttttttcccaaatacaagatattaaaaagacacatatatctctattcctatttctttaataataaaaaataataattaaccaacataatcatttaaaaaagctatttttattctaatctctttatgtttttctaactagtaaacaatacactaaattttaggagaaactaaaatccaaaatatgaaattatagtgagataaagaaaacttatttaatcctaatttatttgagaactacaagggtatcagaatgaataagagacaacaaatgagaaaacgtaattatgaaagtcttaaaacgaaaagttttatatgcttattattattattattattattgggaattcaagttatgaactggaaaaatatgaataagaaaattcaatctattgatttggttattatgcagtagctagaattgcagttatacttcggtgatttatattatattaacttgcaacacaattttttactatgcaaattattcagctaaagcaattaaataataatactaaaaacattttttctttaaattgttagtttttatgtcaactctatacaataaatctctttataagtaagctcacattttactcttttaactaatgcaagtaatcactttggtattatctggccaaatttactagcaatttaatttttaaataattattgctatatagtatagttaaattagtggatatatcaattttgtgaaaaaaaatgttttttaggaaaaaaaatgcactgataccataaaataattttatattatcattaatgatgatataaaattattttacattttcaatgtatataactgttaaattcttttcatgtttatttttttaaaagaaattaaattaaatatgcagcttaagtaataaaaaaaaatattatccttattcttttttttatggaactaaacttaaaaaagaatgaaataggaatttcttttattggtaaaaattgaacatgatattaaagggtttataatattcacacattttactcaacaaactgagttaaatcccttgataaataggaattaattcattagtagaatgatttagctaatcattttatgccatactagaccatcattaaatgaacatagaaacttgaagttttgaaactctgttattgtacatgaaattgaaaatgagacactataattaagaattcaaaattattttcattttttgtttgatcacaattcacaagtactcctctttttttacaaaattatgaaatattacacgtattttcatcagcatataaaattcaagtccattatgcatatgcattcacaaacattaatttttacttgttgacaatattgcaggttttctgtatgtatattacagggcatcttgaatcagtttttccagaagaacatcgcaaagaaattctccgttacatatattatcaccaggtatagttattcaattttaggatgcatttgtttaaccaagcataatgtagagatacaaaattgtaacattttcctcggtcaaatttcttgtatcttttaattaacttgttgtatttgcttaatcaggaagtttataatctattcattgcaacaataagtaattaatattatcccttttgttctttcatttaaagaatgaagatggaggatggggactacacatagagggtcatagcactatgttttgtacagcactgaactatatatgcatgcgaattcttggagaaggacctaatggaggtcattataatgcttgtgctagagcaagaaattggattcgagatcatggtggtgtaacacatataccttcatgggggaaaacttggctttctgtattattcttttttatatatgaatataaatactcaaacaaacaaaactaggatgtgtaatattgatttaaatggaccttgctcttgtagatacttggtgtatttgattggtgtggaagcaacccaatgcccccagagttttggatccttccatcgtttcttcctatgcatccgggtatatttgattaactttattttgtcactctttttggaatgatatatatttaaattggtgaatatacaatgtataatcattaaatctcacttttaccttaattattgtagctaagatgtggtgttattgtcgattggtatacatgcccatgtcttatttatatgggaagaggtttgtgggtccaatcacaccactcatcttacaattaagagaagagctattcactcaaccttatgaaaaagttaattggaagaaagcacgtcaccaatatgcaaaggtgaaagacatctttaattaatcagaatgtgtgtatgtacatatcctttttctatgaaaatgacagttgtctattatatcaattacaggaagatctttattatccacatcctttggtacaagatctaatatgggatagtttatacatatttactgagccattgcttactcgttggcctttcaacaagttgattagagaaaaagcccttcaagtaacaatgaagcatattcattacgaagatgagactagtcgatacataaccattggttgtgtggaaaaggtaggtgtcacaacaataatgcatattgattttttttatccttgtgagtaaattaaactttttattttccttttaaagaggaaattcattcaaatatgacactaacatgcatttttttctaggttttatgtatgcttgcttgttgggtggaagatccaaacggagatgctttcaagaagcatcttgcaaggatcccagattacttatgggtttctgaagatggaatgaccatgcaagtacgtgtgtgtgggcgcatgctattataaccaatgggatatattaattaattcgttaattaattgattgtttcacttatctatataatgcagagttttggtagtcaagaatgggatgctggctttgctgttcaagcttttcttgccactaacctaattgaagaaattggtcctacacttgcaaaaggacatgacttcatcaagaagtctcaggtttgcttaatatttttataagtatatattaaaaatcatatttctctttccttttagttatggagtgtaaattccctagtaaacctaggtttatccaaattgtacaactcactagtttagatatttaaaatccctaaatcttatttacaaggtgatctaataaactaaataattaatttacctagctaaatagtattagttataactagtataattatattatttgaattgaaatgcatatttataattgattactaaattagtgtttgttagactattgaacaatatgtttgttgtttttatctagtaataatgaactggcattcttattttataggtaaaagataatcctcttggagattttaaaagtatgtatcgtcatatttctaaaggatcttggacattctctgatcaagatcatggatggcaagtttctgattgcactgcagaaagtttaaaggtacgtaactaacaacttaggtcataaacaacttaatctgaagctttattaatgatcgcaacaaatatatgttaatttaactaaacttcttgtgttgtagtgttgtctacttctatcaatgttgccaccagagattgtgggagaaaagatggaacctgaaaggttgtacgattcagtcaatgtcttgttgtcgcttcaggcaagtaccattaaattttgagaaacgatttatgttatgaaatattatataaaaaaattgtctataaatggattaagggctgacaattaatgatgatatgatgggctcttttctcaaattcaacaccacgtcatttgttcaagatttgggaaattggttttgtacaatattacaattcgcttaaacttatggccatatgcttttaattaatcaatcatcaatcaactaatggccttccctttttattacttaatatttggtcaataagaagttggggttgtacaaaggactttgattagtgatgctttatttacttttactaccaaatttacctttaactaaaaacattttgtttgttattgcagagtaaaaaaggtggtttagctgcatgggagcttgcaggagctcaagagtggttagaagtaagtattacattaattaataatgcaatatgtatgtactcataaaatgataaaaaatagtacttgtttgataaactaattaactttgttgttgattctcagttactcaatcccacagaattttttgcggacattgtagttgaacatgaatatgttgagtgcactggatctgcaatccaagctttagttttgttcaagaatctatatccaggacataggaagaaagagatagaaaatttcattgccaatgcagttcgattccttgaagatacacaaacagctgatggttcatggtatggaaactggggagtttgcttcacttatggctcttggtttgcacttggcggtctagcagctgctggcaagacttacatcaattgtgctgccattcgcaaagccgttaaatttctactttcaacacagagggaagacggtggatggggagagagctatctttcaagcccaaaaaaggtttgtagttgtatggttattactgaattactattcttcaacttcattgggaattaatgataataataataataataatgttgtacaaaataaaaataatgcagatatatgtacctctagaaggaagtcgatcaaatgttgtacatacagcatgggctcttatgggactaattcatgctggacaggtttgttagtgtttatatttgtttatataggtccacttcattgtgaattaaatttcaaaaatacttacttttttgtggtaactatcatcaaatatgataatatgttcttcatcttcatttttaaattcctatatacaggcggatagagaccctaagcctcttcatcgagctgcaaagttgctcattaattctcagttggaagagggtgattggccccagcaggtgcatatcaatcctttaataccgaaaatgtatcgtatttgtgtacatgcttaactcaaaaaggcaaaaaaataattgacttatttagtgataaattaaaatgagatcttaaaaataagacataggtcactgacatttgggacaagacttcagtgcaataatttagatgccttcagtgttttaaaatcaaatttaaagtttaacctagcaatagaaattgaaagttggtacatgttattgatgtaaaattctagttttaattggaaaataattccaaaacataaaatccatcatttaagttttgtcaacaaagttatcagtttaaaatttttggtagaactagtgttttagtttgtgttttcataaaattaatgtttattttatataattataattcataatacataaatattttataattatactttataataaataaagatatttaaatatataaattataatttgaatttataataaataatttaaattgtatggtaggtgttaagtgcattttatattttcatcaagaaaataaatgtattgactattttagtataaagtaatagaataatttatatggggatgtatataattactagagtttaataactatgtattgataatataagttttatataatttttcagtcatacatcacctatgattcttgaggtaattattataaaagtaaaaaaaacttaaacatataagataatataaaattatttatactattaatgcatagcttttttttctaactactaatacttgcaattcatatacaacactccctttgttttattatgattggcatttaatgttttgatacaatactttagaaaatataatcaatatgataggtttcataaaaattgttatgtaatttccgaatatgttactgtctttcatattaattaattagattatctctcagttattcttttttattttctcactctacaattattgattaaggatataatggaaaataaattaattctttattgattttgtaaaatgacgatcaaaataaaatatgttttttgaaacatcaatcaaaatgaaatggaaggagtacaatcgaagtctttaaaacttcatgtcgatacgataatttgatcgtgtatttcaacttccaatgtaatcaaaacatgatatacaaatattaatattttatttttttataatttatgaaatgacagaaagatttattattaattgtattttgtaagcaaaatgaaaatgatgctctagattgagtttaatttctaattgttgtttgtgacctacttttggcaggaaatcacaggagtattcatgaaaaattgcatgttgcattatccaatgtacagagatatttatccaatgtgggctctagctgaatatcgtaggcgggttccattgccttccactgaagtttaa

SEQ ID NO: 3 - DNA, 23654 bp, Glycine max
  source 1..23654: mol_type = other DNA
atgtggaggctgaagatagcagatggtggaaaagacccatacattttcagcacaaacaattttgtaggaagacagacatgggagtttgatcctgaggcaggaactccagaggaacgagctcaagttgaagcagctcgtcaaaatttttataacaatcgcttcaaggtcaaggcatgtggtgatctcctttggcgttttcaggtaccaaacagtacctaacaaataattacttttctttcaagtttgcattttacatctctagtgtctttcttttttcatgcatgaacactttcttgttgtgacatcaagccattggccatcactggtctttcttttatccaaaacaacaacaaaaaaagtcctttcataaacatcaaatttaatcctcaactcattctccttaatttaataataaagaacatatatactcatgcacagagatgcttctattttttaaggaataaataatcatatttatactattcaatagcaatttaactttaaagtgagttttgtaaaaagacaagttaatcatgctattcactatttagttagagcatcacgatctcatgcttggaaccatgtcctcgacaatatccctatatccctacttgtattgcttctttgatcggtaatggaatgccataaatttatttcccctacagaacaattcaactcttagattcaatttctttttttaaataaatcacgtccacaaaatttaatataattttaaaaataactgtattctcatatataatttttttaatataaataaacttcaattgtaaaatccaatatctataaatatttaaatttaatatacataatcattataataatatttcattatttaatatctgtgctattttatttttgccatctataaatatgactaattttcacttatgtttcccgccacgtgtagattctgagagaaaaaaacttcaaacaatcaatacctagtgtgaagatagaagatggagaagaaataacatacgaaaaagtcataagcacgttgagaagagccgcacaccacctatcagcattgcagaccagtgatggccattggcctgcacaaattgcaggtcctctgttttttctgcctcctttggtaagtactaattaattactcaatattaatataatttttattttatttaaccatttaacgaaatgtttatccaactttccatctggttaagaaaaatctgtgcatttagaatcacggaataagaaaaagtcaaaaatgataaattgatttttcatcatattacatttatgaatttttgcttcaaaattaaataaaatagttataatttttatgactaaaaatttggaaagggatatagcaattaaatgtagttgcatgaacttgtcaaaactagacatgtttactcctttgtcttttgtttacgtttactttgaaatatccttattttagtaaatagggtgtaaattgatttgttttttcaaatgtcattttttaaatactaattattcttctatttataatctaattcaccaaaaaaatttaatttagttgataattataaatttgctttaaatttatactctctatatctctaattatgagacataattgactcatttatgcttattaaaaaattaaaaaattatagttatagtatttctccaaatttataattaactttattgagaacctactcaatattcatcttctcccatctccataggagaggtaaagaataatttagagtattttaatataaaaaaataattaatgtacaaaagaaatgaaatgaagtcttataataaggacacaaaaatgtgttgactattccctctagtctcaaatataaggaaaaaaacatatttacttggtcttaaatataagaaaattttaattaacttaaccttttttaattttactatctataaaatactattcatttaattaaaatgttagtttcaatgtctcttttcttatccttgataccaagtttcaataaaagataagttaaaaaaatatttttaattaaaattgatacaattaaatataattaattaacattattaatcaatgtgtcatatttttttcttatatttgagatcagaaaaaatatgtgttatagatatgaatcaaaagggtatactttttcaaacatacaattgtcattaattattagtgtttccctctcttataaatagcaagtcaaatataactttaatcttttcttattcaattaaaatatttgagtttaaaaataattaatgaaagaaatataatgaattggatcttttaaaaaaacaaataaatttgaaaagtttgttctggtaaataggaagctagtaggttacaagtgtagtattaattgcttatgaatatcaaatttagaatccaaagagttaacttttaaatatcattatttcaaatgttttgtttaaataggttacaagtgtagtattagttgcttatgagttatgagtatatgtggaagaaaattaatgaaacttaaaatttgtaatgcatcaattattttaaaatatcaagaaaaaataaaatatcaataattttaatacaaaaaataatatccgataaattaaaaataaaataaagccaaaaaaataaacaatttagataatattattgtgctgaaattcagtcaataaaaagtaaatttaggcacgttcacaggtccatttgaatggagtttaggagtaataataattcgaataaatcatatttaattgattttaattttttttatctaatagctcactataatttcaccaagcttgtattagaatatctgaattaagcacaagggatgcccaattcaaacacaaattacagatgagattaggcctcctaacacaaggcataaaaacaaaccagtaaatgacattaatgcactatcctgcctatacaagggataaccacttcgacataacaaacggtgctttgcaatacaaactcatatgttatatcgatgtaattaggggtgttgaaaatggaatcaaattaattttaaatttttaaattaaaaaaatttaactaaaaacagtttaaataaaaaaattgtatatttttgtatagtttgactcgatttttagattttgttatcaaaatcaaaccaaattgcacattacaattagatggatttatttaatggatgtgtagcatattgtagataatattatttaaattttataatggttatagtatgtgatttatagtatatttttttctataataattttataataatatataatttttgttgtggtaattaataataagtattgaataatttattatattttcttttcttatatgatcaagaatcatattttgttttcatatgttaaaaaagatatatcaatatgttaaaaaaatagtgttaaatattaagtgatgcataaattactattatattgtaaaaataattggtaatatcattttataataatgataaataaatgtgaaaataattattttcaactaaaatatgttaaataaaatttaatataaaataaaattcatttttaaaataatatagcataataattatataagtttaattgatagattttaaattattttaataattaaattaaaaaataaaactatcaaatcaaactataaaagattagttaaattcgatcagaatttgatgttatatttaaactaaattaaagtaaaccgcatttttattttatatttaatttgaatattttttatttaaaaattgaattaaatcgcatcataaacacccctaatgtaatataccaaaccaccaaacaatatttttccatataatatcaccaagattaacgaagttggttggcccatgataaaataaagcatgaattatatatgtattgaagaactttatccttaatcattatctttttgtaatattagctatttgctcataaataatcaatatagtaatcaaatcaattcaaatgtatgaagaaaatctaactcaattgttgggactcagcctaccgaaactactcttgatgcaacattaaggatttttcactatttaacgttattctgattattaccttcacccgttaacctaccctaaccatgatccctgatgtgaaagagcctaaagtacctaatttcactcctaatcccttaagagctacgtcaaataatttgctttaagaataaagatgcataaataaggctaaataatatcattctatcccttgacatagattacctagatgttctttgcaagttcttaggagataaacattttccaatgcataaaccctataacaccacatgagcatgggtgatcaaaccataaacatgatattaagcgcaaaaaatagacaataatatcaaaatagcattaaatagatggttaaaatcattacatcaagaatgtttgattgttgagctcccaacaatgggtggtttaccctctctttatcatgagagacttactaatacaaaaaagaaacaataggtgtggaagaaaagggaggaaagggtccccaagtgagtgggttctcccttttctctcgtaccctaactccttatttggtcccaaaaaaattgttttctggtttcttccttttttcctttaaaacgcactacaatcatgttttttgtcattgatcgtgcgctaagcctgcatgtgcgcattgagcgcacatgcaaagtccagctggcataagtgaccgcgcgctatgccgccagatgcgcactaagcctggcctccaagttggcttctcgcgctgagcggttgttgacgcactgagcgtgctactccaattctccaacccccttcttaaccttctattgtgtctctacaaaaaaacatactaccaaagatactataaatttatcaatttaagcatttattagataaaaactcagaatatgtcaaaatcctaatgtttaacacaaaaagaaggaatacaaggtggaaaaatcagataattacaacatttacgtatatatagaaattatcaaactcccccaaatttaaagttttgtttgtcctcaagcaaaagtaagcaaaactacactaagtcgggttaatgacaatgtttagaatgtaaccaaccacaataactgagataattgcatacttttcaaacaatttcaacttccaaccctcaagttcacacttgtcatggttttcatgatggaatcatgcaaagaatgcactcagagattgaaataggttggcaagtatggcaagtatcaaagaaagatcaattatgtttcactcctcacaaggctagtgtttctttgaagcacacaagtgttttaagttatgtgtgtttcatttaaactgactgaagttataattcttatatttacagatcatctcattcaatgttattgcacacacacaatatggatcactaagaattttattaggcttgtaacgtggttgggctaacaagaaatcatggttttttttttttattcaaacacttaggttttaggagagcactctctcatttcctatctacttgaaataccacttttttcaaaaccaccaacctttattgtcatgccacacttaatagcacacaaggattccttggtaaacatttgcttccagctcttatttctccatattttgtttttttattagttttttttagactaccacagaatgtcattctggttttttgtataattgttactatatatcgctgcaacacaagttttccaaggcactcccccaaatttaggacaaatttgtcttgatccataacagtgctttcctataacctaagacacggtgcacaacagtagtatctgcattcatctcaaggttcaatgacaaaattcattcacatttagactctaaagggtgcaagggattcaatcattcattaatagtaattatttggctaagtgactgaaattaaaataaacaacaaggccttgatcatatccacatcacgtatgcactcaagcaattcaagaatcatgcaaaatcgagaaaatgaaatcagtcgttctcttgcaattaaggtgcacacaactcacacgttaatgtgaagcatttagtttttcatccataatttcctgtcatgcaagctaaccaactcactcatgcgcatttaatttacaaaaccatcactcaacagcatgtgctcactcaaaaattgaaaatttcacaacattaataaccagtacgcaatgtctcagtcatgcaatcaattcaaccaaacatgcctcaaaaagtgttaactatcagagttacccaaaatgcatcatcatgtctttcatgaaaaattaaacataataaagaattcaaatttaaaaagtaacaaaaaatgaaataaggcatgagggagtggtgtgctaagcacacatgtgcgctcttagtgctccataaaacaaaacaaacataaatgcacagtgggcttagtgcataagtgcgcgctaagtctacagcgtaggagacacaccaaattaaaattacaagaaattttgattttttttgggctgaccccaaacttgtgcgctaagcctaaacttgcacactaaacctgaacttgcgcgctaagcccaaataattaaattgcaactccagagatattattggacttaacacgccatatggggcttagcgagttttgcaggaggaaatttcctgcaaaatccgcttagcgagcaccataggcttagcaaaaatgcatgcctcgcttagcctgggcctgcactcttagcccaattcgagatattacaaaacaaaggggttttggggcttagcgcgacatgcaggcttagctagttgcccattcgtgcttagtctgcacttgcgcgcttagcccaaatacaaaatatttcaaaacagggagtcctttgggcttagcgtgacatgcaggcttagcacaactatcaaaacatcatgtgcgcctgcacgcttagcccaattcgagatattacaaaacaaaggggtttttgggcttagcgcaacatgcaggcttagctagttgcccatgcgtgcttagtctacacttgcgcgcttagcccaaatacaaaatatttcaaaacaaaggggcttagcgtgacgtgcaggctcagcgcaactatcaaaacatccattatgtgctaagcgcacacacatgcactgagcgcataacataatcaaacaacaaataataacaaacatctgcactcaaattaactaacacaaatattcaaagagtcatgggtgatcaaaccgtaaacatgatattaagtacaaaaaagagacaataatatcaaaatagcattaaatagatagttataattattacattaagagtgtttggttgttgagctgctaacaatgggtggtttagtctctcattgacatgagaggcttactaatacaaaaaggaaacaaaaggtgcagaagagaatgaaagaaaatggaggaaagggtccccaagtgagtggattctccctcttctcttgtgccctagctccttatttggtcccaaatgctggttaagccgccaaagacgcgctaagcctggcttccaagttggctcctcacgctaattggttgctgacacactaagcaagctgctctaattcttcaaccctcttccaggccttctgttgtgtctctacaaaaagaatataccaccaaaaatactataaatttactagtttgagcacctactagataaaaactcagaagatgtcaaaatcttaatgtttaacacaaaaagaagcaataaaagaggaataaattagataatttttgtgtgatttaattaccacatttatgtatacatagcaattatcaataactaataatatatgtacttggtaattttttaggcatgtgaaacaatctcacttacttcctcaaatttcccaaagtcttgcaacatcaaattaatgcagtgagcagcacaaggagaccaatgtaacttaggaaactctttttccaataacctaccaacaacaacataatttgaaacattatcggttagaatgtttgaacaacatcttttgcaagacactcttcaatggctttgtcataattttctacaatttgcttattttgcaagacactcttcaagctaggttgagctccaggagtttttctaggcatgaaacagtcatctacatgaggagtgttcttccccttttgagactaggaaaaaactagtggttttggagttcctcttaattccttacctattctcatagggtcttcatccatttcaaatgtgttactgtcctaagatttttcctgcacttttcttttctttctttttttttcaaactgttcaagtagttgcttcattttatgttggacatcagtaggaactctcttgcaggcctttacttgggcttccagccaagtgagccttaaacctattaataatacctcccttaacccgtttatcacaataaatacacatcattgtcacttttgtagcaccctcattaatttgtttacaatgtccccaagctggatcatttttctttctaacattctgagaagtatttggagtggatgatgcttgagataatggggcttcaacagataacatcttcaagtctaattgcaaacattagcaatcaaagaaattccaaataactaagggtatgtttggtttgcattttcattttctattttcatttcttgttttcattttctaaaaactgcttttattttcaaaagattagaattctgaaaacatgtttggtttgacttcttattatctgctttcaagaaataaaaacactgaaaatacgttttcaaaaggaaatgtatttttagatttgtttaaaattacattacttgccaccgcatttttattttacccaaaatgaggttcctagtttcaactgaaaacactaaaaatgatattttattgtttccagtttttggtttgtttgggaaaatattttcattgaaaatgaaaacaaaaatccaaccaaacacattttcatcatcattttctatttccagtgaaaatgaaaacagaaaacaaccaaaccaaacaccccctaaaattgtaaaaagactaagttaattatgagttagtgatcatatatggtattgaattccaaataagtacattaacttagattctcatacatcaatagctattactatatattagacaattattatcaaatatcaataacaatgagtgaatgactacaagaatagacacaatgaactaaactaagtgaatttactaatctcagattctcaacaaaattaactcaaatagcagttactacctactacttactagagtctacaactctacaagaatagataatagtggcattataaataagcataagtgcattacatatttacattaatgaatttacaaacatttgcaattgaaaaatgtcaaatatcaattgaattataatttataaataagcataacaaaacacgagttttttccccaaaacaataacaccaatgcataaaataataaaagcataagcaaaaaaataaagttaaaactaggtggaggagaaatggagaactcaccaccgaaaatgagacccaggtgcgacgacaagttgcaaagtgctgacccaagtgcacggcgtcgagggagttcgtggcttcatgcgacgatttgcagacccagttggcaagtgtgtggtgaccaacaaattgcagaccaagtgcagtggcgagctcaagcatgaagaatgaagatgaaaataaggggtctctggctagttcgagtaagactgagatttttagggatagtgacaaacactcaatctcaaatgcattattttgttctttttggattgataatgagacatcaaaacaatgttgtacaagtggtccaatttttttttatgtgcacactgccagttcgggtttgtaaaaccggtcgggtcattgggttccctcaaaactggtcaggtttggccgagtcatcccaagccacatgcatgaatagaccaatagtgaaaccgacttggttagatcactggatcccggttggacctgtctgactagcaaggccgaaccagttttcataacattgttgttactgacccaagaaaattttacacacgaacataatcaaacatggattatttcgctaaataaatcattttcctacatgtgaatgcaatgagaacttatacctttgcaccaaccctttgggttaattaatttggttgagtttgataagttgtgaattcatagtttaaattaatttaattacttcaatttaaattaaaaagatataacttacgtgtagctttttagatgtttttcatattgtttttcactaagatatgaatttatagacctaatctatctctaatgcaaacttcccttacataaattattgagatgaagctttaagagtacaataggaaaaataattattaatttctttatgtcaattctactcaataaacattgttttagtgtactcatattttactcttttgaaaaataaaagtaatcacttttggtcttctctgaccaaatttataatatatttatagcatgtttggttttgtggtgttatgtgatattttacattaagttagaactaaataattatagttttcacttttttttcttcactagaattaattgatgtgattttatgctgttctaaaagggtgtctaaacatattgttaatttttaaattattattattgcatatagttatattagttgaagtcaaataataaaaaaatagtttatgatgcatgtttagaatatatcaaattgtgaaacaattaattttaatttctttaaaagtatttaaataaactaaaattaatagaatattaagtaataaattagtaaaaagttactaacttcattctattttttatggagctatttttttttggtttatggtactaatattttattaaagattgaaatatggatttattcaataataaaacaatagagtttgtcaatttaagatatactagaccattaaatgaacatagaaattcgaagtttggaaactttggtatttgtacataacattgaatatgggacactgattaagaataaaaaaattcaattaatgtttggtcataagtagtttctttttttataaaataattatgaaatattaactaggacataaatttaaacatcatatacgaaactggactcaaatccattaaggtaattaaagattaatcatcgataaaattcaactaaaacaacattaatcgatattaatttttacttattcaaaacattgcaggttttttgtatgtacattacagggcatcttgatttggtattcccagaagagtatcgaaaagagattctccgttacatatactatcaccaggtatacctattcaatttcaagattcattttcccaatcaagcatgattatatatatatatatagagagagagagagagagagagagagagagagttgtaacattttttgttttcaaaattcttgtatcatttaattaacttgttgatgtatatgcgtgaagtttaaaatttgttcattgcaacaaagaataattaatattgtccattttgttcttcaatttaaagaatgaagatggaggatggggactacacatagagggtcatagcaccatgtttggtactacactaaactatatatgtatgcgaattcttggagaagggcctaatggaggtcatgaaaatgcatgtgctagaggaaaaaagtggattcatgatcatggtggtgtaacacacatcccttcatgggggaaaacttggctttcggtatcacattcagttttacataaatataaatacacaaactagctagtatgtatggtttaaatggatcctactcttgtagatacttggtgtatttgattggtgtggaagcaacccaatgcccccagagttttggattattccttcgtttcttcctatgcatccaggttgagtttattttattttattttgtcactctttattcaacaatatatgtttaaattgttgaatatacgatgtttaatcattgaatctcattttcaccataattattgtagctaaaatgtggtgttattgtcgattggtatatatgcctatgtcttacttatatgggaaaaggtttgtgggtccaatcacaccactcatcttacaattgagagaagagctctttactcagccttatgaaaaagttaattggaaaaaagtgcgtcatcaatgtgcaaaggtgagaaaactaatatttaatcaatcgcaatgtgtgtatgtacacattctctttatcatgaaactgataattgttcattctatctatcacaggaagatctttattatcctcattctttgatacaagacctagtatgggacagtctatacatgtttactgagccactacttacttgttggcctttcaacaaactaattagagaaaaggcccttcaagtaacaatgaaccatattcattacgaagatgagaatagtcgatacataactattgggtgtgtggaaaaggtaggtgtcataacaataatgcattaatttgattgtttagccttatgagaaaaattagtttcctcttcaagaaggaaactcattcaactatgctaaacatgtatatgtttttcaggttctatgtatgcttgcttgttgggttgaagatccacagggagatgcctttaagaagcatcttgcaagggtctcagattacttatgggtttctgaagatggaatgaccatgcaagtacgtctatgtgtgttattataaccattaggatgtattagttaattagttaattaattaatcacttacgtatatgatgtagagttttggtagtcaagaatgggatgctggttttgcagttcaagctttgcttgcaactaagctaattgacgaaattggccattcacttgcaaaagggcatgatttcatcaagaagtctcaggtgtgtttaatttttatgataagtacataaaagaatcataattctcctataaggaaggtaaattccccagtgaatatagtttattcaaattatgcaattcattaatcaaccaaaaaatttattacttactaactaatttgtctgactaacaatattaattataactagtataattatagtattctaattgcaatgcatattaatttataattgactattaagttagtgttcattagattacttaagagtatgtttgttagctttacatagtagtaatgaattgacattcttatttcaaaggtgagagacaacccttcaggagattttaagagtatgtatcgtcatattactaaagggtcttggacattctcagatcaagaccatggatggcaagtttctgattgcactgcagaaggtttaaaggtatctgacaacttagctcgtgaaaaatcaattaatttaataataagctttatcaattacaaatatatgttaatataactaattaaagttcttctgtgttttagtgttgtcttcttctatcaaagttgtcaccagagattgtgggagaaaaagtgaaacctgaaagattttatgattcagtcaatatcttgttgtcacttcaggcaagtaccattaaatttggatagaaaaactaaacacagagatttggaaggaaactcatcaaacacaaagaagacaaataaaaatttgctttgatgtatattacatttaaacataacactataagtgttacagtaagttgcttaaatttaaaatctataaaagtctattattgaacaaagcattagaacaatcatgactcattttacaaatatgcaaaagttaacatcgttaacctacaactagctattttttaacacaagcaaattttacaattcttacaagaaatttaacctacaactagttatttcttaacacaagcaacttttaccattcttgcaagaaatttggaattacaaaaacatcaaaaaatttagatgtagtttttggaaaaatctcaatttccaacaaatttaaagaaatgtttgttggaattttaggggatccttataaaataccaatataaccactaggaacacattatgttagattatcaagaagagttttaggaatatctggatccataatgattgatcaaataaatacagtgaaatctgaatctgtaggtgatttatgatctcttaggatccattacggaatgtcgtagagacaatagataattggctaacaacgtaacatgtggcttcacggttcttcctcaaccgaaacttctttattcctcaataagacattcataattcttcagatggggagaagagaaaccatgtctgatgactcggtatattggggaccatagcattcttatagcgtgttaacgtaatagggttccgattatcccctaatggaccaacactgacatttgctcatttaagtccatatcatctgatttagttttctaatgggctcacttaatttgatcacataaaataaaactcactaatgacatatatctaatatgattgtcttataatattatccaacattaattattagaatagaaaattccaacaatctcccacttggtccacatattgtaacgattatatcaaaatccttaagcgtgcatccatatgctatttacctttagacttccaccttaataacttggtccatctcatgtatcaataatggaatcactacgactttcatcactacaacaaatgtaactaaacctcaatgaccaccacatcaatacactcagtcaatgacataggtcaatatggataagcagtatgaaaattacatgtaatgtgatctcagtcatgtgtatttccaattggtccaaactttattctttatagagatcaatccaaacaaaacataaatattgcaaacaaaacaagcttataatgaaatgataaacttcaactttatttctgcagaaaatccaaacaacaaaatgtttgtaaaccataagatatagaacataagcaagaatttcactaaactaaggtactatcaagagcagtatgctcatgaaaaaactttaagtatcaaacctttagtaagtagatctgctagtatggaatgagtccctatatgttctatacaaatttgtattttctgaattctttatttaacaaccaaatattttatattatcaacaaacttttacttggttgaatccttaataagaccgttaagatattatctcaataaacacctgatggcttcttaatgttggcaacaacaagcaagctagttacaataatttagctgtaataaattatagtatgatgtctcataaaaaaatattaactccaccaatatggttaaataaggatccttatgtggagtgattgctattaagatattccataaaaatcaaaattagaatacccaatgatctctaaacttctagactttcgatatgaaaatatgtagtttcttgttctcttcaattagcacattatgtgttttactactttctagtgatgcatacccagattactcatatatcaccttaggactcccactaaaaattattttaggacaataataaaaacaattaaaaaataaacttaagcatacatgataaattgcaatataattgttttgcaacaataaaccccaacacaatgccggaaaaaaaaaacactcgaattcaaaccaaatatccaatatgacaatatttgaatttaccgacaattaatatgtgttgtgtcaaaaaaatagggttcataaaaacaaagcaattaaggtataataagaatcaaataatgttaaatcccaataatcaaacagtaatggtggctctgataccacttgttggaattttagaggatccttataaaataccaatataaccaccaagaacatattacgttagattatcaagaagaattttaggaatacttgaatgcataatgattgatcaaacaaacacagcagaatctcaatccttaggtgatttctgatctatgtactcttcttagtatttgttacgaaatacgatagaaacaacagataaccagctaacagtgtaactggtggcttcatgattcttcctcaactggaacttttttatttctcaataggaccttcgtaactctttagatggggaaaaagaaacaatgtgtgacggctcgatatattgaggaccatagtcttcttatagcgtattaacctaatagggttctcattatcccctaataggttaacactgacatctactcatttaagtttatatcatctgatttaattgtctaacgggctcacttaatttaatcacataaaataaagcacactaatgataaataactaatataattatcttataatattagcccacattaattattggaataaaaaatttcaacaatattaatgctatgaaatattttatagaaatttgctacacatttcttatagcaaccaattaatgttgatgatggactccttttctcaaatccaacatcgcatcatttgttcaagattggtgaaatttgttctgtaccatgttgcaatattcacttaaattcatggccatgtgcttttaattaataaatcaatcaactaattgcccttctctttgttacttgatcttttatcaatgggaaagtagggattgtgcaaaagactttggtaattgatttttttacttttactaattgattttcatttgacacaactacatttttttattattgtagagtaaaaaaggtggtatagcagcatgggaaccaataggagctcaagaatggttggaagtaagtattatattaattaataatgcaatatgtacatattcatcggttgataaaaatccattcatcaattaataaactaatcaattttttcattaattttcagttactcaatcccactgaattttttgaggacattgtaattgagcatgaatatgttgagtgcactggatctgcaattcaagctttagttttgttccagaagctatatccagagcataggaagacagagattaagaatttcattgtcaatgcagttcaattccttgaagatacacaaacaaccaatggttcatggtatggatgttggggagtttgctacacttatggctcttggtttgcacttggtggtctagcagctgccggtaagacttacactaattgtaatgccattcgcaaggctgtgaaatttctacttacaacacagagagaggatggtgggtggggagagagttatctttcaagcccaaaaaaggtttgtaattgaattgttgttactaaataattactctttaaccttagttgatgataataataattgtagtaatgtcttttttaatgaaaacaatgcagatatacatacctcttgaaggaagtcgatcaaacgttgtacaaacagcatgggctcttatgggtctaatttatgctggacaggtttgctttttatgtgtgcgtgtttgtgtgattataggttcacttcatcgtgatttaaattttaaaatgttttctttttgttgttgtaactacaatcaaatcttgtaataagttttttccctttatttttaaatttctatgtacagtcagagagagaccttactcctcttcatcaagctgcaaagttgctcattaattcccagttggaagaaggtgattggccccaacaggtgcatatcaacctttgatattgataatagatcttatttaataaacttatttagtgataaattaaataggatcttaaaatataagacaccaaacgtgaagattactagggataacgcttatgtgcaataattgagataccttaggtgtttccaaattaaaattaaagtttcacctcgctaaaagacattgaaaattggtacatgttaccaatgtgaaattataattttaattgcaaaataatccaaaaacatacaatacaacgcctaagttttttcgacaagttatctctttaaaatttcggttgagttaatatactaatctataccttgtacaagataaatatttattttatataattaaatttaatataaataataaatatatattataattaaagttcataataaattaattaatatatttcaatatatgaattatattttacatttatcataaacaatttaaattatattataaggttattttataacttttggtaatttcttttagaaaaaactattaaaattaattcaatttagaggcagggagaaaagatgtttattgaaagagatacaaaattactaagataaaatgtacaaataaagaagaaataaagaagaaaaaagtgtagtttgagatggtcaagagaggaaagttttggctagttgcaaggataatgcatgaattgtacattcaaaacacagaaaagagaatgccgagaatgtttaattgagaatctcactcttcaaattgtgtagtctaatttataatataggaataaattaaatcaataaaaaaaggaaaatacaaaatttaaatgtcaacaaagttgaaatgtatacaacaataaacttgcaattcctatgtaatacaatctaattatttaccatttcaatttgatgttgatgtgaaaacattaaatgctcataaaggagagagagagaagatgacgctgtttacacacgtgagtgatcaaatgactgcatgaatcatattgtagcttacgtgaagaagcttttatacaatggaagaaaaagaaggtagtgcgaaagctggttgttataccagaaaaacagaaaacactaactgcttgtaactaataactgaaaatgctaacagactcatgctactcatatacttgatatgtgctaagagctcccctcaagttgggaatggatattcagcattcccatcttggaacagagatcttgaaacaaagagggtgagagtggcttcgtaaaaatgtcagctagctacaattgcgaggagataggaagcagtttgagggatccttgattcaccttatcacgcacgatatgacaatcgatttctatatgtttggttcgttcgtggaagacgggattacaagctatttgaatagcggacttattatcatagtatagggttgtgggctgaaggaaggagacctgaaagtcttgcagaaggtacgtgagccactgaagctcacatgttgtagacgccaaggcccgatattccgcctctgaggagcttcttgaaacagtaggttgtttcttcgaacgccaagaaataaaggatgagccgagatataccgagaagccggtgatggaacgccttgtatcgcggcaaccggcccaatcggagtcactgaaagctttgagttgcagagggccgtcggccgcaagaaagatgcccaaaccaggtgagcccttgagatatcataagacacgaaatgctgcttgtgagtgagctgaagtagggtgagccatatattggctgagttgttgaacggcatgagtgatatcgggacgagtgttggtgaggtaaatgagtcttccaatcagccagcgatatgaggaagaagaagtttctgatagaggagtgcccatcaaggctgttaatcgggtggagtagtccatgggagttgaagcgggtcgagaggcaagcatgcctgagccggataagatatccaagacgtacttacgttgagataagtgaatgcccttgttattgcgagcaacttcaagaccgagaaagaacttcaaggtgccaaggtccttgatgctgaaagtttgatcaaagaggtttgtgatatgcgtgatggtgtcgagattgttacctgctatgattatatcatcaacgtaaacgagcacaacagtgatatcactgttattgaagtggagaaaaagagaatggtctgctgcagattggcgaaagtcgtgagagatgaggaaagaagagaatcgtgtgaaccattggcggcttgcttgtttcaaaccatataatgaacgttgaagtcggaaaactagattgtgatcatgcacctgaagacctgggggaagagtcatgtgtacctcttcgtcaaggtcgccatgaagaaatgcattattgacgtcgagtttcctaagatgtcaattgtgaagggcagcaagagcgagaagcaatctcacagtggtgagcttagccacgggagagaatgtatcaagaaagtccaatccttctagttgtgtgtatcctttggctacgagacgcgccttgtatcgttcaacagtaccatctgctttatatttgatcttatatacccagcgacattcgatagctgtcttgtgtggtggtagtggtgtaagggtccatgtttgattctgttgaagagcttgtatttctgcctgcatagccttaatccaacagtcaaattgggaagcctcatcataagatttgggttcagtgtttaccgatattgacatcacgaagtgtttatgggaaggtgaaaggcgggagtaggataggacagaatgtagaggatacctaactccacgcgtattggtatcagcttgtgaagtgagtgcatggtagtaatcttggagataagtgggtgccttttttagtctcgtggaacatcatagaaaaggagaaggtatatgtgaggatgagggattgtcattgggtggaatggggtgttcaaagctgtcgggattattggagaatgggagagatgatatagaagtaggaggggtggatgattctggagtatcagggaagagaggaaagtggtcttcatagaaaacaatgttactggatgtggttatgctatgggaatggagatcatatactaaatatcctttggtgtttggtttgaaacctatgaagatgcatgggtgggcacggcggtcgagcttttgtctgtgagatttgagagtgcttatgtagcataggcaaccaaaaacccaaaggttcgagatatcacaagggcgtgtgtatagctgttcatagggagatttgttatggaggaaaggagtagggatgcagttgatcaaatatgttgcatgaagtaatgcaaaacaccaaaaattaagaggaagatttgcttgaaataatagagatcgagtaacatgcaagaggtgttggtgctttcgttcaacgatgccgttttgctcaggggtctctatacaagtagtttgatgtatgattcccctttgagcataaaaatcattcataagaaactcagcaccattatcactacgtatgattttaatggttttattaaattgcatttgagcatatgtaacgaatgtgataatgagttttcgagcctcagcttttgtttgcattagataaacccatgtaaagcgagaatgatcgtctacaatggttaaaaaataacgatgcccatgcatagaaacctttgaacagggttcccatatatctatatgtatgagatcaaaaatgcttaatgcatgtgaagtgcttgatgaaaaggatgatcgtttatgctttgcatagtgacacgtagtacatacaaagttcttattgttattatgcaaagaaggataataagtttgcatgacttgcagcctttctatagagggatgacccatgcgaaaatgccataaatctatagggatgacattacattttgggtgaataatggtggagcatatggttcgtgagtgtgatggttttaagatgaactggtaaagcccatgactcaattcaattgtaccaatcttcgctttggtgttggtatcctgcagaacacaagtattagaggaaaagattaactcatagttgtttgtggaaataagtttggatatggatatgagattaaagctaaaagaaggtatgtatagaacatcgttcaatataatggaaggggtgaggtgtactgttcctaagtgagtagcatgaacttcgtgtccatttggtaacttaaccataataggattaacttgatgatatgagtgaaagttggccaaagaagaggagacgtggtcagtggctcctgaatctaatatccaggaggtagattctgtggttttgagagaaagaatcgtacctggtgaggtgttagtggagcatgaggatatggatgcaacctgggattgggtagggattgagcctcccgaagatggctgttgtattaaggcgagcaaagcttgatactgctcaggtgaaaaacgaaccaaatcatgagactcttgtcgttgcatttgatcactcgtagcttttccttctatgatgttatttactactttgttgttgccaaacttgtatcctggggggtagccatgcttcttataacacacatcaatggtgtgtcccatcttcccacaatgggtacacgttttcccgtttctgttgtaggttttgcttcttccttcatagcttgtgggtaccccatgtttcttgtaacaaacactttcggtgtgaccaattcgttcgcagaattcacaagtaatcttgacggcattaatggaaacattttcttttgattcaatactgagatttgaaaaggagttgatacctgatagctgacattcttgttgggctacataggagaagatctttggtatggtgggcatgggttccatgagcaacacgtgagatctcacattgttgtattgctcgtttaatcctcgtaggaattgcatggctcggtcttctaatttccgttgggcaatgatggtaaggactgagcatgagcacttgatggtgcaagagcaagtggggtcaggtctgaagttctcaatttcatcccatatgatacgaagctttgtgaagtactccgtgacggaaagggttccttgtttcattgatgaagcttcttgttgaaggtccgagattctcaagaggtctccttgcgcatatcgtgacttcaagtcattccatatttcctcagctttgttcatccacaagatgctctgcctgatcgcgaccgatactgaatgcactatccacgatactgccatattattgcagcgactccacgcactgtaggttctatcagttctcagaggttctggtgcgttcccatttatgaattctactttattcttggcgcttagtgctgtgaccatggacctgctccatgagtgatagttgcttgagtctaaaactggtgatacgagagccactgctggattctcgcttggatggagatagaggtaactctccatatttggtgttgattcgttcatagtgaataacgtgaagagaaagaagaaataaagcttgggtatgaatgcgcagcagagctctttatgaaaagagctctgataccatgtgaaaacattaaatgctcataaaggagagagagaagatgatgctgtttacacacgtgagtgatcaaatgactgcatgaatcatattgtagcttaagtgaagaagcttttatacaatggaagaaagagaaggtaatgcgaaagccggttgttataccagaaaaacagaaaacgctaagtgcttgtaactaataactgaaaatgctaacagactcatgctactcatatacttgatatgtgctgagagttgatgtgagagttggatcgcgtgcttcagtttcctatgtaagaaaaattgaaatatagatttcaatattttgattattttttaaagatctatccttaattgtattttgtaagcaatatgaagaatgatgctccggattcagtttttctatttctaattattattttttgtgacttaattgatctggtaggaaatcactggagtattcttgaaaaactgcatgatgcattacccaatgtatagaaatatttttccaatgtgggctctagctgaatatcgtaggcgagttccattgccatccactgaaggttaa

SEQ ID NO: 4 - DNA, 6533 bp, Glycine max
  source 1..6533: mol_type = other DNA
atgtacattacaggacatcttgattcagtattccccgaagagtataaaaaagaaattcttcgttacatgtactatcaccaggtatacgtatcaattcacgacatattaccaatcaagcatgattaacttgtaacattttcttttttaaaagttgtatttgctaaatcatgaagttttaaaaaattcattgcaacaataatgaataactaatattgtccattttacgctttcatttatagaatgaagatggaggatggggactacacattgagggtcacagcaacatgttttgtactgcactaaactatatatgtatgcgagttcttggagaaggccctaatggaggtcatgacaatgcttgtgctagagcaagaaaatggattcatgatcatggtggtgtaacacacataccatcatggggaaaaacttggctttcggtattattcattttaatataaatataaatacacaaactaacaaaactagtatgtatgttactaatttaatggccaactattgtagattcttggggtatatgattggtatggaagcaaaccagtgcccccagagttttgggttcttccttcatttcttcctatgcatccatgtatagtaaattttattttattttgtcactctttatagaatgatatatatttaaattgttaaatatgcaatgtttaatcattgaatctcattttcaccataattattgtagctaaaatctggtgttactgtcgattgacatatctacctatgtcttacttatatgggaagaggtttgtgggtccaatcacaccacttatcttacaattgagagaagagctctttactcaaccgtatgaaaaggttaattggaagaaagcacgtaaccaatgtgcaaaggtgagagccattctttaatcaatcagaatgtgtacatgtatgtaaatattttatctctttatcacaaaactgaaaaatcaattctatctatcacaggaagatcgttatcatcctcattctttgatacaagacctggtatgggatagtttatacatatttattgagccactacttactcggtggcctttcaacaagctagtaagagaaaaggcccttcaggtaacaatgaagcatatcctttacgaagatgaaaatagtcgatacataaccattgggtgtgtggaaaaggtagttatatcacaacaataatgcatttatattttttagccttatgagaaaaagatgaaccttttattttcctcttcaaaatgaaattcattcaaatatgtaactaacatgtatatattttaggctttatatatgcttgcttgttgggtggaagatccaaatggagatgctttcaagaagcatcttgcaagggttccagattacttatgggtttctgaagatggaatggccatgcaggtacgtgtgtgtgttattataaccaatgggatatattccttaattaattaatgcacttgcctatataatgtagagttttggtagccaaggatgggatgctagtttggttattcaagctttgcttgccactaacctaatggaggaaattggtcctacacttgcaaaagcacatgatttcatcaagaagactcaggtttgcttaatttttattacaagcatataaaaaaatcataattctccttctagttaaggagtgtaaattccccaatgaacctagtttatttaagttatacaattcactaattggaaatattcaaaatatacttgggtcttatttatagagtgatttaacctattaagtaatcaatttactaactaatttaccagctaactaataatattaattaaaactagcataactatattattcgaattaaaatgcatatttaaattggacgacgaatttagtgttcattagattattgattaatatgtaatttgctagctttacatagcagtaatgaactaacactcttatttcacaggtgaaggatgaccctttaggagattttaagagtatgtatcgtcatatttctaaagggtcttggacattctctgatcaagatcagggatggcaagtttctgattgcacggcagaaactttgaatgtaactaacttagctcataaacaacttaatttgttataagctttatcaatcactacaaatatatgttaatttagcttttttttttttagtgttgtctacttctatccatattgccatcagagattgtaggagaaaagatggaacctcaaaggttatatgattcagtcaatatcctattgtcacttcaagtaagtaacattaaattttggtagaaaattgaacacaaaacgatttggaagaaaacaacatcaaacaaaaaatatagaaacataaaattgttacgatatgaatagcatacactaaaagtgtaattgcaagttgcttaaatacaaaagctagctattaaaattaaagcccttgattggacacagaaatagaacaatcattactcattttaggtttcttttaaccctataagggtaatttagaaaaatatatcaaaatggataattttaaaaaattattcaaatttaaataaattattggttcgagtgttactagatttgatcctcctaatccggattttgagttcaagttttagataattcttttattattatacaagttaaattaatttgagtggtcatcaatgcacaccttttttttaaaaggatcatgagtggccattattgtacacaataagtattctttcattttattttttttttaatttttgaagttagttttataaaatttataccacttttctttctttttctttattaacaaattcatttaccatcgaatgcgtagacgaactggcacgggattgtcctaacttaatcaaaggttttaagtttgagtttttggcatacatctacattaaatactaggagggagagttttttccttagtgataacaaaattattttccgtgaaagatacattaggtctataaaaaaataaaatgaattcattgcaaacataaagattcaccaattcacttaataaatgaaaaacaaaaattaatagaagtcatgggtgtgagcatgacttaatatatgcatacacgacttcaattaataaataaaaaattataaatgtagcttttgtaattttctttaatttagtttttattattttttaaaaaaatataaaagaaattggtgtttcatatgacaacttcagtaaatgaaataaaataataaaaaataaagcaaccttgggttttacggcatgaactctgtaaaaaaactgttaaagatatttagaataaaaaataaaaacaaataaaagttgtaaaagtaaaacacaacttcgtaaaaaaaaatgaaaatcttgtttacttagtaaatccaaaagtcacataatagtatacaacttccatttagaagtcatgtttcataaacatgatttaccccaatttaaataatttttaaaaaaatatttactcattttgataatatttccaaaaattacccctccgaggtaaaaaatatttactcatttagaaatttgcaaatgttaacaccattagcctacaactagttatttcttacacaagcaacttttaatataatttgaggaaagatctcaatttctgacaatttagataaatacttaatattatgaaatatgttatagtaagttgtctataaattaatggctaatagctgacatatgttgatatgatagactccctgtctcaaatccaatgccacatcatttattcaagattcgtgaaatttgtttggtaccatattgtatttctcctaaattcatggtcatatgcttttaattaattaataaatcaatcaattgatggtccttctctttgtttaatcttttatcattgggaagttggggttagcgcagacgacattaataattgatgtttgatttacttttactactagaatatttacttttgatgcaactgtggttcttttttctcttaattattgcagagtagaaaaggtggtttagcagcatgggaaccatcaggaactcaagaatggttggaagtaagtattatataagttaataatgcatgcaacaagtacatattcattaaccgataaaaaaaagtcatccattaattaataaactaatcaattttgtcattactttttagatactcaatcccacagaatttttagggaacattgtggttgaacatgattatgctgagtgcactggatctgctattcaagctttagttttgtttaagaagctatatccaaggcatagggagaaagagataaagaattccattgccaatgcagttcaattccttgaagatacacaaacagctaatggttcatggtatggaagttggggagtttgctacatttatggcacttggtttgcacttcgtggtcttgcagcggcgggtaaaacttacactaattgtgctgccattcgcaaaggcgttaaatttctactttcaacacagaaagaggatggtgggtggggagaaagttatctttcatgcccaaaacaggtttgtaattgaatggccatggtaaatacttaatgttcttcactttcactaggaattaataataatagtagtaataatgtcttactaaatgaaaacaattcagatatatgtacctcttgaaggaagtcgatcaacagttgtgcacactgcttgggctcttttgggtctaattcttgctggacaggtttgcttttgatatgtgtgtgtgtgtgtgattataggtccgctacatccttatttaaatttcaaaatgcttaaaattttgttctaagtaccataaatcttataataagtccaccccggccccctattcatctttaaattcctatgtataggcagagagagatactacttctcttcaccgtgctgcaaaattgctcatcaattctcagttgaaagagggtgattggcctcaacaggtacgtatcaatcctttgatattgataatgtctcttatttaataaacttattgagcaataaatttagttgaactagtaattatgcgttgtacaaaataaatgttttttttatattattaaaattcataataaataatttttaaatatataaattatagcataattaaaaatcacaataaaatactaaatatctttatatacatacatatatatatatatatatatatatatatatatatatatatatatatatatatatatatatatattatagattgggtcactatgtttatagttcctaaaaaagggtctaaatgtttaccagcaaaaaaaaaatttggaactaaaaacaaatcgtaaaaaggttaggaactaaacattagaaaacctttttagatttaaaaagaaaaatttaaattatattgtcaggttatttttaattataggtaatttctttattaaaaatcaatttagaagtagagataaaaaaggactttttaaaaaaaaaaagatggacgactaataagtaaagagtttaatgctatgtaaaactcttttacacagttatttaaatacaaattatcgtttgaattattttaagataattattttaaaagttaacaaatttattatacatggatgattgtgtaaattttttcactataccaatgtataacccttcttcttttaagtaaaatatacatgtaaaggaactaaagatagtagcttggaatagtcaaaagataaaaggtttggctagctgaaagaattaatagtgtctgaaatacacattgaaagcagaaaaaagagagaatgttgaaaaatgaaaatgtttaatggagagttttactattcaaattatatattggagtttataatataggagtagattgtagtaaagaaaaaagtaggccaatacaaaatttaaaggtcaacgagattgaactttatacaatatataccttcaattcctaacaaatttttcctccgatcttaaatataagagaaaaattatttcatgctaattattcatattaaattacatcaatttcaattaaaagttaattttttttcctaagttatccttcgttacaattagaatttggtattatgaataaaaaagaataattgaaactaaaattatatttcaattagattaggaatatttataacctaaaattagttgagatttttttagatttgagaccaacaaaatgatattttttttcttcttatatctaagatgggaaggagtatatatttcaattcgatgttgatatgagagttggattatgtgttttaacttcctatgtaaacaaaacttcaaacacaaactttaatattttgattatttttaatttacaaaaatgatgcaaaatgtctattattaattttttttaagcaatgattcttcagattaatgatttttttttctatttataattgttgtttatgacttacttctactaggaactcactggagtattcaatagaacgtgcatgctgcattacccaatgtacagaaatatttatccaatgtgggctctagctgaatatcgtaggttggttcccttgccatccactgaagtttaa

SEQ ID NO: 5 - DNA, 10995 bp, Glycine max
  source 1..10995: mol_type = other DNA
tttctaagggaaaaaaatttcagacaaacaataactagtgtgaagattcaagatgatgaagagattacaaatgagaaaactaccaacacactgaagagggctattcattacttgtcagcactacaaactagtaatggctattggcctgctcaatttagtggtcctctctttccaactgctctatttgtaagttcaacttattcaaaatatattgtttataataaaattatatttaaattagttattaaataaaaaaataatcataaaattaatatatcttcaataaattaatttattaaacttaaagcatttccaatactattaatcataagatattcactttaggtatcttatattaaaaatgatttaatgagttctcagactatcacgtcatatttagacattctacgtataaatttattttaaagataatacaattgaacaattcatatttttttatttttttatttgtacttaatctttttgaaattaaaatattaatttgatgattttatttgatacaataaaataattttttatttgtaagcaatcttatctttaagataaaaagtttacttttaagaaatcctttcatgtcaagtaaacttgttccaatgaaacaaaaaaaaaattatatcttaaaataagatactcataccaaagagttctaactcgcttgattaagtagtgtagataacttattataaatattttatatttgagttcaatttctactattaaaatgaaaaaaaaaaagatactcataaatatacattgcttaaatatgctcttacaaattgcttctttatttttaagatgaactgattggtcaaacttattttaaagggatacagttttgtcaatatttacttattcatgaaaaactcaaattaaagtcacttaaaaatgataaagtgtgtcggttgctaaatttaatttgaataagtaatacagtttttatggctgaaattttgaagaaaaaaattattatttcaaattgatttacatacccatcgttgcccaattaacgtatgattttaacatggtaaccaaaaaaatatcaataattatgtatgattcatggatttttttatttacaaatctcggtcaattttggttgaacaaatctatactgtttatggttgtgaactaaattaatatttaattgtaagaatggacaaaataatagctagtatggacttattcattaataaaatgattaaattcttccttatcaaattaagaaataaacatataaatttaatttttattgattgttatgagataaaaaaataataaatataattttctaattagagagtccaaattatgttcaataaccaacgtatcattagtttaagtaaaattgagtttgatttcatttaacaagattttaagtttgaatcttgcaaatgaaaaattatatttggaggaataaattctatgaaaagtgactaatcaaattttttgatagaaattattcattaataaaattgataaatactttatactaatatatattcacctaaacaaattattttcaatattattttaatataatcaattaaattccttaataatcatgaaattaagatctcctataatgtgaaaaaaaaggaaaagggacaaaaatacattacatgagctcaaatatgtgtaatcataatttttgaactacacgttaaccaaattatcaactggttgatctaaaagaactttaaacaatattgacaaattaatctttacaaaattttgatagttatatggaagtgtttaatatttgcaatagaaaattaggatttattttgtattcaaaatatacaacatagaaaaaaatatatagatttgtgtatatggaaacaacaaattttttcttcaacagtgtgcagacttgtatttacactgagactaatatctaaattctaaagagcaaactcccttcctactttttaggtctaaaaaacagtcttgaatttttctagtttgtgtctagtatcctttgcactcagttagatgcttctatttataatgctaacgagatatggatttaagaatttttttttaaaagagagggtttatttaaaataattttatatctttttatttttatatttaaaaataaaaattatctcatatatttttatcttaagaagtttcctgatataagacatcttattcttattttatgaataacttctacatttataaaataatactctttgtttatattaattatattcttggtgctagcaaaatatataataatattccactaaaatatttatttatatatataatttaataaatgagatctattaacctttaccaaataaagaccattacatatatattgatctatccagattattgatatcatattcacaataatcttacgatcaagaacaatttagattaaaattataaaagacttatttctcattatcataatctttatcatggtaacaagtttctaattttaatcaatgacttgtcaaattaataatttaataaaaaaataaatatgataataaaataaaaaataattctttactaaaattcataacatgataaattggatcttagacatacatatttatcccaacatattctctaaaagttgaagtttgtaatgctaacacactatttaacattatttttacttacttaaaatactccaggtcatgtgtatgtacattacaggacatcttgactctatatttccagaagagtatcgcaaagagattcttcgttacatttattttcaccaggtacacttgttaaatttcaatatgataccatgttgaaggagaaaagaaagaaaattatatacaatgttaaatttttaccattttttttcttttgtaagaaatattgtagcttcatttatttataactagctcatcattttgcttaaccataaaatttgaaatttttcatcacaacaataagcaattaatactaaaatttactttcatttgaagaatgaagatggaggttggggactaaatatagatggtcacagcaccatgttttctactacactcaattacatatgcatgcgtattttgggagaaggaccgaatggaggtcataacaatgcttgtgctaaagcaagaaaatggattcatgatcatggtagtgtaacacatataccttcatggggaaaattttggctttcggtaagctatttctattttaagttaattaatacacaaatatgcatgctagctactaatttaaacaatcttataggtacttggtgtagttgattggtgtggaagtaaccctttgcccccagagttttggatccttcctactttttccccaatgcatccaggttgaatgatttaattttgatttgttactcattgtgtaacaatacataaatgagtttgagtatacaattaatacttaattattgattgatgaatatttattatatctcatttttaccttaaacaatgcagctaaaatgtggtgctattgtcgattgatatatatgcccatgtcttacttatatggaaaaaaatttgtgggttcaataacgccattagtcttaaacttgagagaagaactctttactcaaccttacgatgaaaatagttggaagaaagcacgtcacaaatgtgcaaaggtacgagacattggatccttaaaatatgtgcatttttaaattcacaaaaaatatgaaatataatttataatatttagaacaatagaataaaatgcttagttagttcaattaaaacaaaaatagaaaaaatagaaaaaaaagacattgttgtaaagaagacacataaacataattaaattttaaagaaacacatgtcactctaatatatatataaccattataaaaggaatgaactctaattaactcaaatataatcaaaattaactaattttttagttaattaagaaagcttaatatcatttgattctactaattatgtttaaaaaataaactatttttctaacatgttttttttttattggaatatgatatcaagaataaaaaagaatcattgaaaatagaatatcaattaattaattgaaggaattttttggaatagtaccattaaagtaattaaatttgacttatatatgagtttaaacttgttctttgttgctaataattgagtccggatgggttaaatatgtataaattaactacaacgagaatgcatatgtaaaatgtttatgacaataaatgttaattgtttgttttatatattgcaggaagatctttactatcctcatccttggatacaagatctaatatgggatagtttgtatttgttcgttgagccactccttacacgttggcctttgaacaagttgatcagagaaaaggcacttcaagtaacaatgaagcatattcattatgaaaatgaaaatagtcgatatttagatatggcgtgcgtggaaaaggtatgctacataaatactaaaacatattattttccaatatatgttaattgaaatgttactaacatttttatttttttcttaattgcacttttttatcctttactatagtcatttcttgaatttggtcccctcatcttttttttttaatctatccctccattatatctaattatgtaaatttggattatctattaacttgtcatccaatatttaaaagaaatgttgatgcaacaataaggtattgaggtggatatgtataatgtagtagagtatcacatcaacatttttgttaaatattagatatcaaattaatatattgacaagagttactccattaaaatagtggaggagggggaaaattcacaaaaaattaggaaaattaaatttgagaaatgacaataataggagacaaaaaatgtaattaagcatttagtttttctacaaagagaattttgtcaaacatgccactaacatattggtattttagagtttatgtatgcttgcttgttgggtggaagatccaaatggggatgctttcaagaagcatcttgcacggattccagattatttttgggtttcagaagatggaatgatcgtgcaggtaaatatattggattgtcagaattcttagtgcatacattgtaatacttgaatataaattaatcaataccatattataacaataataataagtaaccataaatgaaatataataatgataattagtaaggtacattgactaatgcatttgtttttataatgcagcccgtaattggtagtcaagcatgggatataagcttcattgttcaagctctacttgctacagatctaattgaggaatttggtcccacacttgcgaaagcacatgacttcatcaagaaatctcaggtcaacttaagtttcacaataaaaatacgataagatcacaattttttaaatagttttcaatagattaacaacaatttgtgtacgttatatctatcaagcaaaaatgaattgacgttctagtatctcagctcacagacaatcttccaggagattttaagagtatgtatcgtcatatttctaaaggagcatggaccttttctcacaaagacgatggatggcaactttctgattccactgcagaatgtttaaaggtactaactacatggatctcaaacatcaatattaataaagttcatgaacaacatatacttgttaactaactaattacttatcccattttcagtgttgtctgcttttatcaatgatgccacaagagattgtgggggaaaaaatggaacctgaaaaattatatgattccgtcgatttcatattgtcacttcaagtgagtattattaaatccatgttaatatgctttcaattgattcattaaataatcaattgtccttctccttaattactattttttttatatcctatatacttttgtactatggatatgaaaaataaaaaattgtccaacacattagttaattgatcacctattgggatattggtaagagtactaaattgttcaagtaacaaagttataagttaagtcatttccacaataacttgtgcaatacttgggtaacaataataaattatgcataccatgaaggtttgagtatcaaagtaaccaaaataaagatagatgtagaaaattaaagagtttaaagtaagtaaagtggtttgagaattttgttgaacacttggcagacacagaaaaatgggttgtcactattggatgatattgtcgggatttaatcgttatacttttctaacatataattattaattacttctattaatttaagactatgttatcattaatccaaaaattattctaaccttaataccttaagcgaaaaaactttaatctcttgattccattatcaatcccttaactagtctaatcaagcaacttgcattaaaaataattttaatgatgaccaataacctttatgttatccctagatgtaatggttatcatttgtcttcttcaattcacaattcaattgaaatttccaaaacaattgaataaataggatcatccaattaggtgattgaattaagaacaagcaaaaagcacaaaaaaagaacaataaaaacctgtactaaataagaaaaataacaattacatagaagtagtagtctaattacattaaaccccaatgggaatgaatgcaactacccatacccatagaaattgcgtaaagaacaatcagagtaaagtctttaagcatagatgcctccacactatctttctctaacctttggaatgctttttgtatctttgagtgtatttgtttcacggattaaaagtgcattcccagaaatgtacacatgagaatgtaatatgctcatgtttgttataagtttttaaaaagtattctcaaattattgattccggtgaatatttataactttctttttaccatgatttcttatatttctatttttatgagtaatgagtaacttatattttcacgagaatatgagaagttattgtctaatatacatgtatccatggttatcaaactctcgagttaactcactaactcttacgagtccacttgtcctctgcgtgttgacttttgagtaaacttttttttagtagactctgggtaaactctataaattctaagtaaattcggtaaactctcgagtttactaccgagtcaacaagttagcaagttaaaaaaattagatcaaaatgtaagttattttttattgttttctgtttagcattcaacataccttcatttaggatgttattctcaaatacaaatttcttacatttaataatatcaaacttttattctctaataacatcaaatccttgatgagaatgatttaccactactctaacctctacaagttattatctagtagtgatgtattattactatacttggttatttaaatattttgaactttatgatttactattttgctttattatattattgaaatgtttaattagtatgttatttatagatattttattattatttttatatgaagtagactcttacgagtctacgagttgaatctatgaaattctcacaagtatgtgtaaactcttgagtttgataacctttcatgtatctttcatttttatttaaataatttgaaaatttaaaaattattccagagaatattgaaatgtaatctcacacacatatttaaaatactcaaaatactcagaaatgatatttctgtgttatatttctgggataacattcctaggaatgaaaaaaaaatccatgaaataaacgccccttaagatttgtaccctctttctaagttataggcctttcatatcatattagttacattagtttggtttgtttgagaagctcacttaaccaagtaaaatagggcttgcttaagcaagcacaacagaaattgaagccttcataaatgtggctcgcttaagctaggttaatgatttttttagggtccaaacgcaaggttcacttaatcgagtgagcatgacttgcttaggcaagttcagtgactcaagggaccttcaacttccatcattcaacgaacacgctcttatagctctccaaaaatgattagtgaaattccttcaaaacaaacaaaaaaaagtaaagataaaatggaaaaggtgtagaaactaaatttctaacttgaaaacaattttattctaaatttaactaaacaagatacgaacataattcaaaagcacaagacatatataataattccactaacaatataaaagaatatttattattgaaagtgagatagtctccacttacaccttttttctttaaaaaaatatgccatatcatcttacttcttttcaagttttattataaaaatcttaaattttttactattgaacttttatggttatgttaggattttccttttttacatttactttttttttactttaaatttattttttgactcctcaaattttagttgcatataatttcatcaaattaatttatttcaaatattcatttttcctacaaatcttatttacttatttcaaattagtaaataatagtataatatcttaagaataatttagtattttagtctattctttctaatgccatttttaagttcttttcatttaaagtttaaagttttaaattaagtatgcatatcattaaatgtaatttaaaatatttttaaatttaacaaaaaaactaccttctatttaatttggttgtatatgattcaattttgacacaacctcaattttgattactatagagtaaaaatggaggtatacctgcgtgggagccagtaagatctcaaaaatggttggaagtaagtgttacattgatggacaatacaatgcacataaattcactaattgatgaaatatatttgctactaatgtctaacgttaatggattggttatttttgtcatcaatttgtagcgactaaatcccacggaattttatgctgacttaataattgagaatgactatgttgaatgcacaggatcggcaattcaaagtttagtgtttttcaaaagtctatatccagagtataggaagaaagagatagagaatttcattaacaaagcagtacaattccttgaagatgaacaatcaactgatggttcttggtctggtaagtggggagtttactccacttattcttcctggtttgcacttcgaggtctagctgcagctggcaagacttatacaaattgtgttaccattcgcaaagctgtgaaatttctactttcaatacagactgaagatggtggttggggagagagttatctttcaggcgcaaaaaatgtttgtaattaaatgattatagtttccttcaaattcaatgttaagaagtataaaagaagtattattaatattttcttaatgaataaatgtgtataatgtagacgtacgtacctcttgaaggaaatcgatcaaatgtcgtacaaacagcttgggctctaatggctctaattcatgctgggcaggtattgttttaatatgtgcatgtgaaatagattacaggtccacttcaccgtatcttggattaaaacatgattttcttatacgcgtttcaaaataaatgtatttcttatccttttttttaatatatgtaggctgagaaagatccaactcctattcacaatgcagcaaagttactcatcaattctcagcttgaagatggtgattggcctcaacaggtacctctcaaatctcgatccttgataatacagttgttactaatgtgtatgctttgttaatagtagcaacaattaaatatttagcaatcaaataaatataaatacaattaaataattttttttaaaagctaaagattatatttattaaataatagaatggacagttacaaatctaggtcttacaaccaaaactttgtgaataagccacattgtcagaatgtgtgtttgtgaaaaacgaatattataaatctaggtcttagaacaatacatgaatgaacaacattaaaagttggttagtagtcacatgattactttaaaaagttatatgcattcatattttaattttcatcattcaaagatttttttttaacagccaacaaaacaatatattaataataggtaaaggggtaccaaacccatataataaaaaaaaacagataacagtatccatctgcaaagatatacctcccaatgattacactatatttatgagacagaagaggcaccataaatgtagtactatagccatcatgtattccaagactaagtacagtacaaattcagtgaaataataactagctctgttatagaaattgccatccgaaaaggagccaaatggtgctctccaccacctaaaaccaaactgttatgccgtccccgtgagagcaaaaggaaagttattttttccataagacagttgaaggttggcaatccaagtaaaaaaaacagtagttgaagaagttctgtttggcttttaaatttgatttaatgattcaaacttatagttcccatttctcacaaaaactagccagaattctcatttgatgcgcttcctatatatgtgggataggatcaatttacatcagatgtcaaacttttacacttatacgacttaataactttttatagatattataaaatcaatcaattcaaaccattcgatcatatttcatattctctttacaattatgtatatataaagcttcaaacaagttaggcatttgtttatatgttatctaacaatggacatttaattaattatattttaaatagcatattatccataatttatgtagtgtcatgaagtagcaattggtttttgatttataaaaaatttataagcttgatctactttatattatttgtgtgtgtgttgatttattgtaacagtacactaattgtacttagtaattgatttgaaattgatgcattgaaattgagtttctatttttaattcttggttgtaacttatttattgtaggaagtcacaggattattcttgaagacaggcgcattacactacccgctctatagaaattattttccaatgtgggctttgagtgaatatcgtaaaaaagttatgttgccttgtactgctgcttaa

SEQ ID NO: 6 - protein, 762 aa, Glycine max
MWRLKIADGGNDPYIFSTNNFVGRQTWEFDPEAGSPEERAQVEAARQHFYHNRFKVKPCADLLWRFQVLRENNFKQTIPRVTIEDGEEITYQKVTSAVRRGAHHLAALQTSDGHWPAQIAGPLFFLPPLVFCMYITGNLESVFPEEHRKEILRYTYYHQNEDGGWGLHIEGHSTMFCTALNYICMRMLGEGPNGGHDNACARARKWIRDHGGVTHIPSWGKTWLSILGVFDWCGSNPMPPEFWILPSFLPMHPAKMWCYCRLVYMPMSYLYGKRFVGPITPLILQLREELFTQPYEKVNWKKARHQCAKEDLYYPHPLIQDLIWDSLYIFTEPLLTRWPFNKLIREKALQVTMKHIHYEDETSRYITIGCVEKVLCMLACWVEDPNGDAFKKHLARVPDYLWVSEDGMTMQSFGSQEWDAGFAVQALLATNIIEEIGPTFAKGHDFIKKSQVKDNPFGDFKSMHRHISKGSWTFSDQDHGWQVSDCTAEGLKCCLLLSMLPPEIVGEKMEPERLYDSVNVLLSLQSKKGGLAAWEPAGAQEWLELLNPTEFFADIVVEHEYVECTGSAIQALVLFKKLYPGHRKKEIENFITNAVRFLEDTQTADGSWYGNWGVCFTYGSWFALGGLAAAGKTYTNCAAIRKAVKFLLTTQREDGGWGESYLSSPKKIYVPLEGSRSNVVHTAWALMGLIHAGQADRDPMPLHRAAKLLINSQLEEGDWPQQEITGVFMKNCMLHYPMYRDIYPMWALAEYRRRVPLPSTEV

SEQ ID NO: 7 - protein, 762 aa, Glycine max
MWRLKIAEGGNEAYIFSTNNFVGRQTWEFDPEAGTPEERAQVEAARKDFYHHRFKVKPCADLLWRFQILRENNFKQTIPSVKIEDGEEITYQKVTSTVRRGAHHLAALQTSDGHWPAQIAGPLFFLPPLVFCMYITGHLESVFPEEHRKEILRYIYYHQNEDGGWGLHIEGHSTMFCTALNYICMRILGEGPNGGHYNACARARNWIRDHGGVTHIPSWGKTWLSILGVFDWCGSNPMPPEFWILPSFLPMHPAKMWCYCRLVYMPMSYLYGKRFVGPITPLILQLREELFTQPYEKVNWKKARHQYAKEDLYYPHPLVQDLIWDSLYIFTEPLLTRWPFNKLIREKALQVTMKHIHYEDETSRYITIGCVEKVLCMLACWVEDPNGDAFKKHLARIPDYLWVSEDGMTMQSFGSQEWDAGFAVQAFLATNLIEEIGPTLAKGHDFIKKSQVKDNPLGDFKSMYRHISKGSWTFSDQDHGWQVSDCTAESLKCCLLLSMLPPEIVGEKMEPERLYDSVNVLLSLQSKKGGLAAWELAGAQEWLELLNPTEFFADIVVEHEYVECTGSAIQALVLFKNLYPGHRKKEIENFIANAVRFLEDTQTADGSWYGNWGVCFTYGSWFALGGLAAAGKTYINCAAIRKAVKFLLSTQREDGGWGESYLSSPKKIYVPLEGSRSNVVHTAWALMGLIHAGQADRDPKPLHRAAKLLINSQLEEGDWPQQEITGVFMKNCMLHYPMYRDIYPMWALAEYRRRVPLPSTEV

SEQ ID NO: 8 - protein, 762 aa, Glycine max
MWRLKIADGGKDPYIFSTNNFVGRQTWEFDPEAGTPEERAQVEAARQNFYNNRFKVKACGDLLWRFQILREKNFKQSIPSVKIEDGEEITYEKVISTLRRAAHHLSALQTSDGHWPAQIAGPLFFLPPLVFCMYITGHLDLVFPEEYRKEILRYIYYHQNEDGGWGLHIEGHSTMFGTTLNYICMRILGEGPNGGHENACARGKKWIHDHGGVTHIPSWGKTWLSILGVFDWCGSNPMPPEFWIIPSFLPMHPAKMWCYCRLVYMPMSYLYGKRFVGPITPLILQLREELFTQPYEKVNWKKVRHQCAKEDLYYPHSLIQDLVWDSLYMFTEPLLTCWPFNKLIREKALQVTMNHIHYEDENSRYITIGCVEKVLCMLACWVEDPQGDAFKKHLARVSDYLWVSEDGMTMQSFGSQEWDAGFAVQALLATKLIDEIGHSLAKGHDFIKKSQVRDNPSGDFKSMYRHITKGSWTFSDQDHGWQVSDCTAEGLKCCLLLSKLSPEIVGEKVKPERFYDSVNILLSLQSKKGGIAAWEPIGAQEWLELLNPTEFFEDIVIEHEYVECTGSAIQALVLFQKLYPEHRKTEIKNFIVNAVQFLEDTQTTNGSWYGCWGVCYTYGSWFALGGLAAAGKTYTNCNAIRKAVKFLLTTQREDGGWGESYLSSPKKIYIPLEGSRSNVVQTAWALMGLIYAGQSERDLTPLHQAAKLLINSQLEEGDWPQQEITGVFLKNCMMHYPMYRNIFPMWALAEYRRRVPLPSTEG

SEQ ID NO: 9 - protein, 630 aa, Glycine max
MYITGHLDSVFPEEYKKEILRYMYYHQNEDGGWGLHIEGHSNMFCTALNYICMRVLGEGPNGGHDNACARARKWIHDHGGVTHIPSWGKTWLSILGVYDWYGSKPVPPEFWVLPSFLPMHPSKIWCYCRLTYLPMSYLYGKRFVGPITPLILQLREELFTQPYEKVNWKKARNQCAKEDRYHPHSLIQDLVWDSLYIFIEPLLTRWPFNKLVREKALQVTMKHILYEDENSRYITIGCVEKALYMLACWVEDPNGDAFKKHLARVPDYLWVSEDGMAMQSFGSQGWDASLVIQALLATNLMEEIGPTLAKAHDFIKKTQVKDDPLGDFKSMYRHISKGSWTFSDQDQGWQVSDCTAETLNCCLLLSILPSEIVGEKMEPQRLYDSVNILLSLQSRKGGLAAWEPSGTQEWLEILNPTEFLGNIVVEHDYAECTGSAIQALVLFKKLYPRHREKEIKNSIANAVQFLEDTQTANGSWYGSWGVCYIYGTWFALRGLAAAGKTYTNCAAIRKGVKFLLSTQKEDGGWGESYLSCPKQIYVPLEGSRSTVVHTAWALLGLILAGQAERDTTSLHRAAKLLINSQLKEGDWPQQELTGVFNRTCMLHYPMYRNIYPMWALAEYRRLVPLPSTEV

SEQ ID NO: 10 - protein, 696 aa, Glycine max
FLREKNFRQTITSVKIQDDEEITNEKTTNTLKRAIHYLSALQTSNGYWPAQFSGPLFPTALFVMCMYITGHLDSIFPEEYRKEILRYIYFHQNEDGGWGLNIDGHSTMFSTTLNYICMRILGEGPNGGHNNACAKARKWIHDHGSVTHIPSWGKFWLSVLGVVDWCGSNPLPPEFWILPTFSPMHPAKMWCYCRLIYMPMSYLYGKKFVGSITPLVLNLREELFTQPYDENSWKKARHKCAKEDLYYPHPWIQDLIWDSLYLFVEPLLTRWPLNKLIREKALQVTMKHIHYENENSRYLDMACVEKSLCMLACWVEDPNGDAFKKHLARIPDYFWVSEDGMIVQPVIGSQAWDISFIVQALLATDLIEEFGPTLAKAHDFIKKSQLTDNLPGDFKSMYRHISKGAWTFSHKDDGWQLSDSTAECLKCCLLLSMMPQEIVGEKMEPEKLYDSVDFILSLQSKNGGIPAWEPVRSQKWLERLNPTEFYADLIIENDYVECTGSAIQSLVFFKSLYPEYRKKEIENFINKAVQFLEDEQSTDGSWSGKWGVYSTYSSWFALRGLAAAGKTYTNCVTIRKAVKFLLSIQTEDGGWGESYLSGAKNTYVPLEGNRSNVVQTAWALMALIHAGQAEKDPTPIHNAAKLLINSQLEDGDWPQQEVTGLFLKTGALHYPLYRNYFPMWALSEYRKKVMLPCTAA

SEQ ID NO: 11 - DNA, 192 bp, Glycine max
  source 1..192: mol_type = other DNA
gaagatctttactatccccatcctttgatacaagacctaatatgggatagtttatacatattcactgaaccgctacttactcgttggcctttcaacaagttgattagagaaaaggcccttcaagtaactatgaaacatattcattatgaagatgagactagtcgatacataaccattggttgtgtggaaaag

SEQ ID NO: 12 - RNA, 24 nt, synthetic construct
  source 1..24: mol_type = other RNA
tacatattcactgaaccgctactt

SEQ ID NO: 13 - DNA, 23 bp, synthetic construct
  source 1..23: mol_type = other DNA
gatagtcgttcattatgtcaatc

SEQ ID NO: 14 - DNA, 19 bp, synthetic construct
  source 1..19: mol_type = other DNA
cacacaaccaatggttatg

SEQ ID NO: 15 - DNA, 29 bp, synthetic construct
  source 1..29: mol_type = other DNA
gactatagaagatggagaggaaatcacat

SEQ ID NO: 16 - DNA, 23 bp, synthetic construct
  source 1..23: mol_type = other DNA
aagagaggacctgcaatttgagc

SEQ ID NO: 17 - DNA, 13 bp, synthetic construct
  source 1..13: mol_type = other DNA
ccgtcagatgggg

SEQ ID NO: 18 - DNA, 13 bp, synthetic construct
  source 1..13: mol_type = other DNA
gtcagaaggggcg

SEQ ID NO: 19 - DNA, 24 bp, synthetic construct
  source 1..24: mol_type = other DNA
tagagcaagaaagtggattcgaga

SEQ ID NO: 20 - DNA, 26 bp, synthetic construct
  source 1..26: mol_type = other DNA
caccgagtatctacaagagcaagatc

SEQ ID NO: 21 - DNA, 13 bp, synthetic construct
  source 1..13: mol_type = other DNA
tcatgggaaaaaa

SEQ ID NO: 22 - DNA, 15 bp, synthetic construct
  source 1..15: mol_type = other DNA
cttcatggggaaaaa

SEQ ID NO: 23 - DNA, 28 bp, Glycine max
  source 1..28: mol_type = other DNA
tttatacatattcactgaaccgctactt

SEQ ID NO: 24 - DNA, 28 bp, Glycine max
  source 1..28: mol_type = other DNA
tctatacatgtttactgagccactactt

SEQ ID NO: 25 - DNA, 28 bp, Glycine max
  source 1..28: mol_type = other DNA
tttatacatatttattgagccactactt

SEQ ID NO: 26 - DNA, 28 bp, Glycine max
  source 1..28: mol_type = other DNA
tttatacatatttactgagccattgctt

SEQ ID NO: 27 - DNA, 28 bp, Glycine max
  source 1..28: mol_type = other DNA
tttgtatttgttcgttgagccactcctt

SEQ ID NO: 28 - DNA, 66 bp, Glycine max
  source 1..66: mol_type = other DNA
cctaatatgggatagtttatacatattcactgaaccgctacttactcgttggcctttcaacaagtt

SEQ ID NO: 29 - DNA, 56 bp, synthetic construct
  source 1..56: mol_type = other DNA
cctaatatgggatagtttatacatattcactgaactcgttggcctttcaacaagtt

SEQ ID NO: 30 - DNA, 38 bp, synthetic construct
  source 1..38: mol_type = other DNA
cctaatatgggatagtctcgttggcctttcaacaagtt

SEQ ID NO: 31 - DNA, 62 bp, synthetic construct
  source 1..62: mol_type = other DNA
cctaatatgggatagtttatacatattcactggctacttactcgttggcctttcaacaagtt

SEQ ID NO: 32 - DNA, 56 bp, synthetic construct
  source 1..56: mol_type = other DNA
cctaatatgggatagtttatacatattcactgcactcgttggcctttcaacaagtt

SEQ ID NO: 33 - DNA, 23 bp, synthetic construct
  source 1..23: mol_type = other DNA
actcgttggcctttcaacaagtt

SEQ ID NO: 34 - DNA, 61 bp, synthetic construct
  source 1..61: mol_type = other DNA
cctaatatgggatagtttatacatattcactgctacttactcgttggcctttcaacaagtt

SEQ ID NO: 35 - DNA, 58 bp, synthetic construct
  source 1..58: mol_type = other DNA
cctaatatgggatagtttatacatattcactgattactcgttggcctttcaacaagtt

SEQ ID NO: 36 - DNA, 58 bp, synthetic construct
  source 1..58: mol_type = other DNA
cctaatatgggatagtttatacatattcactgcttactcgttggcctttcaacaagtt

SEQ ID NO: 37 - DNA, 61 bp, synthetic construct
  source 1..61: mol_type = other DNA
cctaatatgggatagtttatacatattcactgaaccttactcgttggcctttcaacaagtt

SEQ ID NO: 38 - DNA, 8570 bp, Glycine max
  source 1..8570: mol_type = genomic DNA
gagaaagatcaacttgataaaaaaaaaaaagaagaagttatgattaaaatgaattgtaaattattattatattgaacagttaaaatagaatgagaatgttattaaatgttttagcggtataaataatgggagtagctggagcttagagaaagaggtttgtttggtgtgagtgaatagggatcagggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggtaccctacgttacctatctatttcccaacttaatttaattactctctcttctattcttatttgccctacgcgtaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcctttggtaagtactcactactcactacattagtttatcatcgtgtttctcttttattaattccatctcataaataacaaaactaaatccatttgattccagttttgcttaggagtaaaactaattcaaatgaatcaatcatattactgaatcatgttagtgtacaaacacgcgcgcattcaccttacctacttttatgtttctcttgtaagctactttatgttacttctgccttaattaataacatcctaaacatttttttggattatttgtctctttaattctattatagtcattttttcatttcgtatagtttatattttagtttttttttattcttagtttatattttaattttttttaattttgaaagtgatatttttagttcctataatttatattttaattatcttttaattcatggtaaataaaatataaaaataattaattataaattcttaactattttttattacaaattatcttgcaataaattaattataaattatttgttaatatttttataattaattataataaaaaatattaattatattttgatgataatgactaaaataaaattaaaatataaaatacaagattaaaaaaaactactttaaaattatatggattaaaatagaattaaaatgtaaattataaagaataaaaatatcattttcttttacctaaatgaaaaaaaaaattaaaatataaattataaggattaaaatattattttcaaataactaaaaagtataaattataaaattataaagaataaataaataattattttctttttgtttcaagatatcctaaacatacgcttcattgttatatatatatataatccaaataaaaccaatatgaccagtttaagctagtgggtaggcgcatattaaaataaaaaacacgaattattattattattattattattattattattattattattattattattattattattatatgttttaaagaactctgtcctcaatcattgtctaatataagttatttgttcatcaacaatcgagatagtaatcaaatcagttagaaatgttgatattgcattttatatcaatgtttatatgcttttattgtttaatctttacatcttcaaatttatttaagatatgaggacaaaataaataaaatgaaaggcatgttgggataagaagaaatttttaattagcaaaaaaatagttatttctaaaataattttttattttattcgattaaatttttttacaacaaaatttagatataaatatctagatttttttgtttgattctttaatttaaatgaaaattttatctcactaatttatgtcaatatttaatgtaaacttttatcatgtaaatgattgatgtaaaattctaattttgattacaataatataaaaaaaagactgtttcgtaggtttgacgtttttaatacattttaaaattttacgtcaaatcaatattagatgcgcaaaattcagattggacgtggatctacacgcacgtagcgttaatgacgagggtgttggcataaataagaaatatacaaaaatgaaagacgtcactatcattatctaaaatggatagtttacaggcttgttgatcataataatttattggattaattagtttttttttagtatttataaaattttaaatttcaagtattagtctctataaaaaaaattatttagtctcattaattttattgctcgtattaatttctgagctaaaattttctcttattgcctattcttagatccttctctctggactaattataaacaataaaaataaacaatacacccaaaaattagaggcagttctttatgtcaattctatacaataaatcctcagtaagctcatagtatactcttttaaaaaaatacaagtaatcactttcgtattctctgaccaaatttactatcaatttaatttttaaataataataattattagagagtatagataaattagctgatgatgcatgtctgggatatatcaattttgtgaatattaaaaataaaataaaattatacattgataaaataagataataattttacatcatcataacaaatcattattatgataagtttattaatttttataataactatcttaataatcatactaataatgaattataactatcttaataattcatatgaacaatgaattataattattagatgtaatgtaaaattattttagattgtcagtacattgattattaaatttttatttttattttttttaaagaaattagaaaattgaaattaaattaaatatgcagcttaggtaataaaaaaattaatatccttggtctcttttttacggaactaaacttataaaagaatgaaatagggatttattcattaacagaataattcaggtcatcattttatgccatactcgaccattaaatgaacataaaaacttagttattgtacatgaaattgaaaatgatcactataattaagaatccaaaattattttcattttttgtttgctcacaagtactcctcttttttacataatcatgaaatatctcagatattttgaacatcatatatataaaattcaagttcattatgcatatgcaataacaaacattaatttttacttgttgaaaatattgcaggttttttgtatgtatattacaggaaatcttgaatcagtatttccagaagaacatcgcaaagaaattcttcgttacacatattatcaccaggtataattattcaatcttaagatacatttgcttaatcaaacataatatagagaaacaaaatgtcaaatttcttgtatcttttaaacttgttgtacttgtttaaccatgaagtttataatctattcattgcaacaataaataattaatattagcccttttgtgctttcatttaattaaagaatgaagacggaggatggggactacacatagagggtcatagcactatgttttgtactgcactgaactatatatgcatgcgaatgcttggagaaggacctaatggaggtcatgacaatgcttgtgctagagcaagaaagtggattcgagatcatggtggtgtaacacatataccttcatggggaaaaacttggctttcggtattattcttttttatatgaatacaaatacaaaaactaatgaaactatgatgtgtactattcatttaaatggatcttgctcttgtagatactcggtgtatttgattggtgcggaagcaacccaatgcccccagagttttggatccttccatcttttcttcctatgcatccaggtagatttaattaattttattttgtcactctgtttggaatgatatatatttaaattggtgaatatacaatatttaatcattaaatctcacttccaccttaactattgtagctaagatgtggtgttactgtcgattggtatacatgcctatgtcttacttatatgggaagaggtttgtgggtccaatcacaccactcatcttacaattaagagaagagttgtttactcaaccttatgaaaaagttaattggaagaaagcgcgtcaccaatgtgcaaaggtgaaagacatatttaattaatcacaatgtgtgtgtgcacatatcttttttgctatgaaaatgatagtcgttcattatgtcaatcacaggaagatctttactatccccatcctttgatacaagacctaatatgggatagtttatacatattcactgaaccgctacttactcgttggcctttcaacaagttgattagagaaaaggcccttcaagtaactatgaaacatattcattatgaagatgagactagtcgatacataaccattggttgtgtggaaaaggtaggtgtcataacaataatgcatattgattttttttatccttatgagtaaaataaactttttattttccttttcaagagggaattcattcaaatatgatactaaaatgcatatattttttttccaggttttatgtatgcttgcttgttgggtggaagatccaaacggagatgctttcaagaagcatcttgcaagggtcccagattacttatgggtttctgaagatggaatgaccatgcaggtatacgcgtgtgggcacgtgctattataaccaattagatacattaattatttaattagtttattgatgcacttatctatataatgcagagttttggtagccaagaatgggatgctggctttgctgttcaagctttgcttgccactaacataattgaagaaattggtcctacgtttgcaaaaggacatgatttcatcaagaagtctcaggtttgcttaatatttttataagtatataaaagaatcccagatcttcttccttctagttatggagcagaaattccctaataaacctaggtttatccagattgtacaattaactaattaagatatttaaaatccctaaatcttatttacagggtgatcgaataaactaaataaaaactatataactaattacttactaattcacctagctacatagtattagttgtaactagtataattatatttttgaattgaaatgcatatttataattgattactaagttggtgtttaactatttattagagtattgaataatatggttgttgtctatatctagtagtaatgaactagcattttcattttgtaggcgaaggataacccttttggagatttttatgaattggcattcttattttataggtgaaggataatccttttggagattttaaaagtatgcatcgtcatatttctaaagggtcttggacattctctgatcaagaccatggatggcaagtttctgattgcactgcagaaggtttaaaggtagccaacaacttaggtcataaacaacttaatctaaagctttgttaatgatcacaacaaatatatgttaatttaactaaaatttttgtgttgtagtgttgtctacttctatcaatgttgccaccagagattgtgggagaaaagatggaacctgaaagattatacgattcagtcaatgtcttgttgtcgcttcaggcaagtaccattaagtttagagaaacgatttatgttatgaaatattttataaaaaattgtctataaattgataaaggttggcaactaatgttgatataatcagctgttttttcaaatccaacaccacctcatttgttcatgatttgtgaaattggttttgtacaatattacaattctcttaaacttatgtccatatccttttaattaaccaatcatcaatcaactaatggccccttttctttgttacttaatatttgatcaataataagttagggttgtacaaaggactttgatcattgatgctttatttacttttactaccaaatttatttttaactaaacaacattttttttattgcagagtaaaaaaggtggtttagcagcatgggagcctgcaggagctcaagagtggttagaagtaagtattaaattaattaataatgcaacacgtatgtatttataaattgataaaaaaaagtacttgattaataaactaattaactttgttggtgattcttagttactcaatcccacagaattttttgcggacattgtagttgaacatgaatatgttgagtgcactggatctgcaatccaagctttagttttgttcaagaagctatatccaggacataggaagaaagagatagaaaatttcattaccaatgcagttcgattccttgaagatacacaaacagctgatggttcatggtatggaaattggggagtttgcttcacttatggctcttggtttgcacttggaggtctagcagctgctggtaagacttacaccaattgtgctgccattcgcaaagccgttaaatttctacttacaacacaaagagaggacggtggatggggagagagttatctttcaagcccaaaaaaggtttgtagctgaatggttattactgaattaccattcttcaacttcattgggaattaatgataatagtaataatgtcttactaaatgaaaataatgcagatatatgtacctctagaaggaagccgatcaaatgttgtacatacagcatgggctcttatgggactaattcatgctggacaggttcgttcttaatttgtgtttgtgtatatttgattataggtctacttcattgtgaattaaatttcaaaatagtttttttttcccctcttcatttttaaattcctatatacaggcggatagagaccccatgcctcttcaccgtgctgcaaagttgctcattaattctcagttggaagagggtgattggccccaacaggtgtatatcaatcctttaaattaaaaaatgtcccgtatttgtgtacatgcttaactaaataaataaataaaaaagaaattaacttatttagcaataaattaaaatcagatcttagaaaaaaagacataggtcgataccatttgggacaagacttcagtgcaacaattcagatgccttgggtgttttaaaatcaaaattaaagtttgacctagctaaagaaattgaaagttgatagatgctattgatgtgaaattctaattttaattggaaaataattccaaaacgtaaaatacaacatttaagttttgtggacaaagttatttctttaaaattttgcttgaactagtattttagtttgtttttataataggattaatttttattttatataattataattcataataaataaatatgttataattaaaatttataatttataataaataaagatatttaaatataaaaattataatttgaatttataataatcaatttaaattgtatggtaggtgttaagtgtattttatattttcaagacaataaatgcatttattattgtatttaataattcagtcgttccataataaatattagaagaaaaataaattctaaaataagtgttatttatactattagtgcatacactttttctctaactgctaatgcttgcaattcatatgcaatactctttgttttattttgattaacctttagagaaaaaaattgacacaatattttagaaaatacaattaataatttagatttcataaaaattgttatgtaatttcctaatcctaatatgtcaatgtatttcatattaattagattatctctcatctatacttttttattttctcactctacatttattgattaatgaagaaaaaaaaagtaattcttcattggttttgtaaaatgacaatcaaaacaatttttttctaaaacatcaattaaaatgaaatggaaggagtaaaatcaaagtctttaaaactttgtcgatacgagaatttcaacttccaatgtaatcaaaacttgatataaaaatattaatatttttttaatttaggaaaatgacagaaaagatttattattaattgtattttgtaagcaaaatgaaaattatgctctagattgagtattgtttcttattgttgtgacctacttttggcaggaaatcacgggagtattcatgaaaaattgcatgttgcattatccaatgtacagagatatttatccaatgtgggctctagctgaatatcgaaggcgggttccattgccttccactgaagtttaatttagaatggtttgagcacgaaaaggcaaaggcattttcattaagattgaggcaaataagttgtgtgtaatcaagcttaatcaattttttcatattcctatgtttatttcctacatatattggtagaaaaattatttcatgtcggcttgtatagatgttcttaatgttcgtgttacgatacaaagataagtctagtaaaatactacgcacgtaacattagagggctgtgaagaagataaaaatgttaccaagaaaattaattagtttctcagtgatactcaatacacagggtcgtgcctatggtaaatagggatccacaagaaata

(19) United States
(12) Patent Application Publication
(10) Pub. No.: US 2023/0340515 A1
(43) Pub. Date: Oct. 26, 2023

(54) COMPOSITIONS AND METHODS COMPRISING PLANTS WITH MODIFIED SAPONIN CONTENT

(71) Applicant: Benson Hill, Inc., St. Louis, MO (US)

(72) Inventors: Matthew Brett Begemann, St. Louis, MO (US); Emma Elizabeth January, St. Louis, MO (US); Erin Zess, St. Louis, MO (US); Herbert Wolfgang Goettel, St. Louis, MO (US)

(21) Appl. No.: 18/194,241

(22) Filed: Mar. 31, 2023

Related U.S. Application Data
(60) Provisional application No. 63/326,614, filed on Apr. 1, 2022.

(51) Int. Cl. C12N 15/82; A01H 6/54; C12Q 1/6895

(52) U.S. Cl. CPC C12N 15/8245; A01H 6/542; C12Q 1/6895

(57) ABSTRACT

Provided herein are plants and plant parts comprising reduced saponin content. The plants and plant parts can have a genetic mutation that decreases the beta-amyrin synthase (BAS) activity, one or more mutations in at least one BAS gene or homolog or in its regulatory region, decreased level or activity of the BAS gene or BAS protein, and/or improved flavor characteristics. Also provided herein are compositions and methods of producing such plants and plant parts, and plant products including plant protein compositions comprising reduced saponin content and/or improved flavor characteristics.

### FIELD OF THE INVENTION

The present disclosure relates to plants and plant parts having decreased saponin content, comprising decreased beta-amyrin synthase activity, and associated methods and compositions thereof.

### RELATED APPLICATIONS

This application claims priority to U.S. Provisional Application No. 63/326,614 filed on Apr. 1, 2022, the content of which is incorporated herein by reference in its entirety.

### SEQUENCE LISTING

This application contains a Sequence Listing which is submitted herewith in electronically readable format. The Sequence Listing file was created on Mar. 29, 2023, is named “B88552_1400_SL.xml” and its size is 103 kb. The entire contents of the Sequence Listing file are incorporated by reference herein.

### BACKGROUND OF THE INVENTION

Bitterness compounds in plants, plant parts, plant compositions, or plant-based food and beverage products produce undesirable, off-putting flavors. Removing such flavors or masking them (for example with salt and sugar) in the processing of plants adds processing costs, energy, and labor and/or makes final product formulations less healthy. Therefore, reducing the amount of bitterness or off-flavor compounds in the crop may add value.

Saponins are among such bitterness compounds found in plants. Saponins (e.g., group A saponins, group B saponins, 2,3-dihydro-2,5-dihydroxy-6-methyl-4H-pyran-4-one (DDMP)-saponins, group E saponins) are amphiphilic glycosides of steroids and triterpenes and are known to cause “bitter”, “beany”, and “astringent” flavors, limiting inclusions of saponin-containing plant compositions in various food applications. Saponins are synthesized via a terpenoid pathway in plants. Accordingly, decreasing saponin content in plants or plant parts, for example by inhibiting the saponin synthesis pathway, could have important commercial advantages, particularly in view of the growing plant-based meal market, in which soybean meal offers the leading source of protein, e.g., as diary and meat substitute. Decreasing saponin content in plants or plant parts could also offer commercial advantages in aquaculture feed market, in which inclusion of some plant (e.g., soybean) based meal is currently limited due to saponins therein, which cause various pathologies in fish species such as Atlantic salmon.

### SUMMARY OF THE INVENTION

Plants and plant parts comprising a genetic mutation that decreases the beta-amyrin synthase (BAS) activity are provided. Compositions and methods for producing such plants and plant parts, and products (e.g., protein compositions) produced from such plants and plant parts are also provided. The plants or plant parts of the present disclosure can have one or more mutations in at least one native BAS gene or homolog or in its regulatory region, decreased expression levels of the BAS gene, decreased levels or activity of the BAS protein, decreased saponin content, and/or improved flavor characteristics compared to a control plant or plant part.

In one aspect, the present disclosure provides a plant or plant part comprising decreased beta-amyrin synthase (BAS) activity compared to a control plant or plant part, wherein said plant or plant part comprises a genetic mutation that decreases the beta-amyrin synthase activity. In some embodiments, the plant or plant part comprises decreased saponin content compared to a control plant or plant part. In some embodiments, the plant or plant part comprises improved flavor characteristics compared to a control plant or plant part.

In some embodiments, the mutation comprises one or more insertions, substitutions, or deletions in at least one native BAS gene or homolog thereof or in a regulatory region of said at least one native BAS gene or homolog thereof in said plant or plant part, wherein an expression level of said at least one mutated BAS gene or homolog thereof is reduced compared to an expression level corresponding at least one native BAS gene or homolog thereof without said mutation. In some embodiments, the mutation comprises one or more insertions, substitutions, or deletions in at least one native BAS gene or homolog thereof or in a regulatory region of said at least one native BAS gene or homolog thereof in said plant or plant part, wherein said mutation reduces level or activity of the BAS protein encoded by said at least one BAS gene or homolog thereof compared to the level or activity of a BAS protein encoded by corresponding at least one native BAS gene or homolog thereof without said mutation.

In some embodiments, the mutation is located in a BAS gene or homolog thereof: (i) comprising a nucleic acid sequence having at least 80% sequence identity to a nucleic acid sequence of any one of SEQ ID NOs: 1-5 and 38, wherein said nucleic acid sequence encodes a polypeptide that retains BAS activity; (ii) comprising the nucleic acid sequence of any one of SEQ ID NOs: 1-5 and 38; (iii) encoding a polypeptide comprising an amino acid sequence having at least 80% sequence identity to an amino acid sequence of any one of SEQ ID NOs: 6-10, wherein said polypeptide retains BAS activity; (iv) encoding a polypeptide comprising an amino acid sequence of any one of SEQ ID NOs: 6-10; and/or in a regulatory region of said BAS gene or homolog thereof.

In some embodiments, the BAS gene or homolog thereof in the plant or plant part comprises a BAS1 gene. In some embodiments, the mutation is located in a BAS1 gene or homolog thereof: (i) comprising a nucleic acid sequence having at least 80% sequence identity to a nucleic acid sequence of SEQ ID NO: 1 or 38, wherein said nucleic acid sequence encodes a polypeptide that retains BAS activity; (ii) comprising the nucleic acid sequence of SEQ ID NO: 1 or 38; (iii) encoding a polypeptide comprising an amino acid sequence having at least 80% sequence identity to an amino acid sequence of SEQ ID NO: 6, wherein said polypeptide retains BAS activity; (iv) encoding a polypeptide comprising an amino acid sequence of any one of SEQ ID NO: 6; and/or in a regulatory region of said BAS1 gene or homolog thereof.

In some embodiments, at least one of said one or more insertions, substitutions, or deletions is at least partially in a nucleic acid region of exon 2, 4, and/or 7 of the Glycine max BAS1 gene. In some embodiments, the plant or plant part comprises a deletion of about 4-78 nucleotides at least partially in the nucleic acid region of exon 7 of the Glycine max BAS1 gene, a substitution in the nucleic acid region of exon 4 of the Glycine max BAS1 gene, and/or a substitution in the nucleic acid region of exon 2 of the Glycine max BAS1 gene. In some embodiments, the plant or plant part comprises: (i) a mutated Glycine max BAS1 gene comprising a deletion of nucleotides 4191 through 4195 of SEQ ID NO: 1; (ii) a mutated Glycine max BAS1 gene comprising a G to A substitution of nucleotide 3564 of SEQ ID NO: 1 or a G to A substitution of nucleotide 3750 of SEQ ID NO: 38; (iii) a mutated Glycine max BAS1 gene comprising an A to T substitution of nucleotide 374 of SEQ ID NO: 1 or an A to T substitution of nucleotide 560 of SEQ ID NO: 38; (iv) a mutated Glycine max BAS1 protein comprising a G to E substitution of amino acid 220 of SEQ ID NO: 6; (v) a mutated Glycine max BAS1 protein comprising an R to W substitution of amino acid 100 of SEQ ID NO: 6; (vi) a mutated Glycine max BAS1 gene comprising a deletion of nucleotides 4190 through 4199; (vii) a mutated Glycine max BAS1 gene comprising a deletion of nucleotides 4171 through 4198 of SEQ ID NO: 1; (viii) a mutated Glycine max BAS1 gene comprising a deletion of nucleotides 4187 through 4190 of SEQ ID NO: 1; (ix) a mutated Glycine max BAS1 gene comprising a deletion of nucleotides 4189 through 4198 of SEQ ID NO: 1; (x) a mutated Glycine max BAS1 gene comprising a deletion of nucleotides 4120 through 4197 of SEQ ID NO: 1; (xi) a mutated Glycine max BAS1 gene comprising a deletion of nucleotides 4187 through 4191 of SEQ ID NO: 1; (xii) a mutated Glycine max BAS1 gene comprising a deletion of nucleotides 4188 through 4195 of SEQ ID NO: 1; and/or (xiii) a mutated Glycine max BAS1 gene comprising a deletion of nucleotides 4187 through 4194 of SEQ ID NO: 1.

In some embodiments, said mutation comprises an out-of-frame mutation of the at least one BAS gene or homolog thereof. In some embodiments, said mutation comprises an in-frame mutation (e.g., a missense mutation) of the at least one BAS gene or homolog thereof.

In some embodiments, said plant or plant part comprises 2-5 genes encoding a BAS protein. In some embodiments, said 2-5 genes have less than 100% sequence identity to one another.

In some embodiments, said plant or plant part is a legume. In some embodiments, said plant or plant part is selected from soybean (Glycine max), beans (Phaseolus spp.), common bean (Phaseolus vulgaris), fava bean (Vicia faba), mung bean (Vigna radiata), pea (Pisum sativum), chickpea (Cicer arietinum), peanut (Arachis hypogaea), lentils (Lens culinaris, Lens esculenta), lupins (Lupinus spp.), white lupin (Lupinus albus), mesquite (Prosopis spp.), carob (Ceratonia siliqua), tamarind (Tamarindus indica), alfalfa (Medicago sativa), barrel medic (Medicago truncatula), birdsfood trefoil (Lotus japonicus), licorice (Glycyrrhiza glabra), and clover (Trifolium spp.). For example, a plant or plant part of the present disclosure can be Glycine max or a part of Glycine max.

In some embodiments, said plant or plant part is corn (Zea mays), Brassica species, Brassica napus, Brassica rapa, Brassica juncea, rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

In some embodiments, said plant or plant part is a seed.

In one aspect, the present disclosure provides a population of plants or plant parts comprising the plant or plant part provided herein, wherein the population comprises decreased beta-amyrin synthase (BAS) activity, a decreased saponin content, and/or improved flavor characteristics compared to a control population. In some embodiments, said plant or plant part is a seed, and said population is a population of seeds.

In one aspect, the present disclosure provides a method for decreasing saponin content in a plant or plant part, said method comprising introducing a genetic mutation that decreases beta-amyrin synthase (BAS) activity into said plant or plant part, wherein BAS activity is decreased and saponin content is decreased in said plant or plant part relative to a control plant or plant part. In some embodiments, the method further comprises introducing the genetic mutation that decreases BAS activity into a plant cell, and regenerating said plant or plant part from said plant cell. In some embodiments, the mutation comprises one or more insertions, substitutions, or deletions in at least one native BAS gene or homolog thereof or in a regulatory region of said at least one native BAS gene or homolog thereof in a genome of said plant or plant part, wherein an expression level of said at least one BAS gene or homolog thereof is reduced by said mutation; and/or level or activity of a beta-amyrin synthase protein encoded by said at least one BAS gene or homolog thereof is reduced by said mutation.

In some embodiments according to the methods provided herein, the mutation is introduced into a BAS gene or homolog thereof: (i) comprising a nucleic acid sequence having at least 80% sequence identity to a nucleic acid sequence of any one of SEQ ID NOs: 1-5 and 38, wherein said nucleic acid sequence encodes a polypeptide that retains BAS activity; (ii) comprising the nucleic acid sequence of any one of SEQ ID NOs: 1-5 and 38; (iii) encoding a polypeptide comprising an amino acid sequence having at least 80% sequence identity to an amino acid sequence of any one of SEQ ID NOs: 6-10, wherein said polypeptide retains BAS activity; and/or (iv) encoding a polypeptide comprising an amino acid sequence of any one of SEQ ID NOs: 6-10, or in a regulatory region of said BAS gene or homolog thereof.

In some embodiments, the mutation is introduced into a BAS1 gene or homolog thereof or regulatory region thereof. In some embodiments, the mutation is introduced into a BAS1 gene or homolog thereof: (i) comprising a nucleic acid sequence having at least 80% sequence identity to a nucleic acid sequence of SEQ ID NO: 1 or 38, wherein said nucleic acid sequence encodes a polypeptide that retains BAS activity; (ii) comprising the nucleic acid sequence of SEQ ID NOs: 1 or 38; (iii) encoding a polypeptide comprising an amino acid sequence having at least 80% sequence identity to an amino acid sequence of SEQ ID NO: 6, wherein said polypeptide retains BAS activity; and/or (iv) encoding a polypeptide comprising an amino acid sequence of any one of SEQ ID NO: 6, or in a regulatory region of said BAS1 gene or homolog thereof.

In some embodiments of the methods provided herein, introducing comprises introducing one or more insertions, substitutions, or deletions that is at least partially in a nucleic acid region of exon 2, 4, and/or 7 of a Glycine max BAS1 gene. In some embodiments, said mutation comprise a deletion of about 4-78 nucleotides that is at least partially in the nucleic acid region of exon 7 of the Glycine max BAS1 gene, a substitution in the nucleic acid region of exon 4 of the Glycine max BAS1 gene, and/or a substitution in the nucleic acid region of exon 2 of the Glycine max BAS1 gene. In some embodiments, said mutation comprises a deletion of nucleotides 4191 through 4195 of SEQ ID NO: 1, a G to A substitution of nucleotide 3564 of SEQ ID NO: 1, a G to A substitution of nucleotide 3750 of SEQ ID NO: 38, an A to T substitution of nucleotide 374 of SEQ ID NO: 1, and/or an A to T substitution of nucleotide 560 of SEQ ID NO: 38. In some embodiments, said mutation produces a G to E substitution of amino acid 220 of SEQ ID NO: 6 and/or an R to W substitution of amino acid 100 of SEQ ID NO: 6. In some embodiments, said mutation comprises a deletion of nucleotides 4190 through 4199 of SEQ ID NO: 1, a deletion of nucleotides 4171 through 4198 of SEQ ID NO: 1, a deletion of nucleotides 4187 through 4190 of SEQ ID NO: 1, a deletion of nucleotides 4189 through 4198 of SEQ ID NO: 1, a deletion of nucleotides 4120 through 4197 of SEQ ID NO: 1, a deletion of nucleotides 4187 through 4191 of SEQ ID NO: 1, a deletion of nucleotides 4188 through 4195 of SEQ ID NO: 1, and/or a deletion of nucleotides 4187 through 4194 of SEQ ID NO: 1.

In some embodiments, introducing the mutation comprises introducing an out-of-frame, in-frame, nonsense, or missense mutation into said at least one native BAS gene or homolog thereof.

In some embodiments, the method further comprises introducing editing reagents or a nucleic acid construct encoding said editing reagents into said plant, plant part, or plant cell. In some embodiments, said editing reagents comprise at least one nuclease, wherein the nuclease cleaves a target site in at least one BAS gene or homolog thereof, or a regulatory region of said at least one BAS gene or homolog thereof, in the genome of said plant, plant part, or plant cell, and said mutation is introduced at said cleaved target site. In some embodiments, the at least one nuclease comprises a CRISPR nuclease. In some embodiments, the CRISPR nuclease is a Type II CRISPR system nuclease, a Type V CRISPR system nuclease, a Cas9 nuclease, a Cas12a (Cpf1) nuclease, or a Cms1 nuclease. In some embodiments, the CRISPR nuclease is a Cas12a nuclease or an ortholog thereof.

In some embodiments, the editing reagents comprise one or more guide RNAs (gRNAs). In some embodiments, the one or more gRNAs comprise a nucleic acid sequence complementary to a region of a genomic DNA sequence comprising said at least one native BAS gene or regulatory region thereof in said plant or plant part. In some embodiments, at least one of the one or more gRNAs binds a nucleic acid region corresponding to exon 7 of the at least one BAS gene. In some embodiments, at least one of the one or more gRNAs comprises a nucleic acid sequence encoded by: (a) a nucleic acid sequence that shares at least 80% sequence identity with the nucleic acid sequence of SEQ ID NO: 12; or (b) a nucleic acid sequence of SEQ ID NO: 12.

In some embodiments, the method further comprises contacting the plant or plant part with a mutagen, thereby introducing said mutation into said plant or plant part. In some embodiments, the mutagen is ethyl methanesulfonate (EMS) and/or N-ethyl-N-nitrosourea (ENU).

In some embodiments of the methods provided herein, said plant or plant part is a legume. In some embodiments, said plant or plant part is selected from soybean (Glycine max), beans (Phaseolus spp.), common bean (Phaseolus vulgaris), fava bean (Vicia faba), mung bean (Vigna radiata), pea (Pisum sativum), chickpea (Cicer arietinum), peanut (Arachis hypogaea), lentils (Lens culinaris, Lens esculenta), lupins (Lupinus spp.), white lupin (Lupinus albus), mesquite (Prosopis spp.), carob (Ceratonia siliqua), tamarind (Tamarindus indica), alfalfa (Medicago sativa), barrel medic (Medicago truncatula), birdsfood trefoil (Lotus japonicus), licorice (Glycyrrhiza glabra), and clover (Trifolium spp.).

In some embodiments of the methods provided herein, said plant or plant part is corn (Zea mays), Brassica species, Brassica napus, Brassica rapa, Brassica juncea, rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

In one aspect, the present disclosure provides a plant or plant part produced by the method provided herein, wherein said plant or plant part comprises reduced beta-amyrin synthase (BAS) activity compared to a control plant or plant part. In some embodiments, the plant or plant part comprises decreased saponin content and/or improved flavor characteristics compared to a control plant or plant part. In some embodiments, said plant or plant part is a seed.

In some embodiments, the present disclosure provides a population of plants or plant parts produced by the methods provided herein, wherein the population comprises decreased beta-amyrin synthase (BAS) activity, decreased saponin content, and/or improved flavor characteristics compared to a control population. In some embodiments, said population is a population of seeds.

In one aspect, the present disclosure provides a seed composition produced from the plant or plant part, or a population of plants or plant parts provided herein.

In one aspect, the present disclosure provides a protein and/or oil composition produced from the plant or plant part, the population of plants or plant parts, or the seed composition provided herein.

In one aspect, the present disclosure provides a food or beverage product comprising the plant or plant part, the population of plants or plant parts, the seed composition, and/or the protein and/or oil composition provided herein.

In some embodiments, the seed composition, the protein and/or oil composition, or the food or beverage product provided herein comprises a decreased level of saponin and/or improved flavor characteristics compared to a control composition or product (e.g., produced from a control plant, plant part, or population without mutation).

In one aspect, the present disclosure provides a nucleic acid molecule comprising a nucleic acid sequence of a mutated beta-amyrin synthase (BAS) gene, wherein said mutation is located in a BAS gene: (i) comprising a nucleic acid sequence having at least 80% sequence identity to a nucleic acid sequence of any one of SEQ ID NOs: 1-5 and 38, wherein said nucleic acid sequence encodes a polypeptide that retains BAS activity; (ii) comprising the nucleic acid sequence of any one of SEQ ID NOs: 1-5 and 38; (iii) encoding a polypeptide comprising an amino acid sequence having at least 80% sequence identity to an amino acid sequence of any one of SEQ ID NOs: 6-10, wherein said polypeptide retains BAS activity; and/or (iv) encoding a polypeptide comprising an amino acid sequence of any one of SEQ ID NOs: 6-10. The mutation decreases level or activity of a BAS protein encoded by the BAS gene.

In some embodiments, the nucleic acid sequence of the nucleic acid molecule: (a) has at least 80% identity to a nucleic acid sequence of any one of: (i) SEQ ID NO: 1 consisting of a deletion of nucleotides 4191 through 4195 thereof; (ii) SEQ ID NO: 1 consisting of a G to A substitution of nucleotide 3564 thereof; (iii) SEQ ID NO: 1 consisting of an A to T substitution of nucleotide 374 thereof; (iv) SEQ ID NO: 1 consisting of a deletion of nucleotides 4190 through 4199 thereof; (v) SEQ ID NO: 1 consisting of a deletion of nucleotides 4171 through 4198 thereof; (vi) SEQ ID NO: 1 consisting of a deletion of nucleotides 4187 through 4190 thereof; (vii) SEQ ID NO: 1 consisting of a deletion of nucleotides 4189 through 4198 thereof; (viii) SEQ ID NO: 1 consisting of a deletion of nucleotides 4120-4197 thereof; (ix) SEQ ID NO: 1 consisting of a deletion of nucleotides 4187 through 4191 thereof; (x) SEQ ID NO: 1 consisting of a deletion of nucleotides 4188 through 4195 thereof; or (xi) SEQ ID NO: 1 consisting of a deletion of nucleotides 4187 through 4194 thereof, (b) comprises the nucleic acid sequence of any one of: (i) SEQ ID NO: 1 consisting of a deletion of nucleotides 4191 through 4195 thereof; (ii) SEQ ID NO: 1 consisting of a G to A substitution of nucleotide 3564 thereof; (iii) SEQ ID NO: 1 consisting of an A to T substitution of nucleotide 374 thereof; (iv) SEQ ID NO: 1 consisting of a deletion of nucleotides 4190 through 4199 thereof; (v) SEQ ID NO: 1 consisting of a deletion of nucleotides 4171 through 4198 thereof; (vi) SEQ ID NO: 1 consisting of a deletion of nucleotides 4187 through 4190 thereof; (vii) SEQ ID NO: 1 consisting of a deletion of nucleotides 4189 through 4198 thereof; (viii) SEQ ID NO: 1 consisting of a deletion of nucleotides 4120 through 4197 thereof; (ix) SEQ ID NO: 1 consisting of a deletion of nucleotides 4187 through 4191 thereof; (x) SEQ ID NO: 1 consisting of a deletion of nucleotides 4188 through 4195 thereof; (xi) SEQ ID NO: 1 consisting of a deletion of nucleotides 4187 through 4194 thereof; or (xii) SEQ ID NO: 1 consisting of a deletion of nucleotides 4191 through 4195 thereof, and/or (c) encodes a polynucleotide comprising (i) an amino acid sequence having at least 80% sequence identity to an amino acid sequence of SEQ ID NO: 6 with a G to E substitution of amino acid 220 or an R to W substitution of amino acid 100 or (ii) an amino acid sequence of SEQ ID NO: 6 with a G to E substitution of amino acid 220 or an R to W substitution of amino acid 100. In some embodiments, the nucleic acid sequence of the nucleic acid molecule: (a) has at least 80% identity to a nucleic acid sequence of (i) SEQ ID NO: 38 consisting of a G to A substitution of nucleotide 3750 thereof or (ii) SEQ ID NO: 38 consisting of an A to T substitution of nucleotide 560 thereof; and/or (b) comprises the nucleic acid sequence of any one of (i) SEQ ID NO: 38 consisting of a G to A substitution of nucleotide 3750 thereof or (ii) SEQ ID NO: 38 consisting of an A to T substitution of nucleotide 560 thereof.

In one aspect, the present disclosure provides a DNA construct comprising, in operable linkage: (i) a promoter that is functional in a plant cell; and (ii) the nucleic acid molecule provided herein.

In one aspect, the present disclosure provides a cell comprising the nucleic acid molecule or the DNA construct provided herein. In some embodiments, the cell is a plant cell.

In one aspect, the present disclosure provides a method of producing a population of low-saponin soybean plants or seeds, said method comprising: a) genotyping a first population of soybean plants or seeds for the presence of at least one low-saponin marker that is within 20 centimorgans of at least one low-saponin quantitative trait locus (QTL) located within a genomic region 132866-141435 of chromosome 7 of a soybean genome; b) selecting from the first population one or more soybean plants or seeds comprising one or more low-saponin alleles having the one or more low-saponin molecular markers; and c) producing a second population of progeny soybean plants or seeds from the selected one or more soybean plants or plants grown from the selected seeds, wherein the second population of progeny soybean plants or seeds comprises the one or more low-saponin alleles having the one or more low-saponin molecular markers, and wherein the second population of progeny soybean plants or seeds comprises low-saponin content relative to a control population.

In some embodiments, said at least one low-saponin QTL is Gm07_137242, Gm07_133425, and/or Gm07_136615.

In some embodiments, said at least one low-saponin QTL comprises a single nucleotide polymorphism (SNP), and said at least one low-saponin marker comprises an allele of the SNP. In some embodiments, the SNP is a T or an A at position 133425 and/or an A or a G at position 136615 of chromosome 7 of the soybean genome, wherein the T at position 133425 or the A at position 136615 of chromosome 7 of the soybean genome is associated with low-saponin content.

In some embodiments, said at least one low-saponin QTL comprises a deletion of at least a portion of a beta-amyrin synthase (BAS) gene or regulatory region thereof, and said at least one low-saponin marker comprises an allele comprising the deletion. In some embodiments, said BAS gene is Glyma.07g001300. In some embodiments, said at least one low-saponin QTL comprises a deletion of a portion of exon 7 of the BAS gene. In some embodiments, said deletion comprises a deletion of positions Gm07_137242-137246.

In some embodiments, genotyping comprises analyzing the SNP or the deletion using an oligonucleotide probe comprising at least 15 nucleotides, wherein the oligonucleotide probe has at least 90% sequence identity to a sequence of the same number of contiguous nucleotides of a sense or antisense DNA strand in a region comprising or adjacent to the SNP or the deletion. In some embodiments, said oligonucleotide probe comprises any one of SEQ ID NOs: 17, 18, 21, and 22.

In some embodiments, the genotyping comprises analyzing the SNP or the deletion using a first primer and a second primer each comprising at least 15 nucleotides, wherein the first primer has at least 90% sequence identity to a sequence of the same number of contiguous nucleotides of a sense DNA strand of a region comprising or adjacent to the SNP, and the second primer has at least 90% sequence identity to a sequence of the same number of contiguous nucleotides of an antisense DNA strand of the region comprising or adjacent to the SNP or the deletion. In some embodiments, the first and second primers comprise any one pair of: (i) nucleic acid sequences of SEQ ID NOs: 13 and 14; (ii) nucleic acid sequences of SEQ ID NOs: 15 and 16; and (iii) nucleic acid sequences of SEQ ID NOs: 19 and 20.

In one aspect, the present disclosure provides a population of low-saponin soybean plants or seeds produced by the method provided herein, wherein said low-saponin population of soybean plants or seeds has a greater frequency of the low-saponin marker than said first population of soybean plants or seeds. In some embodiments, the population of low-saponin soybean plants or seeds comprises total saponin content of from about 0 mg/g to about 0.8 mg/g, and/or DDMP saponin content of from about 0 mg/g to about 0.6 mg/g.

In one aspect, the present disclosure provides a method of introgressing a low-saponin QTL. The method comprises (a) crossing a first soybean plant comprising a low-saponin QTL with a second soybean plant of a different genotype to produce one or more progeny plants or seeds and (b) selecting a progeny plant or seed comprising a low-saponin allele of a polymorphic locus linked to the low-saponin QTL, wherein the polymorphic locus is a chromosomal segment comprising a low-saponin marker within the genomic region 132866-141435 of soybean chromosome 7.

In some embodiments, the low-saponin QTL is Gm07_137242, Gm07_133425, or Gm07_136615.

In some embodiments, said low-saponin QTL comprises an SNP marker. In some embodiments, the SNP is a T or an A at position 133425 and/or an A or a G at position 136615 of chromosome 7 of the soybean genome, wherein the T at position 133425 or the A at position 136615 of chromosome 7 of the soybean genome is associated with low-saponin content.

In some embodiments, low-saponin QTL comprises a deletion marker, wherein the deletion comprises a deletion of at least a portion of a beta-amyrin synthase (BAS) gene or regulatory region thereof. In some embodiments, said BAS gene is Glyma.07g001300. In some embodiments, said low-saponin QTL comprises a deletion of a portion of exon 7 of the BAS gene. In some embodiments, said deletion is a deletion of positions Gm07_137242-137246.

In one aspect, the present disclosure provides a nucleic acid molecule for detecting a low-saponin molecular marker in soybean DNA, the nucleic acid molecule comprising at least 15 nucleotides, wherein the nucleic acid molecule has at least 90% sequence identity to a sequence of the same number of contiguous nucleotides of a sense or antisense DNA strand in a region comprising or adjacent to the low-saponin molecular marker. In some embodiments, the low-saponin molecular marker is a SNP marker, and wherein the SNP marker is a T or an A at position 133425 and/or an A or a G at position 136615 of chromosome 7 of the soybean genome, wherein the T at position 133425 or the A at position 136615 of chromosome 7 of the soybean genome is associated with low-saponin content. In some embodiments, the low-saponin molecular marker is a deletion marker, and wherein the deletion maker is a deletion of positions Gm07_137242-137246. In some embodiments, said nucleic acid molecule comprises any one of SEQ ID NOs: 17, 18, 21, and 22. In some embodiments, the nucleic acid molecule provided herein further comprises a detectable label. In some embodiments, said detectable label is a radioactive label or a fluorescent label.

### BRIEF DESCRIPTION OF THE DRAWINGS

FIG. 1 depicts exemplary biosynthetic pathway of triterpenoids including saponins.

FIG. 2A depicts an expression profile of soybean BAS gene copies GmBAS1 (Glyma.07g001300), GmBAS2 (Glyma.08g225800), GmBAS3 (Glyma.03g121300), GmBAS4 (Glyma.03g121500), and GmBAS5 (Glyma.15g101800) in various tissues of soybean based on data available from Phytozome. FIG. 2B depicts an expression profile of BAS gene copies (GmBAS1, GmBAS2, GmBAS3, GmBAS4, and GmBAS5) in various tissues of soybean based on data available from Soybase. FPKM and RPKM stand for fragments per kilobase of exon per million reads and reads per kilobase million, respectively.

FIG. 3 depicts alignment and specificity of GmBAS1 guide RNA 6 to soybean BAS gene copies GmBASI-GmBAS5. MM# stands for the number of mismatched bases. The mismatched bases are underlined.

FIG. 4 shows partial nucleic acid sequences of the Agrobacterium-transformed T0 plants with mutations (deletions) around the targeting site of guide RNA 6 in exon 7 of GmBAS1. The underlined (with solid line) sequence in the WT plant sequence shows the targeting sequence of guide RNA 6. The sequence underlined with dotted line represents protospacer adjacent motif (PAM) sequence for recognition by a nuclease.

FIG. 5 depicts saponin content in BAS1 mutant and control soybean seeds. The left panel depicts total saponin content in control, GmBAS1 R100W mutant, and GmBAS1 G220E mutant soybean seeds. The middle panel depicts DDMP saponin content in control, GmBAS1 R100W mutant, and GmBAS1 G220E mutant soybean seeds. “ND” stands for not detectable. The right panel depicts total saponin content in control and Plant I (having a 5 bp deletion in GmBAS1).

FIG. 6 schematically depicts the GmBAS1 gene and the location of the R100W, G220E, and 5 bp mutations. The shaded boxes in the first row indicates exons in the GmBAS1 gene.

### DETAILED DESCRIPTION OF THE INVENTION

The present disclosure now will be described more fully hereinafter. The disclosure may be embodied in many different forms and should not be construed as limited to the aspects set forth herein; rather, these aspects are provided so that this disclosure will satisfy applicable legal requirements.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous.

A plant refers to a whole plant, any part thereof, or a cell or tissue culture derived from a plant, comprising any of: whole plants, plant components or organs (e.g., leaves, stems, roots, embryos, pollen, ovules, seeds, leaves, flowers, branches, fruit, pulp, juice, kernels, ears, cobs, husks, stalks, root tips, anthers, etc.), plant tissues, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants are also included within the scope of the invention.

As used herein, a “subject plant or plant cell” is one in which genetic alteration, such as a mutation, has been effected as to a gene of interest, or is a plant or plant cell which is descended from a plant or cell so altered and which comprises the alteration. As used herein, the term “mutated” or “genetically modified” or “transgenic” or “transformed” or “edited” plants, plant cells, plant tissues, plant parts or seeds refers plants, plant cells, plant tissues, plant parts or seeds that have been mutated by the methods of the present disclosure to include one or more mutations (e.g., insertions, substitutions, and/or deletions) in the genomic sequence.

As used herein, a “control plant” or “control plant part” or “control cell” or “control seed” refers to a plant or plant part or plant cell or seed that has not been subject to the methods and compositions described herein. A “control” or “control plant” or “control plant part” or “control cell” or “control seed” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli (e.g., sucrose) that would induce expression of the gene of interest; or (e) the subject plant or plant cell itself, under conditions in which the gene of interest is not expressed. In certain instances, a control plant of the present disclosure is grown under the same environmental conditions (e.g., same or similar temperature, humidity, air quality, soil quality, water quality, and/or pH conditions) as a subject plant described herein. Similarly, a control protein or control protein composition can refer to a protein or protein composition that is isolated or derived from a control plant. In specific embodiments, a control plant, plant part, or plant cell is a plant cell that does not have a mutated nucleotide sequence in a BAS gene or a regulatory region of a BAS gene.

Plant cells possess nuclear, plastid, and mitochondrial genomes. The compositions and methods of the present invention may be used to modify the sequence of the nuclear, plastid, and/or mitochondrial genome, or may be used to modulate the expression of a gene or genes encoded by the nuclear, plastid, and/or mitochondrial genome. Accordingly, by “chromosome” or “chromosomal” is intended the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell.

As used herein, the term “gene” or “coding sequence”, herein used interchangeably, refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the term a “nucleic acid”, used interchangeably with a “nucleotide”, refers to a molecule consisting of a nucleoside and a phosphate that serves as a component of DNA or RNA. For instance, nucleic acids include adenine, guanine, cytosine, uracil, and thymine.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNAtranscription factor interactions, RNA-ribosome interactions, gRNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Accordingly, “plant with mutation” or “plant part with mutation” or “plant cell with mutation” or “plant genome with mutation” refers to a plant or plant part or plant cell or plant genome that contains a mutation (e.g., an insertion, a substitution, or a deletion) described in the present disclosure, such as a mutation in the nucleic acid sequence of a BAS gene or a regulatory region of a BAS gene. For example, as used herein, a plant, plant part or plant cell with mutation may refer to a plant, plant part or plant cell in which, or in an ancestor of which, at least one BAS gene or a regulatory region of the BAS gene has been deliberately mutated such that the plant, plant part or plant cell expresses a mutated (e.g., truncated) BAS protein or have a reduced expression level of the BAS gene or BAS protein. The mutated BAS protein can have altered function, e.g., reduced function or loss-of-function, compared to a wild-type, or control, BAS protein comprising no mutation.

“Genome editing” or “gene editing” as used herein refers to a type of genetic engineering by which one or more mutations (e.g., insertions, substitutions, deletions, modifications) are introduced at a specific location of the genome.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

An expression construct can permit transcription of a particular nucleic acid sequence in a host cell (e.g., a bacterial cell or a plant cell). An expression cassette may be part of a plasmid, viral genome, or nucleic acid fragment. Typically, an expression cassette includes a polynucleotide to be transcribed, operably linked to a promoter. “Operably linked” is intended to mean a functional linkage between two or more elements. For example, an operable linkage between a promoter of the present invention and a heterologous nucleotide is a functional link that allows for expression of the heterologous nucleic acid molecule. Operably linked elements may be contiguous or noncontiguous. When used to refer to the joining of two protein coding regions, by operably linked is intended that the coding regions are in the same reading frame. The cassette may additionally contain at least one additional gene to be co-transformed into the plant. Alternatively, the additional gene(s) can be provided on multiple expression cassettes or DNA constructs. The expression cassette may additionally contain selectable marker genes. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, “function” of a gene, a peptide, a protein, or a molecule refers to activity of a gene, a peptide, a protein, or a molecule.

“Introduced” in the context of inserting a nucleic acid molecule (e.g., a recombinant DNA construct) into a cell, means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

As used herein with respect to a parameter, the term “decreased” or “decreasing” or “decrease” or “reduced” or “reducing” or “reduce” or “lower” or “loss” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “decreased”, “reduced”, and the like encompass both a partial reduction and a complete reduction compared to a control.

As used herein with respect to a parameter, the term “increased” or “increasing” or “increase” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, 120%, 150%, 200%, 300%, 400%, 500%, or more) positive change in the parameter from a comparison control, e.g., an established normal or reference level of the parameter, or an established standard control. Accordingly, the terms “increased”, “increase”, and the like encompass a mild, moderate, or significant increase compared to a control.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleic acid sequence (cDNA), a genomic polynucleic acid sequence and/or a composite polynucleic acid sequences (e.g., a combination of the above).

The term “isolated” refers to at least partially separated from the natural environment e.g., from a plant cell.

As used herein, the term “expression” or “expressing” refers to the transcription and/or translation of a particular nucleic acid sequence driven by a promoter.

As used herein, the terms “exogenous” or “heterologous” in reference to a nucleic acid sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, by “endogenous” in reference to a gene or nucleic acid sequence or protein is intended a gene or nucleic acid sequence or protein that is naturally comprised within or expressed by a cell. Endogenous genes can include genes that naturally occur in the cell of a plant, but that have been modified in the genome of the cell without insertion or replacement of a heterologous gene that is from another plant species or another location within the genome of the modified cell.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

“Homolog” or “homologous sequence” may refer to both orthologous and paralogous sequences. Paralogous sequence relates to gene-duplications within the genome of a species. Orthologous sequence relates to homologous genes in different organisms due to ancestral relationship. Thus, orthologs are evolutionary counterparts derived from a single ancestral gene in the last common ancestor of given two species and therefore have great likelihood of having the same function. One option to identify homologs (e.g., orthologs) in monocot plant species is by performing a reciprocal BLAST search. This may be done by a first blast involving blasting the sequence-of-interest against any sequence database, such as the publicly available NCBI database which may be found at: ncbi.nlm.nih.gov. If orthologs in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An ortholog is identified when the sequence resulting in the highest score (best hit) in the first blast identifies in the second blast the query sequence (the original sequence-of-interest) as the best hit. Using the same rational a paralog (homolog to a gene in the same organism) is found. In case of large sequence families, the ClustalW program may be used [ebi.ac.uk/Tools/clustalw2/index.html], followed by a neighbor-joining tree (wikipedia.org/wiki/Neighbor-joining) which helps visualizing the clustering.

In some embodiments, the term “homolog” as used herein, refers to functional homologs of genes. A functional homolog is a gene encoding a polypeptide that has sequence similarity to a polypeptide encoded by a reference gene, and the polypeptide encoded by the homolog carries out one or more of the biochemical or physiological function(s) of the polypeptide encoded by the reference gene. In general, it is preferred that functional homologs and/or polypeptides encoded by functional homologs share at least some degree of sequence identity with the reference gene or polypeptide encoded by the reference gene.

Homology (e.g., percent homology, sequence identity+sequence similarity) can be determined using any homology comparison software computing a pairwise sequence alignment.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol.266:131-141; Altschul et al. (1997), Nucleic Acids Res. 25:3389-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=-11; gap extension penalty=-1; and scoring matrix=BLOSUM62. As used herein, percent similarity of two nucleic acid sequences is the score based upon the following parameters for the BLASTn algorithm: word size=11; gap opening penalty=-5; gap extension penalty=-2; match reward=1; and mismatch penalty=-3. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Proc Natl Acad Sci 89:10915-9 (1992)). Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence. According to some embodiments, the homology is a global homology, e.g., a homology over the entire amino acid or nucleic acid sequences of the invention and not over portions thereof. The degree of homology or identity between two or more sequences can be determined using various known sequence comparison tools which are described in WO2014/102774.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “population” refers to a set comprising any number, including one, of individuals, objects, or data from which samples are taken for evaluation, e.g., estimating quantitative trait locus (QTL) effects and/or disease tolerance. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silico representations of plants. The member of a population need not be identical to the population members selected for use in subsequent cycles of analyses, nor does it need to be identical to those population members ultimately selected to obtain a final progeny of plants. Often, a plant population is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

As used herein, the term “crop performance” is used synonymously with “plant performance” and refers to of how well a plant grows under a set of environmental conditions and cultivation practices. Crop performance can be measured by any metric a user associates with a crop’s productivity (e.g., yield), appearance and/or robustness (e.g., color, morphology, height, biomass, maturation rate, etc.), product quality (e.g., fiber lint percent, fiber quality, seed protein content, seed carbohydrate content, etc.), cost of goods sold (e.g., the cost of creating a seed, plant, or plant product in a commercial, research, or industrial setting) and/or a plant’s tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop’s commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants. A “pathogen” as used herein refers to a microbe that causes disease or harmful effects on plant health.

A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a pesticide is any substance that reduces the survivability and/or reproduction of a pest, e.g. fungicides, bactericides, insecticides, herbicides, and other toxins.

“Tolerance” or “improved tolerance” in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different soybean cultivar) grown in similar circumstances. As used in the art, “tolerance” is sometimes used interchangeably with “resistance”, although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

“Yield” as used herein is defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance, photosynthetic carbon assimilation rates, and early vigor may also be important factors in determining yield. Optimizing the abovementioned factors may therefore contribute to increasing crop yield. Yield can be measured and expressed by any means known in the art. In specific embodiments, yield is measured by seed weight or volume in a given harvest area.

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant’s growth, development and/or performance, including agents that affect other organisms in the plant’s environment when those effects subsequently alter a plant’s performance, growth, and/or development (e.g. an insecticide that kills plant pathogens in the plant’s environment, thereby improving the ability of the plant to tolerate the insect’s presence). Agriculture treatment agents also include a broad range of chemicals and/or biological substances that are applied to seeds, in which case they are commonly referred to as seed treatments and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g. microencapsulation). In certain embodiments, the agriculture treatment agent comprises a nanoparticle and/or the application of the agriculture treatment agent comprises the use of nanotechnology. In some embodiments, the plants described herein can grow in the presence of one or more agricultural treatment agents. For example, the plants described herein can have a decreased saponin content and can grow in the presence of commonly used herbicides.

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

All publications, patent applications, patents, and other references mentioned herein are incorporated by reference herein in their entirety.

### II. Overview of the Invention

Improving the flavor characteristics of plants, plant parts, and plant products would offer commercial advantages in the growing plant-based food market. For example, to achieve broader consumer acceptance, it is necessary to improve off flavors inherent to plant meal, including soybean meal-the leading source of protein in plant-based foods-commonly described as grassy, stale, or bitter. Such off-flavors in plants, plant parts, and plant-based food and beverage products can be caused by secondary metabolites, such as saponin, contained therein. Saponin can also cause various pathology in aquaculture fish species when contained in aquaculture feeds.

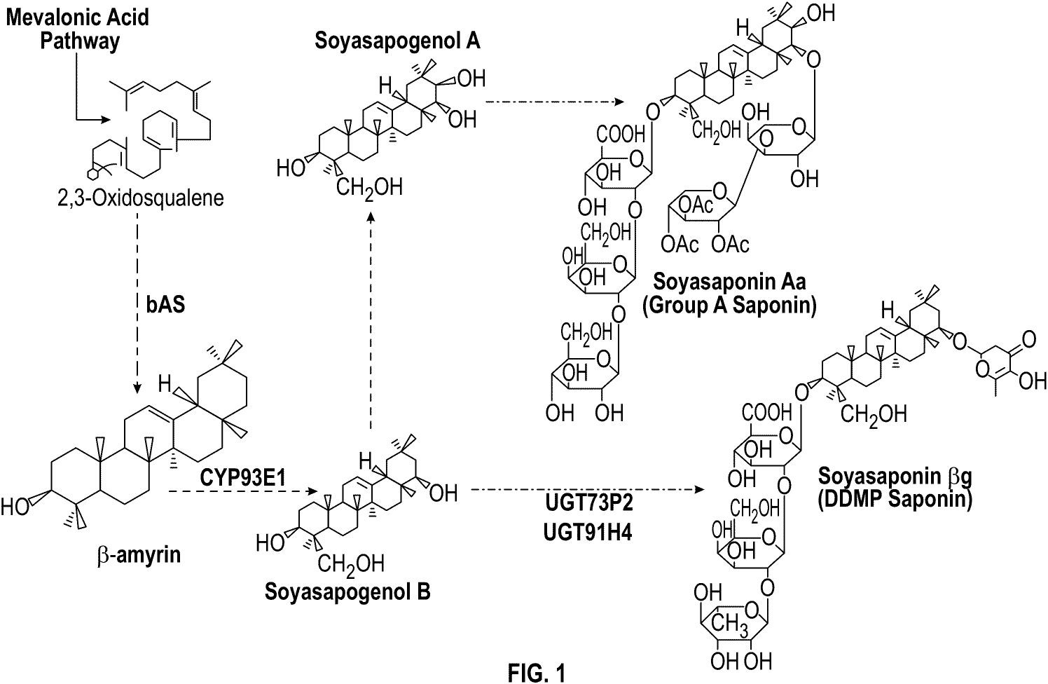

As depicted in FIG. 1, beta-amyrin synthase (BAS) catalyzes cyclization of 2, 3-oxidosqualene to beta-amyrin, a key step in saponin synthesis in plants (Sawai & Saito 2011 Front Plant Sci 2;25:1-8). Mixed-function triterpene synthase (MAS) enzymes catalyze the synthesis of not only beta-amyrin but also other triterpenes. BAS and MAS enzymes have been identified in various plant species, including Glycine max, Glycyrrhiza glabra, Lotus japonicus, Pisum sativum, Medicago truncatula, Arabidopsis thaliana, Vitis vinifera, Vitis riparia, Lactuca sativa, Nicotiana sylvestris, Panax ginseng, and Eucalyptus grandis. Each plant can have one, or more than one, genes encoding BAS enzymes. Beta-amyrin synthesized by BAS enzymes then undergoes modifications including P450-catalyzed oxidation and UDP-dependent glycosyl-transferase (UGT)-catalyzed glycosylation, and is converted to saponins. Reducing BAS activity can reduce saponin production or content in plants or plant parts.

Disclosed herein are plants or plant parts comprising a genetic mutation that decreases the BAS activity compared to a control plant or plant part, as well as methods for making the plants or plant parts. Such plants or plant parts can have one or more insertions, substitutions, or deletions in at least one native BAS gene or homolog thereof or in its regulatory region. The plants or plant parts can have reduced expression level of the BAS gene or homolog thereof, reduced level or activity of the BAS protein encoded by the BAS gene or homolog thereof, reduced saponin content, and/or improved flavor characteristics compared to a plant or plant part without the mutation. The present disclosure also provides compositions and methods for producing plants plant parts, or a population of plants or plant parts with reduced saponin content by introducing a genetic mutation that decreases BAS activity. The methods disclosed herein can include introducing one or more insertions, substitutions, or deletions in at least one BAS gene or homolog thereof or in its regulatory region in the genome of a plant, plant part, or plant cell, such that an expression level of the BAS gene or homolog thereof is reduced, level or activity of a BAS protein encoded by the BAS gene or homolog thereof is reduced, saponin content is reduced, and/or flavor characteristics are improved in the plant, plant part, or plant cell compared to a plant, plant part, or plant cell without the mutation. The methods of the present disclosure can include introducing editing reagents (e.g., nuclease, guide RNA) into the plants or plant parts to introduce a mutation in at least one native BAS gene or homolog thereof or in its regulatory region. The plants, plant parts, or plant products, such as plant protein composition or food and beverage products comprising the plant composition of the present disclosure, including those produced using the methods disclosed herein, can have reduced BAS activity, reduced saponin content, and/or improved flavor characteristics. Also disclosed herein are a population of plants or plant parts (e.g., seeds) having decreased BAS activity, a decreased saponin content, and/or improved flavor characteristics compared to a control population, and protein compositions, or food and beverage products produced from the population of plants or plant parts of the present disclosure. Further provided herein are nucleic acid molecules comprising a mutated BAS gene, a DNA construct comprising such nucleic acid molecule operably linked to a promoter, and cells comprising the nucleic acid molecule or the DNA construct of the present disclosure.

### III. Plants With Altered Saponin Content

Plants and plant parts are provided herein having altered (e.g., decreased) beta-amyrin synthase (BAS) level or activity as compared to a control plant or plant part. The plants or plant parts described herein having altered BAS level or activity can comprise a genetic mutation that alters (e.g., decreases) BAS level or activity, altered (e.g., decreased) expression levels of at least one BAS gene encoding BAS protein, altered (e.g., decreased) BAS protein levels or activity, altered (e.g., decreased) beta-amyrin levels, altered (e.g., decreased) saponin content, and/or altered (e.g., improved) flavor characteristics compared to a control plant or plant part.

Also provided herein is a population of plants and plant parts comprising the plants and plant parts described herein having altered (e.g., decreased) BAS level or activity. In such population of plants or plant parts, having altered BAS level or activity relative to a control population, not all individual plants or plant parts need to have altered (e.g., decreased) BAS level or activity, genetic mutation that cause altered (e.g., decreased) BAS level or activity, or phenotypes caused by the altered (e.g., decreased) BAS activity (e.g., decreased saponin content, improved flavor characteristics). In specific embodiments at least 80%, 81%, 82%, 83%, 84%, 85%, 86%, 87%, 88%, 89%, 90%, 91%, 92%, 93%, 94%, 95%, 96%, 97%, 98%, 99%, or more plants within a given plant population have a mutation that alters the BAS level or activity.

A plant or plant part of the present disclosure can be a legume, i.e., a plant belonging to the family Fabaceae (or Leguminosae), or a part (e.g., fruit or seed) of such a plant. When used as a dry grain, the seed of a legume is also called a pulse. Examples of legume include, without limitation, soybean (Glycine max), beans (Phaseolus spp.), common bean (Phaseolus vulgaris), fava bean (Vicia faba), mung bean (Vigna radiata), pea (Pisum sativum), chickpea (Cicer arietinum), peanut (Arachis hypogaea), lentils (Lens culinaris, Lens esculenta), lupins (Lupinus spp.), white lupin (Lupinus albus), mesquite (Prosopis spp.), carob (Ceratonia siliqua.), tamarind (Tamarindus indica), alfalfa (Medicago sativa), barrel medic (Medicago truncatula), birdsfood trefoil (Lotus japonicus), licorice (Glycyrrhiza glabra), and clover (Trifolium spp.). For example, a plant or plant part of the present disclosure can be Glycine max or a part of Glycine max. Additionally, a plant or plant part of the present disclosure can be a crop plant or part of a crop plant, including legumes. Examples of crop plants include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana spp., e.g., Nicotiana tabacum, Nicotiana sylvestris), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), grapes (Vitis vinifera, Vitis riparia), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, a plant or plant part of the present disclosure can be an oilseed plant (e.g., canola (Brassica napus), cotton (Gossypium sp.), camelina (Camelina sativa) and sunflower (Helianthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), or hemp (Cannabis sativa).

### A. Plants With Altered Level or Activity of Beta-Amyrin Synthase

Provided herein are plants and plant parts comprising altered (e.g., decreased) beta-amyrin synthase (BAS) activity compared to a control plant or plant part. As used herein, “beta-amyrin synthase (BAS) activity” refers to the ability of an enzyme (e.g., BAS enzyme) to catalyze production of beta-amyrin, e.g., by cyclizing 2, 3-oxidosqualene to beta-amyrin. In particular aspects, plants and plant parts (e.g., seeds, fruits) disclosed herein have a genetic mutation that alters (e.g., decreases) the beta-amyrin synthase activity. Also provided herein is a population of plants or plant parts (e.g., seeds) comprising altered (e.g., decreased) BAS activity compared to a control population provided herein.

The genetic mutation that alters (e.g., decreases) the BAS activity in the plants and plant parts provided herein can comprise one or more insertions, substitutions, or deletions in at least one native BAS gene or homolog thereof, or in a regulatory region of at least one native BAS gene or homolog thereof. The genetic mutation that alters (e.g., decreases) the BAS activity can be located in at least one native BAS gene or homolog thereof; in a regulatory region of the native BAS gene or homolog thereof; a coding region, a non-coding region, or a regulatory region of any other gene; or at any other site in the genome of the plant or plant part. A “native” gene, as used herein, refers to any gene having a wild-type nucleic acid sequence, e.g., a nucleic acid sequence that can be found in the genome of a plant existing in nature, and need not naturally occur within the plant, plant part, or plant cell comprising such native gene. For example, a transgenic BAS gene located at a genomic site or in a plant in a non-naturally occurring matter is a “native” BAS gene if its nucleic acid sequence can be found in a plant existing in nature. A “regulatory region” of a gene, as used herein, refers to the region of a genome that controls expression of the gene. A regulatory region of a gene can include a genomic site where a RNA polymerase, a transcription factor, or other transcription modulators bind and interact to control mRNA synthesis of the gene, such as promoter regions, binding sites for transcription modulator proteins, and other genomic regions that contribute to regulation of transcription of the gene. A regulatory region of the gene can be located in the 5′ untranslated region of the gene.

A control plant or plant part can be a plant or plant part to which a mutation provided herein has not been introduced, e.g., by methods of the present disclosure. Thus, a control plant or plant part (e.g., seeds, fruit) may express a native (e.g., wild-type) BAS gene endogenously or transgenically. A control plant of the present disclosure may be grown under the same environmental conditions (e.g., same or similar temperature, humidity, air quality, soil quality, water quality, and/or pH conditions) as a plant with the mutation described herein. A plant, plant part (e.g., seeds, fruit), or a population of plants or plant parts of the present disclosure may have altered (e.g., decreased) expression levels of at least one BAS gene or homolog thereof, altered (e.g., decreased) BAS protein level or activity, altered (e.g., decreased) beta-amyrin levels, altered (e.g., decreased) saponin content, and/or altered (e.g., improved) flavor characteristics as compared to a control plant, plant part, or population when the plant, plant part, or population of plants or plant parts of the present disclosure is grown under the same environmental conditions as the control plant or plant part.

### (I) Plants With One or More Mutations in at Least One BAS Gene or its Regulatory Region

In some aspects, the plants and plant parts of the present disclosure comprise decreased BAS activity and a genetic mutation that decreases the BAS activity. The genetic mutation can comprise one or more (e.g., about 1, 2, 3, 4, 5, 6, 7, 8, 9, 10, 11, 12, 13, 14, 15, 16, 17, 18, 19, 20, or more) insertions, substitutions, and/or deletions in at least one native BAS gene or homolog thereof and/or in a regulatory region of said at least one native BAS gene or homolog thereof in a genome of said plant or plant part. A plant or plant part described herein can comprise 1-10, 1-5, 2-9, 2-8, 2-7, 2-6, 2-5, 3-5, 4-5 (e.g., 1, 2, 3, 4, 5, 6, 7, 8, 9, or 10) copies of BAS gene, e.g., BAS1, BAS2, BAS3, BAS4, and BAS5 genes, each encoding a BAS protein. In particular, a plant or plant part described herein can comprise at least 2 genes encoding a BAS protein, such as 2, 3, 4, 5, 6, 7, 8, 9, or 10 genes that have less than 100% (e.g., less than 100%, 99%, 98%, 97%, 96%, 95%, 94%, 93%, 92%, 91%, 90%, 89%, 88%, 87%, 86%, or 85%) sequence identity to one another. The plant or plant part described herein can comprise one or more (e.g., about 1, 2, 3, 4, 5, 6, 7, 8, 9, 10, 11, 12, 13, 14, 15, 16, 17, 18, 19, 20, or more) insertions, substitutions, and/or deletions: in one BAS gene or homolog; in a regulatory region of one BAS gene or homolog; in more than one (e.g., 2, 3, 4, 5, 6, 7, 8, 9, 10), but not all BAS genes or homologs; in regulatory regions of more than one (e.g., 2, 3, 4, 5, 6, 7, 8, 9, 10), but not all BAS genes or homologs; in all BAS genes or homologs; and/or in regulatory regions of all BAS genes or homologs in the plant or plant part.

The mutation that decreases the BAS activity can be located in one or more (e.g., one, more than one but not all, or all) Glycine max BAS genes, such as a Glycine max BAS1 gene, a Glycine max BAS2 gene, a Glycine max BAS3 gene, a Glycine max BAS4 gene, a Glycine max BAS5 gene and/or a regulatory region of such one or more Glycine max BAS genes. In some embodiments, the mutation is located in a Glycine max BAS1 gene and/or a regulatory region of the Glycine max BAS1 gene. In some embodiments, the mutation that decreases the BAS activity can be located in a BAS gene or homolog thereof comprising a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of any one of SEQ ID NOs: 1-5 and 38 and encoding a polypeptide that retains BAS activity, for example the nucleic acid sequence of any one of SEQ ID NOs: 1-5 and 38; and/or a regulatory region of the BAS gene or homolog thereof comprising such nucleic acid sequence. Additionally, the mutation can be located in a BAS gene or homolog thereof encoding a polypeptide comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of any one of SEQ ID NOs: 6-10 and retaining BAS activity, for example a polypeptide comprising the amino acid sequence of any one of SEQ ID NOs: 6-10; and/or a regulatory region of the BAS gene or homolog thereof encoding such polypeptide. In specific embodiments, the mutation that decreases the BAS activity is located in a BAS gene or homolog thereof comprising a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence of SEQ ID NO: 1 or 38 and encoding a polypeptide that retains BAS activity, for example the nucleic acid sequence of SEQ ID NO: 1 or 38, and/or a regulatory region of the BAS gene or homolog thereof comprising such nucleic acid sequence. Additionally, the mutation can be located in a BAS gene or homolog thereof encoding a polypeptide comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to an amino acid sequence of SEQ ID NO: 6 and retaining BAS activity, for example a polypeptide comprising an amino acid sequence of SEQ ID NO: 6; and/or a regulatory region of the BAS gene or homolog thereof encoding such polypeptide.

In the plant or plant part provided herein comprising a mutation that decreases the BAS activity, at least one (e.g., about 1, 2, 3, 4, 5, 6, 7, 8, 9, 10, 11, 12, 13, 14, 15, 16, 17, 18, 19, 20, or more) insertion, substitution, or deletion can be located in a nucleic acid region of exon 10 or upstream of exon 10 of a Glycine max BAS1 gene. In some embodiments, at least one (e.g., about 1, 2, 3, 4, 5, 6, 7, 8, 9, 10, 11, 12, 13, 14, 15, 16, 17, 18, 19, 20, or more) insertion, substitution, or deletion can be at least partially in a nucleic acid region of exon 7 of the Glycine max BAS1 gene. As used herein, where an insertion, a substitution, or a deletion is “at least partially” in a certain nucleotide region, the whole part of the insertion, substitution, or deletion can be within the certain nucleotide region, or alternatively, can span across the certain nucleotide region and a region outside the nucleotide region. For instance, where an insertion, a substitution, or a deletion is at least partially in an exon, the whole part of the insertion, the substitution, or the deletion can be within the exon, or can span across the exon and a region (e.g., an intron, a regulatory region) upstream or downstream of the exon.

In some embodiments, the plant or plant part of the present disclosure comprises a deletion of about 4-78 nucleotides at least partially in the nucleic acid region of exon 7 of the Glycine max BAS1 gene. For example, the plant or plant part of the present disclosure can comprise (i) a deletion of nucleotides 4191 through 4195 of SEQ ID NO: 1 in the Glycine max BAS1 gene (at chr07:137242 to 137246 in the Glycine max BAS1 gene), (ii) a deletion of nucleotides 4190 through 4199 of SEQ ID NO: 1 in the Glycine max BAS1 gene, (iii) a deletion of nucleotides 4171 through 4198 of SEQ ID NO: 1 in the Glycine max BAS1 gene, (iv) a deletion of nucleotides 4187 through 4190 of SEQ ID NO: 1 in the Glycine max BAS1 gene, (v) a deletion of nucleotides 4189 through 4198 of SEQ ID NO: 1 in the Glycine max BAS1 gene, (vi) a deletion of nucleotides 4120 through 4197 of SEQ ID NO: 1 in the Glycine max BAS1 gene, (vii) a deletion of nucleotides 4187 through 4191 of SEQ ID NO: 1 in the Glycine max BAS1 gene, (viii) a deletion of nucleotides 4188 through 4195 of SEQ ID NO: 1 in the Glycine max BAS1 gene, and/or (ix) a deletion of nucleotides 4187 through 4194 of SEQ ID NO: 1 in the Glycine max BAS1 gene.

In some embodiments, the plant or plant part of the present disclosure comprises a substitution in the nucleic acid region of exon 2 and/or 4 of the Glycine max BAS1 gene. For example, the plant or plant part of the present disclosure can comprise a G to E substitution of amino acid 220 of SEQ ID NO: 6 of the BAS protein, or a genetic mutation that results in such substitution, for example a G to A substitution of nucleotide 3564 of SEQ ID NO: 1 or a G to A substitution of nucleotide 3750 of SEQ ID NO: 38 (at chr07:136615 of the Glycine max BAS1 gene). Additionally or alternatively, the plant or plant part of the present disclosure can comprise an R to W substitution of amino acid 100 of SEQ ID NO: 6 of the BAS protein, or a genetic mutation in such substitution, for example an A to T substitution of nucleotide 374 of SEQ ID NO: 1 or an A to T substitution of nucleotide 560 of SEQ ID NO: 38 (at chr07:133425 of the Glycine max BAS1 gene).

The mutation that decreases the BAS activity in the plant or plant part disclosed herein can comprise an out-of-frame mutation of at least one (e.g., one, more than one but not all, or all) BAS gene or homolog thereof. Alternatively, the mutation in the plant or plant part can comprise an in-frame mutation, such as a missense mutation, or a nonsense mutation of at least one (e.g., one, more than one but not all, or all) BAS gene or homolog thereof.

The plants or plant parts described herein can comprise a mutation that decreases the BAS activity, e.g., one or more (e.g., about 1, 2, 3, 4, 5, 6, 7, 8, 9, 10, 11, 12, 13, 14, 15, 16, 17, 18, 19, 20, or more) insertions, substitutions, and/or deletions in a regulatory region of at least one (e.g., one, more than one but not all, or all) BAS gene. The regulatory region having the mutation can comprise a promoter region, a binding site (e.g., an enhancer sequence) for a transcription modulator protein (e.g., transcription factor), or other genomic regions that contribute to regulation of transcription of the BAS gene. One or more insertions, substitutions, and/or deletions can be introduced into a promoter region, a transcription modulator protein (e.g., transcription factor) binding site, or other regulatory regions of at least one (e.g., one, more than one but not all, or all) BAS gene to confer to the plant or plant part an altered (e.g., reduced) transcription activity of the BAS gene.

In some embodiments, the mutation is in a promoter region of at least one (e.g., one, more than one but not all, or all) BAS gene. As used herein, a “promoter” refers to an upstream regulatory region of DNA prior to the ATG of a native gene, having a transcription initiation activity (e.g., function) for said gene and other downstream genes. “Transcription initiation” as used herein refers to a phase or a process during which the first nucleotides in the RNA chain are synthesized. It is a multistep process that starts with formation of a complex between a RNA polymerase holoenzyme and a DNA template at the promoter, and ends with dissociation of the core polymerase from the promoter after the synthesis of approximately first nine nucleotides. A promoter sequence can include a 5′ untranslated region (5′UTR), including intronic sequences, in addition to a core promoter that contains a TATA box capable of directing RNA polymerase II (pol II) to initiate RNA synthesis at the appropriate transcription initiation site for a particular polynucleotide sequence of interest. A promoter may additionally comprise other recognition sequences positioned upstream of the TATA box, and well as within the 5′UTR intron, which influence the transcription initiation rate. The one or more insertions, substitutions, and/or deletions in the promoter region of the BAS gene can alter the transcription initiation activity of the promoter. For example, the modified promoter can reduce transcription of the operably linked nucleic acid molecule (e.g., the BAS gene), initiate transcription in a developmentally-regulated or temporally-regulated manner, initiate transcription in a cell-specific, cell-preferred, tissue-specific, or tissue-preferred manner, or initiate transcription in an inducible manner. A deletion, a substitution, or an insertion, e.g., introduction of a heterologous promoter sequence, a cis-acting factor, a motif or a partial sequence from any promoter, including those described elsewhere in the present disclosure, can be introduced into the promoter region of the BAS gene to confer an altered (e.g., reduced) transcription initiation function according to the present disclosure. The insertion, substitution, or deletion can comprise insertion, substitution, or deletion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. The substitute can be a cisgenic substitute, a transgenic substitute, or both. The mutation of a promoter region can comprise correction of the promoter sequence by: (i) detection of one or more polymorphism or mutation that enhances the activity of the promoter sequence; and (ii) correction of the promoter sequences by deletion, modification, and/or correction of the polymorphism or mutation. In some embodiments, the mutation is in the upstream region of a promoter region of at least one (e.g., one, more than one but not all, or all) BAS gene.

In some embodiments, a mutation is located in the gene encoding (or regulating expression of) one or more transcription factors that regulates expression of a BAS gene. A “transcription factor” as used herein refers to a protein (other than an RNA polymerase) that regulates transcription of a target gene. A transcription factor has DNA-binding domains to bind to specific genomic sequences such as an enhancer sequence or a promoter sequence. In some instances, a transcription factor binds to a promoter sequence near the transcription initiation site and regulate formation of the transcription initiation complex. A transcription factor can also bind to regulatory sequences, such as enhancer sequences, and modulate transcription of the target gene. The mutation in the gene encoding (or regulating expression of) a transcription factor can modulate expression or function of the transcription factor and reduce expression levels of the BAS gene, e.g., by inhibiting transcription initiation activity of the BAS gene promoter. In some embodiments, the mutation modifies or inserts transcription factor binding sites or enhancer elements that regulates BAS gene expression into the regulatory region of the BAS gene.

In some embodiments, the mutation inserts a part or whole of one or more negative regulatory sequences of the BAS gene into the genome of a plant cell or plant part. The negative regulatory sequence of the gene can be in a cis location or in a trans location. Negative regulatory sequences of the one or more BAS genes can also include upstream open reading frames (uORFs). In some instances, a negative regulatory sequence can be inserted in a region upstream of the BAS gene in order to inhibit the expression and/or function of the gene.

A plant or plant part of the present disclosure can have a genetic mutation that decreases the BAS activity in a gene that is a homolog, ortholog, or variant of a BAS gene disclosed herein and expresses a BAS protein with BAS function, or in a regulatory region of such homolog, ortholog, or variant of a BAS gene. By “orthologs” is intended genes derived from a common ancestral gene and found in different species as a result of speciation. Genes found in different species are considered orthologs when their nucleic acid sequences and/or their encoded protein sequences share at least 75%, 80%, 85%, 90%, 91%, 92%, 93%, 94%, 95%, 96%, 97%, 98%, 99%, or greater sequence identity. Functions of orthologs are often highly conserved among species. Thus, plants or plant parts comprising polynucleotides that have BAS activity and share at least 75% sequence identity to the sequences disclosed herein are encompassed by the present disclosure and can have a genetic mutation that decreases the BAS activity. For example, orthologs of BAS genes disclosed herein include, but are not limited to red clover BAS (Trifolium pratense, NCBI ID: MG492000.1), barrel medic BAS (Medicago truncatula, NCBI ID: AJ430607.1), chickpea BAS (Cicer arietinum, NCBI ID: XM_027335420.1), narrow-leaved blue lupine BAS (Lupinus angustifolius, NCBI ID: XM_019600620.1), pigeon pea BAS (Cajanus cajan, NCBI ID: XM_020370843.2, XM_020370845.2, XM_020370844.2, XM_029273321.1), peanut BAS (Arachis hypogaea, NCBI ID: XM_025789404.2, XM_025789405.2), cowpea BAS (Vigna unguiculata, NCBI ID: XM_028051921.1), adzuki bean BAS (Vigna angularis, NCBI ID: XM_017552062.1), mung bean BAS (Vigna radiata, NCBI ID: XM_022787203.1, XM_022787202.1, XM_022787208.1, XM_022787209.1, XM_022787206.1, XM_022787207.1, XM_022787210.1, XM_014662050.2, XM_014662049.2, XM_022787200.1, XM_022787199.1, XM_022787201.1, XM_022787205.1, XM_022787204.1, XM_022787211.1), pea BAS (Pisum sativum, AB034802.1), and licorice BAS (Glycyrrhiza glabra, AB037203.1).

Variant sequences (e.g., homologs, orthologs) can be isolated by PCR or quantitative PCR. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Variant sequences (e.g., homologs, orthologs) may also be identified by analysis of existing databases of sequenced genomes. In this manner, variant sequences encoding BAS can be identified and used in the methods of the present disclosure. The variant sequences will retain the BAS activity.

In certain instances, mutations in any BAS gene in a plant, plant part, population of plants or plant parts, or plant product (e.g., plant protein composition) can be identified by a diagnostic method described herein. Such diagnostic methods may comprise use of primers for detecting mutation in a BAS gene. For example, the forward primer GATAGTCGTTCATTATGTCAATC (SEQ ID NO: 13) and the reverse primer CACACAACCAATGGTTATG (SEQ ID NO: 14) can be used for detection of mutation in Glycine max BAS1 gene near binding site of GmBAS1 guide RNA 6 (SEQ ID NO: 12) in exon 7, e.g., a deletion of nucleotides 4191 through 4195 of SEQ ID NO: 1 (chr07:137242..137246). The forward primer GACTATAGAAGATGGAGAGGAAATCACAT (SEQ ID NO: 15) and the reverse primer AAGAGAGGACCTGCAATTTGAGC (SEQ ID NO: 16) can be used for detection of mutation in Glycine max BAS1 gene at or near nucleotide 374 of SEQ ID NO: 1 or nucleotide 560 of SEQ ID NO: 38 (chr07:133425). The probes CCGTCAGATGGGG (SEQ ID NO: 17) and GTCAGAAGGGGCG (SEQ ID NO: 18), optionally coupled with a quencher, e.g., minor groove binder (MGB), can be used for detecting an A to T substitution and a wild-type sequence (A), respectively, at nucleotide 374 of SEQ ID NO: 1 or at nucleotide 560 of SEQ ID NO: 38 in the GmBAS1 gene (chr07:133425). The forward primer TAGAGCAAGAAAGTGGATTCGAGA (SEQ ID NO: 19) and the reverse primer CACCGAGTATCTACAAGAGCAAGATC (SEQ ID NO: 20) can be used for detection of mutation in Glycine max BAS1 gene at or near nucleotide 3564 of SEQ ID NO: 1 or nucleotide 3750 of SEQ ID NO: 38 (chr07:136615). The probes TCATGGGAAAAAA (SEQ ID NO: 21) and CTTCATGGGGAAAAA (SEQ ID NO: 22), optionally coupled with a quencher, e.g., minor groove binder (MGB), can be used for detecting a G to A substitution and a wild-type sequence (G), respectively, at nucleotide 3564 of SEQ ID NO: 1 or nucleotide 3750 of SEQ ID NO: 38 in the GmBAS1 gene (chr07:136615).

In certain instances, a kit comprising a set of primers can be used for detecting mutation of BAS genes in plants, plant parts, or plant product (e.g., plant protein composition). For example, a kit comprising the forward primer GATAGTCGTTCATTATGTCAATC (SEQ ID NO: 13) and the reverse primer CACACAACCAATGGTTATG (SEQ ID NO: 14), the forward primer GACTATAGAAGATGGAGAGGAAATCACAT (SEQ ID NO: 15) and the reverse primer AAGAGAGGACCTGCAATTTGAGC (SEQ ID NO: 16), the probes CCGTCAGATGGGG (SEQ ID NO: 17) and/or GTCAGAAGGGGCG (SEQ ID NO: 18), the forward primer TAGAGCAAGAAAGTGGATTCGAGA (SEQ ID NO: 19) and the reverse primer CACCGAGTATCTACAAGAGCAAGATC (SEQ ID NO: 20), and/or the probes TCATGGGAAAAAA (SEQ ID NO: 21) and/or CTTCATGGGGAAAAA (SEQ ID NO: 22) can be used for detection of mutation in BAS1 gene in plants, plant parts, or plant products (e.g., plant protein and/or oil compositions).

In some embodiments, the mutations, e.g., one or more insertions, substitutions, or deletions are integrated into the plant genome and the plant or the plant part is stably transformed. In other embodiments, the one or more mutations are not integrated into the plant genome and wherein the plant or the plant part is transiently transformed.

Also provided herein is a population of plants or plant parts (e.g., seeds) comprising the plants and plant parts having a genetic mutation that decreases the BAS activity described herein.

One or mutations insertions, substitutions, or deletions located in at least one BAS gene or homolog or in a regulatory region of such BAS gene or homolog in the genome of the plant or plant part can reduce the expression levels of the BAS gene or homolog, reduce level or activity of the BAS protein encoded by the BAS gene or homolog, reduce BAS activity in the plant or plant part, reduce saponin content in the plant or plant part, and/or improve flavor characteristics in the plant or plant part relative to a control plant or plant part, e.g., when grown under the same environmental condition, as further described in the present disclosure.

### (Ii) Plants With Reduced Beta-Amyrin Synthase Activity

The plants, plant parts (e.g., seeds, fruit), or plant products (e.g., plant protein composition) of the present disclosure can comprise reduced activity of beta-amyrin synthase compared to a control plant, plant part, or plant product. Also provided herein is a population of plants or plant parts (e.g., seeds) comprising the plants and plant parts of the present disclosure, which has reduced BAS activity compared to a control (e.g., wild-type) population of plants or plant parts.

In particular, the BAS activity in the plant, plant part, population of plants or plant parts, or plant product of the present disclosure can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, population, or plant product. Activity of beta-amyrin synthase can be measured by one or more standard methods of measuring enzyme activity, e.g., enzyme assays. For example, BAS activity in a plant, plant part, or plant product can be determined by contacting a substrate (e.g., 2, 3-oxidosqualene) with a sample obtained from a plant, plant part, or plant product and measuring the level of the product, e.g., beta-amyrin, e.g., by gas chromatography-mass spectrometry (GC-MS).

Further, levels of beta-amyrin in the plant, plant part, or plant product of the present disclosure can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, 100%, or at least 10%, 15%, 20%, 25%, 30%, 35%, 40%, 45%, 50%, 55%, 60%, 65%, 70%, 75%, 80%, 85%, 90%, 95%, 100% as compared to a control plant or plant part. Levels of beta-amyrin can be measured by standard methods of measuring triterpenoid levels, e.g., by GC-MS.

### (Iii) Plants With Reduced Expression Level of BAS Gene or BAS Protein

The plant, plant part (e.g., seeds, fruit), or plant product (e.g., plant protein composition) of the present disclosure, e.g., comprising one or more insertions, substitutions, or deletions in at least one native BAS gene or homolog or in a regulatory region of such BAS gene or homolog, can have reduced expression level of the BAS gene or homolog as compared to the expression level of the BAS gene or homolog in a control plant, plant part, or plant product, e.g., a plant, plant part, or plant product without such mutation. Also provided herein is a population of plants or plant parts (e.g., seeds) comprising the plants and plant parts of the present disclosure, having reduced expression level of BAS gene(s) or BAS protein compared to a control (e.g., wild-type) population.

In particular, the expression levels of BAS gene or homolog in the plant, plant part, population of plants or plant parts, or plant product (e.g., plant protein composition) of the present disclosure can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100%, as compared to the expression level of the BAS gene or homolog in a control plant, plant part, population, or plant product. In specific embodiments, the BAS gene or homolog is a BAS1 gene, e.g., a Glycine max BAS1 gene. Expression levels of the BAS gene or homolog can be measured by any standard methods for measuring mRNA levels of a gene, including quantitative RT-PCR, northern blot, and serial analysis of gene expression (SAGE). Expression levels of the BAS gene or homolog in a plant, plant part, or plant product can also be measured by any standard methods for measuring protein levels, including western blot analysis, ELISA, or dot blot analysis of a protein sample obtained from a plant, plant part, or plant product using an antibody directed to the BAS protein encoded by the BAS gene.

The plant, plant part (e.g., seeds, fruit), population of plants or plant parts, or plant product (e.g., plant protein composition) of the present disclosure, e.g., comprising one or more insertions, substitutions, or deletions in at least one native BAS gene or homolog or in a regulatory region of such BAS gene or homolog can have reduced expression of the BAS protein, e.g., the BAS protein encoded by the BAS gene or homolog (having the mutation in the gene or in its regulatory region), as compared to the expression level of the BAS protein in a control plant, plant part, population, or plant product, e.g., a plant, plant part, or plant product without such mutation. In particular, the expression levels of a full length BAS protein in the plant, plant part, population of plants or plant parts, or plant product of the present disclosure can be reduced as compared to a control plant, plant part, population, or plant product. A “full-length” BAS protein, as used herein, refers to a BAS protein comprising the complete amino acid sequence of a wild-type BAS protein, e.g., encoded by a native BAS gene. A plant, plant part, population of plants or plant parts, or plant product that contains a mutated BAS gene can have reduced expression of full-length BAS protein as compared to a control plant, plant part, population, or plant product, e.g., a plant, plant part, or plant product without such mutation, e.g., a plant, plant part, population, or plant product comprising a native (e.g., wild-type) BAS gene. In some embodiments, in the plant, plant part, population of plants or plant parts, or plant product of the present disclosure, e.g., comprising one or more insertions, substitutions, or deletions in at least one native BAS gene or homolog or in a regulatory region of such BAS gene or homolog, expression of BAS protein, e.g., full length BAS protein, e.g., encoded by the BAS gene is reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to expression of BAS protein, e.g., full length BAS protein in a control plant, plant part, population, or plant product. In specific embodiments, the BAS protein is a BAS1 protein encoded by the BAS1 gene, e.g., Glycine max BAS1 gene. Expression of a BAS protein, such as a full length BAS protein, in a plant, plant part, population of plants or plant parts, or plant product can be determined by one or more standard methods of determining protein levels. For example, expression of a BAS protein can be determined by western blot analysis, ELISA, or dot blot analysis of a protein sample obtained from a plant, plant part, population or plants or plant parts, or plant product using an antibody directed to the BAS protein, e.g., the full-length BAS protein.

### (Iv) Plants With Loss-of-Function or Reduced Function of BAS Protein

The plant, plant part (e.g., seeds, fruit), or plant product (e.g., plant protein composition) of the present disclosure, e.g., comprising one or more insertions, substitutions, or deletions in at least one native BAS gene or homolog or in a regulatory region of such BAS gene or homolog can have loss-of-function or reduced function in the BAS protein, e.g., loss of BAS activity or reduced BAS activity, as compared to the BAS protein in a control plant, plant part, or plant product. Also provided herein is a population of plants or plant parts (e.g., seeds) comprising the plants and plant parts of the present disclosure, which has loss-of-function or reduced function of the BAS protein compared to a control (e.g., wild-type) population of plants or plant parts. A control plant, plant part, population, or plant product can be a plant, plant part, population, or plant product without the mutation, or a plant, plant part, or plant product having wild-type BAS activity. The BAS protein with loss-of-function or reduced function can comprise a mutation compared to a wild-type BAS protein that causes loss or reduction of BAS function. In some embodiments, the function of the BAS protein encoded by the BAS gene or homolog having a mutation (e.g., one or more insertions, substitutions, or deletions) in the gene or its regulatory region is reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to a control BAS protein encoded by a control BAS gene or homolog without such mutation. In some embodiments, the activity of the BAS protein in the plant, plant part, population of plants or plant parts, or plant product having a mutation (e.g., one or more insertions, substitutions, or deletions) in the BAS gene or homolog, or its regulatory region is reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to a control plant, plant part, population, or plant product, e.g., a plant, plant part, or plant product without such mutation. In specific embodiments, the BAS protein is a BAS 1 protein encoded by the BAS1 gene, e.g., Glycine max BAS1 gene. Function or activity of a BAS protein in a plant, plant part, population of plants or plant parts, or plant product can be determined by one or more standard methods for measuring enzyme activity, e.g., enzyme assays. For example, BAS activity in a plant, plant part, population of plants or plant parts, or plant product can be determined by contacting a substrate (e.g., 2, 3-oxidosqualene) with a sample obtained from the plant, plant part, or plant product and measuring the level of the product, e.g., beta-amyrin, e.g., by gas chromatography-mass spectrometry (GC-MS).

### B. Plants With Decreased Saponin Content

The plant, plant part (e.g., seeds, fruit), or plant product (e.g., plant protein composition) of the present disclosure, e.g., comprising a mutation that decreases BAS activity, e.g., comprising one or more insertions, substitutions, or deletions in at least one native BAS gene or homolog or in a regulatory region of such BAS gene or homolog, can have reduced levels of saponins as compared to a control plant, plant part, or plant product, e.g., without such mutation. Also provided herein is a population of plants or plant parts (e.g., seeds) comprising the plants and plant parts of the present disclosure, having decreased saponin content as compared to a control population.

A control plant, plant part, population, or plant product can be a plant or plant part to which a mutation provided herein has not been introduced, e.g., by methods of the present disclosure. Thus, a control plant, plant part, population, or plant product may express a native (e.g., wild-type) BAS gene endogenously or transgenically, and/or may have a wild-type BAS activity. A control plant, plant part, or population of the present disclosure may be grown under the same environmental conditions (e.g., same or similar temperature, humidity, air quality, soil quality, water quality, and/or pH conditions) as a plant, plant part, or population of plants or plant parts of the present disclosure. A plant, plant part, population of plants or plant parts, or plant product of the present disclosure may have decreased saponin content as compared to a control plant, plant part, or plant product, when the plant or plant part of the present disclosure is grown under the same environmental conditions as the control plant or plant part.

In some embodiments, saponin content (e.g., total saponin content, DDMP saponin content) in the plant, plant part, population of plants or plant parts, and/or plant product (e.g., plant protein composition) described herein is reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to a control plant, plant part, population, or plant product. In specific embodiments, saponin content in the plant, plant part, population of plants or plant parts, and/or plant product provided herein are reduced by about 75-100%, at least about 75%, or at least about 97%, as compared to a control plant, plant part, population, or plant product. A control plant or plant part can be a plant or plant part of the same variety without the mutation provided herein, the plant or plant part before the mutation is introduced, a reference plant or plant part, or a commonly available variety of plant or plant part. One skilled in the art can select an appropriate control. In certain cases, seeds of a reference variety of soybean cultivar may contain about 2.7-7.0 mg/g of total saponins, of which about 60-80% can be DDMP saponins (which is one of the most astringent species of saponins). The seeds of the plant, plant part, population of plants or plant parts, or a population of seeds provided herein can contain from about 0 mg/g to about 0.8 mg/g of total saponins, and/or from about 0 mg/g to about 0.6 mg/g of DDMP saponins. Saponin content in a plant, plant part, or plant product can be measured by any standard methods of measuring or estimating the amount of saponins in a sample. For example, saponin content can be determined by using colorimetry, high performance liquid chromatography (HPLC), mass spectrometry, or liquid chromatography and tandem mass spectrometry (e.g., LC-MS/MS).

### C. Plants and Plant Products With Improved Flavor Characteristics

The plant, plant part (e.g., seeds, fruit), population of plants or plant parts, or plant product (e.g., plant protein composition) of the present disclosure, e.g., comprising a mutation that decreases BAS activity, e.g., comprising one or more insertions, substitutions, or deletions in at least one native BAS gene or homolog or in a regulatory region of such BAS gene or homolog, can have improved flavor characteristics which may result from reduced saponin content, as compared to a control plant, plant part, population, or plant product, e.g., without the mutation.

“Flavor characteristics” of plant, plant part, population of plants or plant parts, or plant product may refer to taste or aroma of the plant, plant part, or plant product. Aroma can relate to the ratios and intensities of volatile compounds, organic compounds, or protein compounds in the plant, plant part, population, or plant product. Saponin content that contributes to flavor characteristics of a plant, plant part, population of plants or plant parts, or plant product can be quantified by using colorimetry, high performance liquid chromatography (HPLC), mass spectrometry, or liquid chromatography and tandem mass spectrometry (e.g., LC-MS/MS). In specific embodiments, saponin level can contribute a bitterness or off-flavor characteristic that is reduced by decreasing BAS activity in the plant, plant part, population of plants or plant parts, or plant product. Volatile compounds that contribute to flavor characteristics of a plant, plant part, or plant product can be quantified by using gas chromatography - mass spectroscopy (GC-MS) that separates and identifies compounds in their gaseous forms based on their masses. In certain instances, to correlate these instrumental measurements to consumer perception, two major methods of sensory evaluation are used: consumer testing and descriptive analysis. Consumer testing includes subjective data about the preferences of a large group of untrained tasters (usually more than 100 panelists), while descriptive analysis includes questionnaires for a panel of 8-12 trained tasters who are able to rate specific attributes related to flavor or aroma. Methods for determining flavor characteristic of plants and plant parts is described in the art, e.g., by Barrett et al. (Critical Reviews in Food Science and Nutrition, 50(5): 369-389 (2010)) and Hallowell et al. (Chem Senses, 41(3):249-259 (2016)). In certain instances, the methods provided herein can improve flavor characteristics of plant, plant part, population of plants or plant parts, or plant product (e.g., plant protein composition) by a flavor panel experiment. Such flavor panel experiment may use instrumental measurements, sensory testing, or a combination thereof. Plant, plant part, population of plants or plant parts, or plant product (e.g., plant protein composition) that scores higher (as compared to a suitable control) in such flavor panel experiments can be considered to have improved flavor characteristics. For example, in a flavor panel experiment, a plant, plant part, population, or plant product (e.g., plant protein composition) of the present disclosure, e.g., comprising a mutation that decreases BAS activity, e.g., comprising one or more insertions, substitutions, or deletions in at least one native BAS gene or homolog or in a regulatory region of such BAS gene or homolog, can score higher compared to a control plant, plant part, population, or plant product (e.g., not containing the mutation), and thus can be considered to have improved flavor characteristics compared to the control plant, plant part, population, or plant product (e.g., plant protein composition). In specific embodiments, the improved flavor characteristic is reduced bitterness and/or off-flavors. Thus a plant, plant part, or plant product having reduced BAS activity can have reduced bitterness and/or off flavors when compared to a control plant.

### (I) Plant Parts and Plant Products

The present disclosure provides plant parts and plant products obtained from the plant of the present disclosure. A “plant part”, as used herein, refers to any part of a plant, including plant cells, embryos, pollen, ovules, seeds, leaves, flowers, branches, fruit, kernels, ears, cobs, husks, stalks, roots, root tips, anthers, plant protoplasts, plant cell tissue cultures from which plants can be regenerated, plant calli, plant clumps, juice, pulp, nectar, stems, branches, and bark. A “plant product”, as used herein, refers to any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein. Plant parts and plant products provided herein can be intended for human or animal consumption.

As used herein, a “protein product” or “protein composition” refers to any protein composition or product isolated, extracted, and/or produced from plants or plant parts (e.g., seed) and includes isolates, concentrates, and flours, e.g., soy protein composition, soy protein concentrate (SPC), soy protein isolate (SPI), soy flour, flake, white flake, texturized vegetable protein (TVP), or textured soy protein (TSP)). A protein composition can be a concentrated protein solution (e.g., soybean protein concentrate solution) in which the protein is in a higher concentration than the protein in the plant from which the protein composition is derived. The protein composition can comprise multiple proteins as a result of the extraction or isolation process. In specific embodiments, the protein composition can further comprise stabilizers, excipients, drying agents, desiccating agents, anti-caking agents, or any other ingredient to make the protein fit for the intended purpose. The protein composition can be a solid, liquid, gel, or aerosol and can be formulated as a powder. The protein composition can be extracted in a powder form from a plant and can be processed and produced in different ways, such as: (i) as an isolate - through the process of wet fractionation, which has the highest protein concentration; (ii) as a concentrate - through the process of dry fractionation, which are lower in protein concentration; and/or (iii) in textured form -when it is used in food products as a substitute for other products, such as meat substitution (e.g. a “meat” patty). Protein isolate can be derived from defatted soy flour with a high solubility in water, as measured by the nitrogen solubility index (NSI). The aqueous extraction is carried out at a pH below 9. The extract is clarified to remove the insoluble material and the supernatant liquid is acidified to a pH range of 4-5. The precipitated protein-curd is collected and separated from the whey by centrifuge. The curd can be neutralized with alkali to form the sodium proteinate salt before drying. Protein concentrate can be produced by immobilizing the soy globulin proteins while allowing the soluble carbohydrates, whey proteins, and salts to be leached from the defatted flakes or flour. The protein is retained by one or more of several treatments: leaching with 20-80% aqueous alcohol/solvent, leaching with aqueous acids in the isoelectric zone of minimum protein solubility, pH 4-5; leaching with chilled water (which may involve calcium or magnesium cations), and leaching with hot water of heat-treated defatted protein meal/flour (e.g., soy meal/flour). Any of the process provided herein can result in a product that is 70% protein, 20% carbohydrates (2.7 to 5% crude fiber), 6% ash and about 1% oil, but the solubility may differ. As an example, one ton (t) of defatted soybean flakes can yield about 750 kg of soybean protein concentrate. “Texturized vegetable protein” (TVP), “Textured vegetable protein”, also referred to as “textured soy protein” (TSP), soy meat, or soya chunks refers to a defatted plant (e.g., soy) flour product, a by-product of extracting plant (e.g., soybean) oil. It can be used as a meat analogue or meat extender. It is quick to cook, with a protein content comparable to certain meats. TVP can be produced from any proteinrich seed meal left over from vegetable oil production. A wide range of pulse seeds other than soybean, such as lentils, peas, and fava beans, or peanut may be used for TVP production. TVP can be made from high protein (e.g., 50%) soy isolate, flour, or concentrate, and can also be made from cottonseed, wheat, and oats. It is extruded into various shapes (chunks, flakes, nuggets, grains, and strips) and sizes, exiting the nozzle while still hot and expanding as it does so. The defatted thermoplastic proteins are heated to 150-200° C., which denatures them into a fibrous, insoluble, porous network that can soak up as much as three times its weight in liquids. As the pressurized molten protein mixture exits the extruder, the sudden drop in pressure causes rapid expansion into a puffy solid that is then dried. As much as 50% protein when dry, TVP can be rehydrated at a 2:1 ratio, which drops the percentage of protein to an approximation of ground meat at 16%. TVP can be used as a meat substitute. When cooked together, TVP can help retain more nutrients from the meat by absorbing juices normally lost. Also provided herein are methods of isolating, extracting, or preparing any of the protein compositions or protein products provided herein from plants or plant parts.

In specific embodiments, the plant protein compositions provided herein are obtained from a soybean plant (Glycine max) that contains a mutation that decreases BAS activity, e.g., one or more insertions, substitutions, or deletions in at least one native BAS gene or homolog or in a regulatory region of such BAS gene or homolog.

Also provided herein are food and/or beverage products containing plant compositions (e.g., plant parts, plant extract, plant concentrate, plant powder, plant protein, and plant biomass) described herein, such as plant compositions derived from the plants or plant parts of the present disclosure. Food and/or beverage products of the present disclosure can include shakes (e.g., protein shakes), health drinks, alternative meat products (e.g., meatless burger patties, meatless sausages), alternative egg products (e.g., eggless mayo), non-dairy products (e.g., non-dairy whipped toppings, non-dairy milk, non-dairy creamer, non-dairy milk shakes, non-diary ice cream), energy bars (e.g., protein energy bars), infant formula, baby foods, cereals, baked goods, edamame, tofu, and tempeh. A food and/or beverage product that contains plant compositions obtained from plants or plant parts of the present disclosure can have desired traits, compared to a similar or comparable food and/or beverage product that contains plant compositions obtained from a control plant or plant part.

Plant parts (e.g., seeds) and plant products (e.g., plant biomass, seed compositions, protein compositions, food and/or beverage products) produced from the plant or plant part provided herein can be meant for consumption by agricultural animals or for use as feed in an agriculture or aquaculture system. In specific embodiments, plant parts and plant products produced from the plant or plant part provided herein include animal feed (e.g., roughages - forage, hay, silage; concentrates - cereal grains, soybean cake) intended for consumption by bovine, porcine, poultry, lambs, goats, or any other agricultural animal. In specific embodiments, plant parts and plant products include aquaculture feed for any type of fish or aquatic animal in a farmed or wild environment including, without limitation, trout, carp, catfish, salmon, tilapia, crab, lobster, shrimp, oysters, clams, mussels, and scallops.

Seeds of the present disclosure include a representative sample of seeds, from a plant of the present disclosure. A plant or plant part of the present disclosure can be a crop plant or part of a crop plant.

As provided herein, the plant parts, population of plant parts, and plant products, including plant protein compositions and plant-based food/beverage products of the present disclosure can contain a mutation that decreases BAS activity, e.g., one or more insertions, substitutions, or deletions in at least one native BAS gene or homolog or in a regulatory region of such BAS gene or homolog, e.g., a deletion of about 4-78 nucleotides at least partially in the nucleic acid region of exon 7 of the Glycine max BAS1 gene, a substitution in the nucleic acid region of exon 2 of the Glycine max BAS1 gene, and/or a substitution in the nucleic acid region of exon 4 of the Glycine max BAS1 gene. The plant parts, population of plant parts, and plant products of the present disclosure can have reduced BAS activity, reduced expression level of the BAS gene or homolog, reduced expression level of the BAS protein (e.g., the full-length BAS protein) encoded by the BAS gene, loss of function or reduced function or activity of the BAS protein encoded by the BAS gene, reduced saponin levels, and/or improved flavor characteristics compared to a control plant part, population, or plant product, e.g., without the mutation, comprising a native (e.g., wild-type) BAS gene or BAS protein, or comprising wild-type BAS activity.

### IV. Altering Saponin Content in Plants

Methods are provided herein for altering (e.g., decreasing) saponin content in a plant or plant part. In some aspects, the methods comprise reducing beta-amyrin synthase (BAS) activity in the plant or plant part, by, e.g., reducing levels or activity of a BAS protein. Levels or activity of beta-amyrin synthase in a plant or plant part can be reduced by any methods known in the art for reducing protein activity or reducing gene expression, including the methods provided herein.

In some aspects, the methods comprise introducing a genetic mutation that alters (e.g., decreases) beta-amyrin synthase (BAS) activity into a plant or plant part. The method can further comprise introducing the genetic mutation that alters (e.g., decreases) BAS activity into a plant cell, and regenerating a plant or plant part from the plant cell (e.g., transformed plant cell). The methods provided herein can alter (e.g., decrease) beta-amyrin synthase (BAS) level or activity, alter (e.g., decrease) expression levels of at least one BAS gene encoding BAS protein, alter (e.g., decrease) BAS protein levels or activity, alter (e.g., decrease) beta-amyrin levels, alter (e.g., decrease) saponin content, and/or alter (e.g., improve) flavor characteristics in the plant or plant part compared to a control plant or plant part. A control plant or plant part can be a plant or plant part to which a mutation provided herein has not been introduced, e.g., by methods of the present disclosure. Thus, a control plant or plant part (e.g., seeds, fruit) may express a native (e.g., wild-type) BAS gene endogenously or transgenically. A control plant of the present disclosure may be grown under the same environmental conditions (e.g., same or similar temperature, humidity, air quality, soil quality, water quality, and/or pH conditions) as a plant to which the mutation is introduced according to the methods provided herein. A plant, plant part (e.g., seeds, fruit), population of plants or plant parts, or plant product (e.g., plant protein compositions) produced according to the methods of the present disclosure may have the mutation that decreases BAS activity, altered (e.g., decreased) expression levels of at least one BAS gene or homolog thereof, altered (e.g., decreased) BAS protein levels or activity, altered (e.g., decreased) beta-amyrin levels, altered (e.g., decreased) saponin content, and/or altered (e.g., improved) flavor characteristics as compared to a control plant, plant part, or population of plants or plant parts, when the plant, plant part, or the population of the present disclosure is grown under the same environmental conditions as the control plant, plant part, or population.

### A. Altering Expression or Function of Beta-Amyrin Synthase in Plants

Provided herein are compositions and methods for altering (e.g., decreasing) saponin content in a plant or plant part by introducing a genetic mutation that alters (e.g., decreases) beta-amyrin synthase (BAS) activity into a plant or plant part. The method can further comprise introducing the genetic mutation that alters (e.g., decreases) BAS activity into a plant cell, and regenerating a plant or plant part from the plant cell (e.g., transformed plant cell). The genetic mutation that is introduced into the plant or plant part according to the methods provided herein can comprise one or more insertions, substitutions, or deletions into the genome of the plant or plant part. The genetic mutation that alters (e.g., decreases) the BAS activity can be introduced into at least one native BAS gene or homolog thereof; a regulatory region of the native BAS gene or homolog thereof; in a coding region, a non-coding region, or a regulatory region of any other gene; or at any other site in the genome of the plant or plant part. A “native” gene refers to any gene having a wild-type nucleic acid sequence, e.g., a nucleic acid sequence that can be found in the genome of a plant existing in nature, including a gene that does not naturally occur within the plant, plant part, or plant cell comprising the gene. For example, a transgenic BAS gene located at a genomic site or in a plant in a non-naturally occurring matter is a “native” BAS gene if its nucleic acid sequence can be found in a plant existing in nature. A “regulatory region” of a gene can include a genomic site where a RNA polymerase, a transcription factor, or other transcription modulators bind and interact to control mRNA synthesis of the gene, such as a promoter region, a binding site for transcription modulator proteins (e.g., transcription factors), and other genomic regions that contribute to regulation of transcription of the gene. A regulatory region of the gene can be located in the 5′ untranslated region of the gene.

In some aspects, the methods of the present disclosure comprise introducing a genetic mutation that decreases the BAS activity into a plant or plant part. The genetic mutation that is introduced into the plant or plant part can comprise one or more (e.g., about 1, 2, 3, 4, 5, 6, 7, 8, 9, 10, 11, 12, 13, 14, 15, 16, 17, 18, 19, 20, or more) insertions, substitutions, and/or deletions in at least one native BAS gene or homolog thereof and/or in a regulatory region of said at least one native BAS gene or homolog thereof in a genome of said plant or plant part. A plant or plant part to which the mutation is introduced according to the methods provided herein can comprise 1-10, 1-5, 2-9, 2-8, 2-7, 2-6, 2-5, 3-5, 4-5 (e.g., 1, 2, 3, 4, 5, 6, 7, 8, 9, or 10) copies of BAS gene, e.g., BAS1, BAS2, BAS3, BAS4, and BAS5 genes, each encoding a BAS protein. In particular, the plant or plant part to which the mutation is introduced according to the methods can comprise at least 2 genes encoding a BAS protein, such as 2, 3, 4, 5, 6, 7, 8, 9, or 10 genes that have less than 100% (e.g., less than 100%, 99%, 98%, 97%, 96%, 95%, 94%, 93%, 92%, 91%, 90%, 89%, 88%, 87%, 86%, or 85%) sequence identity to one another. The methods can comprise introducing one or more (e.g., about 1, 2, 3, 4, 5, 6, 7, 8, 9, 10, 11, 12, 13, 14, 15, 16, 17, 18, 19, 20, or more) insertions, substitutions, and/or deletions: into one BAS gene or homolog; into a regulatory region of one BAS gene or homolog; into more than one (e.g., 2, 3, 4, 5, 6, 7, 8, 9, 10), but not all BAS genes or homologs; into regulatory regions of more than one (e.g., 2, 3, 4, 5, 6, 7, 8, 9, 10), but not all BAS genes or homologs; into all BAS genes or homologs; and/or into regulatory regions of all BAS genes or homologs in the plant or plant part.

The mutation that decreases the BAS activity can be introduced into one or more (e.g., one, more than one but not all, or all) Glycine max BAS genes, such as a Glycine max BAS1 gene, a Glycine max BAS2 gene, a Glycine max BAS3 gene, a Glycine max BAS4 gene, a Glycine max BAS5 gene and/or a regulatory region of such one or more Glycine max BAS genes. In some embodiments, the mutation is introduced into a Glycine max BAS1 gene and/or a regulatory region of the Glycine max BAS1 gene. In some embodiments, the mutation that decreases the BAS activity can be introduced into a BAS gene or homolog thereof comprising a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of any one of SEQ ID NOs: 1-5 and 38 and encoding a polypeptide that retains BAS activity, for example the nucleic acid sequence of any one of SEQ ID NOs: 1-5 and 38; and/or a regulatory region of the BAS gene or homolog thereof comprising such nucleic acid sequence. Additionally, the mutation can be introduced into a BAS gene or homolog thereof encoding a polypeptide comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of any one of SEQ ID NOs: 6-10 and retaining BAS activity, for example a polypeptide comprising the amino acid sequence of any one of SEQ ID NOs: 6-10; and/or a regulatory region of the BAS gene or homolog thereof encoding such polypeptide. In certain embodiments, the mutation that decreases the BAS activity is introduced into a BAS1 gene or homolog thereof or regulatory region thereof. In specific embodiments, the mutation that decreases the BAS activity is introduced into a BAS1 gene or homolog thereof comprising a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence of SEQ ID NO: 1 or 38 and encoding a polypeptide that retains BAS activity, for example the nucleic acid sequence of SEQ ID NO: 1 or 38, and/or a regulatory region of the BAS1 gene or homolog thereof comprising such nucleic acid sequence. Additionally, the mutation can be introduced into a BAS1 gene or homolog thereof encoding a polypeptide comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to an amino acid sequence of SEQ ID NO: 6 and retaining BAS activity, for example a polypeptide comprising an amino acid sequence of SEQ ID NO: 6; and/or a regulatory region of the BAS1 gene or homolog thereof encoding such polypeptide.

The methods provided herein to introduce a mutation that decreases the BAS activity can include introducing at least one (e.g., about 1, 2, 3, 4, 5, 6, 7, 8, 9, 10, 11, 12, 13, 14, 15, 16, 17, 18, 19, 20, or more) insertion, substitution, or deletion into a nucleic acid region of exon 10 or upstream of exon 10 of a Glycine max BAS1 gene in the plant or plant part. The methods can include introducing at least one (e.g., about 1, 2, 3, 4, 5, 6, 7, 8, 9, 10, 11, 12, 13, 14, 15, 16, 17, 18, 19, 20, or more) insertion, substitution, or deletion at least partially into a nucleic acid region of exon 2, 4, and/or 7 of the Glycine max BAS1 gene in the plant or plant part. For instance, the whole part of the insertion, the substitution, or the deletion can be introduced within exon 7 of the Glycine max BAS1 gene, or can span across the exon and a region (e.g., an intron, a regulatory region) upstream or downstream of the exon. In some embodiments, the methods provided herein include introducing a deletion of about 4-78 nucleotides at least partially in the nucleic acid region of exon 7 of the Glycine max BAS1 gene in the plant or plant part. For example, in specific embodiments, (i) a deletion of nucleotides 4191 through 4195 of SEQ ID NO: 1 (at chr07:137242 to 137246 in the Glycine max BAS1 gene), (ii) a deletion of nucleotides 4190 through 4199 of SEQ ID NO: 1, (iii) a deletion of nucleotides 4171 through 4198 of SEQ ID NO: 1, (iv) a deletion of nucleotides 4187 through 4190 of SEQ ID NO: 1, (v) a deletion of nucleotides 4189 through 4198 of SEQ ID NO: 1, (vi) a deletion of nucleotides 4120 through 4197 of SEQ ID NO: 1, (vii) a deletion of nucleotides 4187 through 4191 of SEQ ID NO: 1, (viii) a deletion of nucleotides 4188 through 4195 of SEQ ID NO: 1, and/or (ix) a deletion of nucleotides 4187 through 4194 of SEQ ID NO: 1is introduced into the plant or plant part.

Additionally or alternatively, the methods provided herein can include introducing a mutation into the Glycine max BAS1 gene that results in a G to E substitution of amino acid 220 of SEQ ID NO: 6 in the BAS protein, such as a G to A substitution of nucleotide 3564 of SEQ ID NO: 1 or nucleotide 3750 of SEQ ID NO: 38 (at chr07:136615 of the Glycine max BAS1 gene), and/or a mutation into the Glycine max BAS1 gene that results in an R to W substitution of amino acid 100 of SEQ ID NO: 6 in the BAS protein, such as an A to T substitution of nucleotide 374 of SEQ ID NO: 1 or nucleotide 560 of SEQ ID NO: 38 (at chr07:133425 of the Glycine max BAS1 gene).

The mutation introduced into the plant or plant part according to the methods of the present disclosure can comprise an out-of-frame mutation of at least one (e.g., one, more than one but not all, or all) BAS gene or homolog thereof. Alternatively, the mutation introduced into the plant or plant part according to the methods can comprise an in-frame mutation, such as a missense mutation, or a nonsense mutation of at least one (e.g., one, more than one but not all, or all) BAS gene or homolog thereof.

### Introducing Regulatory Modifications

The methods described herein can comprise introducing a mutation that decreases the BAS activity, e.g., one or more (e.g., about 1, 2, 3, 4, 5, 6, 7, 8, 9, 10, 11, 12, 13, 14, 15, 16, 17, 18, 19, 20, or more) insertions, substitutions, and/or deletions into a regulatory region of at least one (e.g., one, more than one but not all, or all) BAS gene. For example, one or more insertions, substitutions, and/or deletions can be introduced into a promoter region, a transcription modulator protein (e.g., transcription factor) binding site, or other regulatory regions of at least one (e.g., one, more than one but not all, or all) BAS gene to confer to the plant or plant part an altered (e.g., reduced) transcription activity of the BAS gene.

In some embodiments, the methods provided herein include introducing a mutation into a promoter region of at least one (e.g., one, more than one but not all, or all) BAS gene. The one or more insertions, substitutions, and/or deletions in the promoter region of the BAS gene can alter the transcription initiation activity of the promoter. For example, the modified promoter can reduce transcription of the operably linked nucleic acid molecule (e.g., the BAS gene), initiate transcription in a developmentally-regulated or temporally-regulated manner, initiate transcription in a cell-specific, cell-preferred, tissue-specific, or tissue-preferred manner, or initiate transcription in an inducible manner. A deletion, a substitution, or an insertion, e.g., introduction of a heterologous promoter sequence, a cis-acting factor, a motif or a partial sequence from any promoter, including those described elsewhere in the present disclosure, can be introduced into the promoter region of the BAS gene to confer an altered (e.g., reduced) transcription initiation function according to the present disclosure.

The promoter sequence of one or more BAS genes can be inactivated by insertion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. Additionally or alternatively, the promoter sequence of one or more of BAS genes can be inactivated by deletion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. The promoter sequence of one or more BAS genes can also be inactivated by replacement of the promoter sequence with one or more substitutes. In particular, the substitute can be a cisgenic substitute, a transgenic substitute, or both.

In some instances, the promoter sequence of one or more BAS genes is inactivated by correction of the promoter sequence. A promoter sequence may be corrected by deletion, modification, and/or correction of one or more polymorphisms or mutations that would otherwise enhance the activity of the promoter sequence. In particular, the promoter sequence of one or more BAS genes can be inactivated by: (i) detection of one or more polymorphism or mutation that enhances the activity of the promoter sequence; and (ii) correction of the promoter sequences by deletion, modification, and/or correction of the polymorphism or mutation.

In some instances, the promoter sequence of one or more BAS genes is inactivated by insertion, deletion, and/or modification of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) upstream nucleotide sequences.

In some instances, the promoter sequence of one or more BAS genes is inactivated by addition, insertion, and/or engineering of cis-acting factors that interact with and modify the promoter sequence.

Function and/or expression of the one or more BAS genes can also be decreased or inhibited by modulation (e.g., increase or decrease) of expression of one or more transcription factor genes. For example, modulation of expression of the one or more transcription factor genes can inactivate or inhibit transcription initiation activity of the promoter of the one or more of BAS genes and/or inhibit expression of the one or more BAS genes.

Function and/or expression of the one or more BAS genes can also be decreased by insertion, modification, and/or engineering of transcription factor binding sites or enhancer elements. For example, insertion of new transcription factor binding sites or enhancer elements can decrease function and/or expression of BAS genes. Alternatively, modification and/or engineering of existing transcription factor binding sites or enhancer elements can decrease function and/or expression of BAS genes.

Function and/or expression of the one or more BAS genes can also be decreased or inhibited by insertion of one or more negative regulatory sequences of the gene. For example, to inhibit the expression and/or function of the BAS gene, a part or whole of one or more negative regulatory sequences of the BAS gene can be inserted in the genome of a plant cell or plant part. The negative regulatory sequence of the gene can be in a cis location. Alternatively, the negative regulatory sequence of the gene may be in a trans location. Negative regulatory sequences of the one or more BAS genes can also include upstream open reading frames (uORFs). In some instances, a negative regulatory sequence can be inserted in a region upstream of the BAS gene in order to inhibit the expression and/or function of the gene.

### Introducing a Mutation Into a Homolog, Ortholog, or Variant of a BAS Gene

A genetic mutation that decreases the BAS activity can be introduced into a gene that is a homolog, ortholog, or variant of a BAS gene disclosed herein and expresses a BAS protein with BAS function, or in a regulatory region of such homolog, ortholog, or variant of a BAS gene, according to the methods provided herein. For example, the mutation (e.g., one or more insertions, substitutions, or deletions that decrease the BAS activity) can be introduced into orthologs of BAS genes including, without limitation, red clover BAS (Trifolium pratense, NCBI ID: MG492000.1), barrel medic BAS (Medicago truncatula, NCBI ID: AJ430607.1), chickpea BAS (Cicer arietinum, NCBI ID: XM_027335420.1), narrow-leaved blue lupine BAS (Lupinus angustifolius, NCBI ID: XM_019600620.1), pigeon pea BAS (Cajanus cajan, NCBI ID: XM _020370843.2, XM_020370845.2, XM_020370844.2, XM_029273321.1), peanut BAS (Arachis hypogaea, NCBI ID: XM_025789404.2, XM_025789405.2), cowpea BAS (Vigna unguiculata, NCBI ID: XM_028051921.1), adzuki bean BAS (Vigna angularis, NCBI ID: XM_017552062.1), mung bean BAS (Vigna radiata, NCBI ID: XM_022787203.1, XM_022787202.1, XM_022787208.1, XM_022787209.1, XM_022787206.1, XM_022787207.1, XM_022787210.1, XM _014662050.2, XM_014662049.2, XM_022787200.1, XM_022787199.1, XM_022787201.1, XM _022787205.1, XM_022787204.1, XM_022787211.1), pea BAS (Pisum sativum, AB034802.1), and licorice BAS (Glycyrrhiza glabra, AB037203.1).

Variant sequences (e.g., homologs, orthologs) can be isolated by PCR. In this manner, variant sequences encoding BAS can be identified and used in the methods of the present disclosure. The variant sequences will retain the BAS activity.

In certain instances, mutations introduced into any BAS gene or its regulatory region in a plant, plant part, or plant product (e.g., plant protein composition) according to the methods provided herein can be identified by a diagnostic method described herein. Such diagnostic methods may comprise use of primers for detecting mutation in a BAS gene. For example, forward primer GATAGTCGTTCATTATGTCAATC (SEQ ID NO: 13) and reverse primer CACACAACCAATGGTTATG (SEQ ID NO: 14) can be used for detection of mutation in Glycine max BAS1 gene near binding site of GmBAS1 guide RNA 6 (SEQ ID NO: 12) in exon 7, e.g., a deletion of nucleotides 4191 through 4195 of SEQ ID NO: 1 (chr07:137242..137246). The forward primer GACTATAGAAGATGGAGAGGAAATCACAT (SEQ ID NO: 15) and the reverse primer AAGAGAGGACCTGCAATTTGAGC (SEQ ID NO: 16) can be used for detection mutation in Glycine max BAS1 gene at or near nucleotide 374 of SEQ ID NO: 1 or nucleotide 560 of SEQ ID NO: 38 (chr07:133425). The probes CCGTCAGATGGGG (SEQ ID NO: 17) and GTCAGAAGGGGCG (SEQ ID NO: 18), optionally coupled with a quencher, e.g., minor groove binder (MGB), can be used for detecting an A to T substitution and a wild-type sequence (A), respectively, at nucleotide 374 of SEQ ID NO: 1 or nucleotide 560 of SEQ ID NO: 38 in the GmBAS1 gene (chr07:133425). The forward primer TAGAGCAAGAAAGTGGATTCGAGA (SEQ ID NO: 19) and the reverse primer CACCGAGTATCTACAAGAGCAAGATC (SEQ ID NO: 20) can be used for detection of mutation in Glycine max BAS1 gene at or near nucleotide 3564 of SEQ ID NO: 1 or nucleotide 3750 of SEQ ID NO: 38 (chr07:136615). The probes TCATGGGAAAAAA (SEQ ID NO: 21) and CTTCATGGGGAAAAA (SEQ ID NO: 22), optionally coupled with a quencher, e.g., MGB, can be used for detecting a G to A substitution and a wild-type sequence (G), respectively, at nucleotide 3564 of SEQ ID NO: 1 or nucleotide 3750 of SEQ ID NO: 38 in the GmBAS1 gene (chr07:136615). In some embodiments, the one or more mutations are integrated into the plant genome and the plant or the plant part is stably transformed according to the methods. In other embodiments, the one or more mutations are not integrated into the plant genome and wherein the plant or the plant part is transiently transformed according to the methods.

Introducing one or mutations insertions, substitutions, or deletions into at least one BAS gene or homolog or in a regulatory region of such BAS gene or homolog in the genome of the plant or plant part can reduce the expression levels of the BAS gene or homolog, reduce level or activity of the BAS protein encoded by the BAS gene or homolog, reduce BAS activity in the plant or plant part, reduce saponin content in the plant or plant part, and/or improve flavor characteristics in the plant or plant part relative to a control plant or plant part, e.g., when grown under the same environmental condition, as further described in the present disclosure.

### (I) Reducing Beta-Amyrin Synthase Activity

The methods of the present disclosure can reduce activity of beta-amyrin synthase (BAS) in plants, plant parts (e.g., seeds, fruit), a population of plants or plant parts, or plant products (e.g., plant protein composition) compared to a control plant, plant part, population, or plant product. In particular, methods provided herein can reduce the BAS activity in the plant, plant part, population, or plant product by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, population, or plant product. Activity of beta-amyrin synthase can be measured by one or more standard methods of measuring enzyme activity, e.g., enzyme assays. For example, BAS activity in a plant, plant part, or plant product can be determined by contacting a substrate (e.g., 2, 3-oxidosqualene) with a sample obtained from a plant, plant part, or plant product and measuring the level of the product, e.g., beta-amyrin, e.g., by gas chromatography-mass spectrometry (GC-MS).

Further, the methods provided herein can reduce levels of beta-amyrin in the plant, plant part, population of plants or plant parts, or plant product of the present disclosure by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, 100%, or at least 10%, 15%, 20%, 25%, 30%, 35%, 40%, 45%, 50%, 55%, 60%, 65%, 70%, 75%, 80%, 85%, 90%, 95%, 100% as compared to a control plant, plant part, population, or plant product. Levels of beta-amyrin can be measured by standard methods of measuring triterpenoid levels, e.g., by GC-MS.

### (Ii) Reducing Expression Level of BAS Gene or BAS Protein

The methods of the present disclosure, e.g., introducing one or more insertions, substitutions, or deletions in at least one native BAS gene or homolog or in a regulatory region of such BAS gene or homolog in a plant or plant part can reduce expression level of the BAS gene or homolog in the plant, plant part (e.g., seeds, fruit), population of plants or plant parts, or plant product (e.g., plant protein composition) as compared to the expression level of the BAS gene or homolog in a control plant, plant part, population, or plant product, e.g., a plant, plant part, population, or plant product without such mutation. In particular, the methods provided herein can reduce the expression levels of BAS gene or homolog in the plant, plant part, population of plants or plant parts, or plant product (e.g., plant protein composition) by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100%, as compared to the expression level of the BAS gene or homolog in a control plant, plant part, population, or plant product. In specific embodiments, the methods provided herein can reduce expression levels of a BAS1 gene, e.g., a Glycine max BAS1 gene. Expression levels of the BAS gene or homolog can be measured by any standard methods for measuring mRNA levels of a gene, including quantitative RT-PCR, northern blot, and serial analysis of gene expression (SAGE). Expression levels of the BAS gene or homolog in a plant, plant part, or plant product can also be measured by any standard methods for measuring protein levels, including western blot analysis, ELISA, or dot blot analysis of a protein sample obtained from a plant, plant part, or plant product using an antibody directed to the BAS protein encoded by the BAS gene.

The methods of the present disclosure, e.g., introducing one or more insertions, substitutions, or deletions in at least one native BAS gene or homolog or in a regulatory region of such BAS gene or homolog can reduce expression levels of the BAS protein, e.g., the BAS protein encoded by the BAS gene or homolog (having the mutation in the gene or in its regulatory region) in the plant, plant part (e.g., seeds, fruits), population of plants or plant parts, and plant product (e.g., plant protein compositions), as compared to the expression level of the BAS protein in a control plant, plant part, population, or plant product, e.g., a plant, plant part, population, or plant product without such mutation. In particular, the methods provided herein can reduce the expression levels of a full length BAS protein (e.g., a BAS protein having the complete amino acid sequence of a wild-type BAS protein, e.g., encoded by a native BAS gene) in the plant, plant part, population of plants or plant parts, or plant product (e.g., plant protein composition) as compared to a control plant, plant part, population, or plant product. The methods provided herein can introduce a mutation into at least one BAS gene or its regulatory regions in the plant or plant part, which can reduce expression of full-length BAS protein in the plant, plant part, population of plants or plant parts, or plant product (e.g., plant protein composition) as compared to a control plant, plant part, population, or plant product, e.g., product without such mutation, e.g., comprising a native (e.g., wild-type) BAS gene. In particular, the methods provided herein, e.g., introducing one or more insertions, substitutions, or deletions in at least one native BAS gene or homolog or in a regulatory region of such BAS gene or homolog, can reduce expression levels of BAS protein, e.g., full length BAS protein, e.g., encoded by the BAS gene by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to expression of BAS protein, e.g., full length BAS protein in a control plant, plant part, or plant product. In specific embodiments, the methods provided herein can reduce expression levels of a BAS1 protein encoded by the BAS1 gene, e.g., Glycine max BAS1 gene. Expression of a BAS protein, such as a full length BAS protein, in a plant, plant part, population of plants or plant parts, or plant product can be determined by one or more standard methods of determining protein levels. For example, expression of a BAS protein can be determined by western blot analysis, ELISA, or dot blot analysis of a protein sample obtained from a plant, plant part, or plant product using an antibody directed to the BAS protein, e.g., the full-length BAS protein.

### (Iii) Reducing or Eliminating Function of BAS Protein

The methods of the present disclosure, e.g., introducing one or more insertions, substitutions, or deletions in at least one native BAS gene or homolog or in a regulatory region of such BAS gene or homolog can reduce or remove (e.g., reduce to zero) function in the BAS protein, e.g., reduce or remove BAS activity, as compared to the BAS protein in a control plant, plant part, population, or plant product. A control plant, plant part, population, or plant product can be a plant, plant part, population, or plant product without the mutation, or a plant, plant part, population, or plant product having wild-type BAS activity. The methods disclosed herein can produce a BAS protein with loss-of-function or reduced function having a mutation compared to a wild-type BAS protein that causes loss or reduction of BAS function. In some embodiments, the methods provided herein can reduce the function of the BAS protein encoded by the BAS gene or homolog to which a mutation (e.g., one or more insertions, substitutions, or deletions) has been introduced in the gene or its regulatory region by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to a control BAS protein encoded by a control BAS gene or homolog without such mutation. In some embodiments, the methods provided herein can reduce the activity of the BAS protein in the plant, plant part, population of plants or plant parts, or plant product to which the mutation (e.g., one or more insertions, substitutions, or deletions) has been introduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to a control plant, plant part, population, or plant product, e.g., a plant, plant part, population, or plant product without such mutation. In specific embodiments, the methods can reduce or remove activity or function of a BAS1 protein encoded by the BAS1 gene, e.g., Glycine max BAS1 gene. Function or activity of a BAS protein in a plant, plant part, population of plants or plant parts, or plant product can be determined by one or more standard methods for measuring enzyme activity, e.g., enzyme assays. For example, BAS activity in a plant, plant part, population of plants or plant parts, or plant product can be determined by contacting a substrate (e.g., 2, 3-oxidosqualene) with a sample obtained from the plant, plant part, population of plants or plant parts, or plant product and measuring the level of the product, e.g., beta-amyrin, e.g., by gas chromatography-mass spectrometry (GC-MS).

### B. Introducing a Mutation into the Genome of Plant Cells

Introducing one or more mutations into the plant genome, e.g., into at least one BAS gene (e.g., Glycine max BAS1) or its regulatory region, and modulating the level or activity of BAS in a plant or plant part may be achieved in any method of creating a change in a nucleic acid of a plant. For example, one or more mutations can be introduced into the plant genome, e.g., into at least one BAS gene (e.g., Glycine max BAS1) or its regulatory region by contacting the plant or plant part with a mutagen. A “mutagen” as used herein refers to an agent (e.g., a physical or chemical agent) that, upon exposure to an organism or a genetic material (e.g., DNA), introduces a mutation into the genetic material. Physical mutagens that can be used in the methods provided herein include electromagnetic radiation, such as gamma rays, X rays, and UV light, and particle radiation, such as fast and thermal neutrons, beta and alpha particles. Chemical mutagens react with DNA and lead to faulty base pairing. Chemical mutagens that can be used in the methods provided herein include alkylating agents. Alkylating agents include ethyl methanesulfonate (EMS), N-ethyl-N-nitrosourea (ENU), nitrogen mustards, mitomycin, methyl methane sulfonate (MMS), diethyl sulfate, and nitrosoguanidine. EMS produces random mutations in genetic material by nucleotide substitution, particularly through G:C to A:T transitions induced by guanine alkylation, typically producing point mutations. ENU produces mutations by transferring the ethyl group of ENU to nucleobases (usually thymine) in nucleic acids. Chemical mutagens that can be used in the methods provided herein also include DNA intercalating agents, such as acridine orange, ethidium bromide (EtBr), proflavin, and daunorubicin. Chemical mutagens that can be used in the methods provided herein further include base analogues, such as halouracils and uridine derivatives, e.g., 5-bromodeoxyuridine (BrdU), which mimic a particular nucleobase in nucleic acid and are misread by the replicating machinery as a normal base. Following incorporation into DNA, they form non-Watson pairing with the DNA. BrdU is capable of inducing point mutations by substituting thymine residues and pairing with guanine instead of adenine. Chemical mutagens that can be used in the methods provided herein also include nitrous acid, hydroxyl amine, and sodium azide, which can modify the bases by deamination, thus modifying the regular base pairing. Nitrous acid deaminates adenine, guanine, and cytosine substituting adenine to hypoxanthine, guanine to xanthine, and cytosine to uracil. These substitutions induce AT to GC transitions leading to faulty base pairing. In specific embodiments, the methods provided herein includes introducing a mutation that reduces BAS activity into a plant or plant part by contacting the plant or plant part with EMS and/or ENU. In specific embodiments, the methods include contacting the plant or plant part concurrently with EMS and ENU to introduce a mutation.

Additionally or alternatively, one or more mutations can be introduced into the plant genome, e.g., into at least one BAS gene (e.g., Glycine max BAS1) or its regulatory region through the use of precise genome-editing technologies to modulate the expression of the endogenous or transgenic sequence. In this manner, a nucleic acid sequence can be inserted, substituted, or deleted proximal to or within a native plant sequence corresponding to at least one BAS gene through the use of methods available in the art. Such methods include, but are not limited to, use of a nuclease designed against the plant target genomic sequence of interest (D′Halluin et al 2013 Plant Biotechnol J 11: 933-941), such as the Type II CRISPR system, the Type V CRISPR system, the CRISPR-Cas9 system, the CRISPR-Cas12a (Cpf1) system, the transcription activator-like effector nuclease (TALEN) system, the zinc finger nuclease (ZFN) system, and other technologies for precise editing of genomes [Feng et al. 2013 Cell Research 23:1229-1232, Podevin et al. 2013 Trends Biotechnology 31: 375-383, Wei et al. 2013 J Gen Genomics 40:281-289, Zhang et al (2013) WO 2013/026740, Zetsche et al. 2015 Cell 163:759-771]; Natronobacterium gregoryi Argonaute-mediated DNA insertion (Gao et al. 2016 Nat Biotechnol doi: 10.1038/nbt.3547); Cre-lox site-specific recombination (Dale et al. 1995 Plant J 7:649-659; Lyznik, et al. 2007 Transgenic Plant J 1:1-9; FLP-FRT recombination (Li et al. 2009 Plant Physiol 151:1087-1095); Bxb1-mediated integration (Yau et al. 2011 Plant J 701:147-166); zinc-finger mediated integration (Wright et al. 2005 Plant J 44:693-705); Cai et al. 2009 Plant Mol Biol 69:699-709); and homologous recombination (Lieberman-Lazarovich and Levy 2011 Methods MolBiol 701: 51-65; Puchta 2002 Plant Mol Biol 48:173-182). Reagents, components, or compositions that can be used for introducing one or more mutations into plants or plant parts according to the methods of the present disclosure are further described below.

### 1. Editing Reagent

Inserting, substituting, or deleting one or more nucleotides at a precise location of interest in at least one BAS gene and/or a regulatory region of the BAS gene in a plant or plant part may be achieved by introducing into the plant or plant part a system (e.g., a gene editing system), reagents (e.g., editing reagents), or a construct for introducing mutations at the target site of interest in a genome of a plant cell. A “gene editing system”, “editing system”, “gene editing reagent”, and “editing reagent” as used herein, refer to a set of one or more molecules or a construct comprising or encoding the one or more molecules for introducing one or more mutations in the genome. An exemplary gene editing system or editing reagents comprise a nuclease and/or a guide RNA. Also disclosed herein is a construct (e.g., a DNA construct, a recombinant DNA construct) for introducing one or more mutations in plants or plant parts. A construct can comprise an editing system or polynucleotides encoding editing reagents (e.g., nuclease, guide RNA, base editor) each operably linked to a promoter.

As used herein, the terms “nuclease” or “endonuclease” refers to naturally-occurring or engineered enzymes, which cleave a phosphodiester bond within a polynucleotide chain. Nucleases that can be used in precise genome-editing technologies to modulate the expression of the native sequence (e.g., at least one BAS gene and/or a regulatory region of the BAS gene) include, but are not limited to, meganucleases designed against the plant genomic sequence of interest (D′Halluin et al (2013) Plant Biotechnol J 11: 933-941); Cas9 endonuclease; Cas12a (Cpf1) endonuclease; ortholog of Cas 12a endonuclease; Cms1 endonuclease; transcription activator-like effector nucleases (TALENs); zinc finger nucleases (ZFNs); and a deactivated CRISPR nuclease (e.g., a deactivated Cas9, Cas12a, or Cms1 endonuclease) fused to a transcriptional regulatory element (Piatek et al. (2015) Plant Biotechnol J 13:578-589). In some embodiments, the editing system or the editing reagents comprise a zinc finger nuclease (ZFN), a transcription activator-like effector nuclease (TALEN), and/or a clustered regularly interspaced short palindromic repeats (CRISPR) nuclease. In some embodiments, the editing reagents comprise a CRISPR nuclease. In some embodiments, the CRISPR nuclease is a Cas12a nuclease, herein used interchangeably with a Cpf1 nuclease, e.g., a McCpf1 nuclease. In some embodiments, the CRISPR nuclease is a Cas12a nuclease ortholog, e.g., Lb5Cas12a, CMaCas12a, BsCas12a, BoCas12a, MlCas12a, Mb2Cas12a, TsCas12a, and MAD7 endonucleases.

A nuclease system can introduce insertion, substitution, or deletion of genetic elements at a predefined genomic locus by causing a double-strand break at said predefined genomic locus and, optionally, providing an appropriate DNA template for insertion. This strategy is well-understood and has been demonstrated previously to insert a transgene at a predefined location in the cotton genome (D′Halluin et al. 2013 Plant Biotechnol. J. 11: 933-941). For example, a Cas12a (Cpf1) endonuclease coupled with a guide RNA (gRNA) designed against the genomic sequence of interest (i.e., at least one BAS gene and/or a regulatory region of the BAS gene) can be used (i.e., a CRISPR-Cas12a system). Alternatively, a Cas9 endonuclease coupled with a gRNA designed against the genomic sequence of interest (a CRISPR-Cas9 system), or a Cms1 endonuclease coupled with a gRNA designed against the genomic sequence of interest (a CRISPR-Cms1) can be used. Other nuclease systems for use with the methods of the present invention include the CRISPR systems (e.g., Type I, Type II, Type III, Type IV, and/or Type V CRISPR systems (Makarova et al 2020 Nat Rev Microbiol 18:67-83)) with their corresponding gRNA(s), the TALEN system, the ZFN system, the meganuclease system, and the like. Alternatively, a deactivated CRISPR nuclease (e.g., a deactivated Cas9, Cas12a, or Cms1 endonuclease) fused to a transcriptional regulatory element can be targeted to the regulatory region (e.g., upstream regulatory region) of at least one BAS gene, thereby modulating the transcription of the BAS gene (Piatek et al. 2015 Plant Biotechnol J 13:578-589). Site-specific introduction of mutations of plant cells by biolistic introduction of a ribonucleoprotein comprising a nuclease and suitable guide RNA has been demonstrated (Svitashev et al. 2016 Nat Commun doi:10.1038/ncomms13274), and is herein incorporated by reference. For example, a CRISPR system comprises a CRISPR nuclease (e.g., CRISPR-associated (Cas) endonuclease or variant or ortholog thereof, such as Cas12a or Cas12a ortholog) and a guide RNA. A CRISPR nuclease associates with a guide RNA that directs nucleic acid cleavage by the associated endonuclease by hybridizing to a recognition site in a polynucleotide. The guide RNA directs the nuclease to the target site and the endonuclease cleaves DNA at the target site. The guide RNA comprises a direct repeat and a guide sequence, which is complementary to the target recognition site. In certain embodiments, the CRISPR system further comprises a tracrRNA (transactivating CRISPR RNA) that is complementary (fully or partially) to the direct repeat sequence present on the guide RNA. The CRISPR-Cas12a system may comprise at least one guide RNA (gRNA) operatively arranged with the ortholog endonuclease for genomic editing of a target DNA binding the gRNA. The system may comprise a CRISPR-Cas12a expression system encoding the Cas12a ortholog nucleases and crRNAs (CRISPR RNAs) for forming gRNAs that are coactive with the Cas12a nucleases. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

The editing system, editing reagents, or construct described herein can comprise one or more guide RNAs (gRNAs). “Guide RNA” as used herein refers to a RNA molecule that function as guides for RNA- or DNA-targeting enzymes, e.g., nucleases. To introduce one or more mutations into at least one BAS gene and/or the promoter region of the BAS gene, antisense constructions, complementary to at least a portion of the sequence of the BAS messenger RNA (mRNA), BAS gene, or regulatory region of the BAS gene can be constructed. Antisense nucleotides are designed to hybridize with the corresponding mRNA or genomic nucleic acid sequence. Modifications of the antisense sequences may be made as long as the sequences hybridize to and interfere with expression of the corresponding mRNA or genomic sequence. In this manner, antisense constructions having at least 75%, optimally 80%, more optimally 85%, 90%, 95% or greater sequence identity to the corresponding sequences to be edited may be used. Furthermore, portions of the antisense nucleotides may be used to disrupt the expression of the target gene.

Accordingly, a gene editing system, editing reagents, or a construct of the present disclosure can contain a guide RNA (gRNA) cassette to drive mutations at the locus of at least one BAS gene or the regulatory region of the BAS gene. For example, the editing system, the editing reagent, or the construct of the present disclosure may contain a gRNA cassette to drive a deletion (e.g., a 4-78 nucleotide deletion) in a nucleic acid region of exon 10 or upstream of exon 10 of a BAS gene, e.g., a Glycine max BAS1 gene. The gRNA can be specific to a region of exon 10, exon 9, exon 7, exon 5, exon 3, exon 2, or a regulatory region of a BAS gene, e.g., a Glycine max BAS1 gene and/or can drive a deletion at least partially in exon 10, exon 9, exon 7, exon 5, exon 3, exon 2, or a regulatory region of the BAS 1 gene, or active homolog thereof. In certain instances, the gRNA can be specific to exon 7 of a BAS gene, e.g., a Glycine max BAS1 gene. The gRNA can be specific to the nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 11. The gRNA can be specific to the nucleic acid sequence of SEQ ID NO: 11 and/or can drive a deletion (e.g., a 4-78 nucleotide deletion) at least partially in exon 7 or a regulatory region of the Glycine max BAS 1 gene. In particular instances, the gRNA can facilitate binding of an RNA guided nuclease that cleaves a region of at least one BAS gene or a regulatory region of the BAS gene, e.g., Glycine max BAS1 gene and causes non-homologous end joining or homology-directed repair to introduce a mutation at the cleavage site.

In some instances, a gRNA may comprise a targeting region that is complementary to a targeted sequence as well as another region that allows the gRNA to form a complex with a nuclease (e.g., a CRISPR nuclease) of interest. The targeting region (i.e. spacer) of a gRNA that binds to the region of at least one BAS gene or a regulatory region of the BAS gene for use in the method described herein above can be about 100-300 nucleotides long with the targeting region therein about 10-40 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long). For example, the targeting region of a gRNA for use in the method described herein may be 24 nucleotides in length. In specific embodiments, the targeting region of a gRNA is encoded by a nucleic acid sequence comprising a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 12. For example, the targeting region of a gRNA for use in the method described herein can be encoded by a nucleic acid sequence comprising the nucleic acid sequence of SEQ ID NO: 12. The methods provided herein can comprise introducing into the plant, plant part, or plant cell a gRNA comprising a nucleic acid sequence encoded by a nucleic acid sequence that shares at least 80% sequence identity with the nucleic acid sequence of SEQ ID NO: 12 or a nucleic acid sequence of SEQ ID NO: 12, which, along with a nuclease, can introduce a deletion of about 4-78 nucleotides at least partially in the nucleic acid region of exon 7 of the Glycine max BAS1 gene in the plant, plant part, or plant cell. For example, the gRNA can direct a nuclease to a specific target site at exon 7 of the Glycine max BAS1 gene and introduce into the plant, plant part, or plant cell: (i) a deletion of nucleotides 4191 through 4195 of SEQ ID NO: 1, (ii) a deletion of nucleotides 4190 through 4199 of SEQ ID NO: 1, (iii) a deletion of nucleotides 4171 through 4198 of SEQ ID NO: 1, (iv) a deletion of nucleotides 4187 through 4190 of SEQ ID NO: 1, (v) a deletion of nucleotides 4189 through 4198 of SEQ ID NO: 1, (vi) a deletion of nucleotides 4120 through 4197 of SEQ ID NO: 1, (vii) a deletion of nucleotides 4187 through 4191 of SEQ ID NO: 1, (viii) a deletion of nucleotides 4188 through 4195 of SEQ ID NO: 1, or (ix) a deletion of nucleotides 4187 through 4194 of SEQ ID NO: 1. In some embodiments, a gene editing efficiency of the one or more gRNAs is greater than 0.5% (e.g., 0.5%, 1%, 1.5%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100%).

Editing system or editing reagents can also include base editing components. For example, cytosine base editing (CBE) reagents, which change a C-G base pair to a T-A base pair, comprise a single guide RNA, a nuclease (e.g., dCas9, CAS9 nickase), a cytidine deaminase (e.g., APOBEC1), and a uracil DNA glycosylase inhibitor (UGI). Adenine base editing (ABE) reagents, which change an A-T base pair to a G-C base pair comprise a deaminase, (TadA), a nuclease (e.g., dCas or Cas nickase), and a guide RNA.

The gene editing system (e.g., CRISPR-Cas12a system), editing reagents, or a construct of the present disclosure can comprise at least one CRISPR RNA (crRNA) regulatory element operably linked to at least one nucleotide sequence encoding a crRNA for producing gRNA for targeting a target sequence, and at least one regulatory element, which may be the same as or different from the crRNA regulatory element, operably linked to a nucleotide sequence encoding the endonuclease, for generation of a CRISPR editing structure (e.g., CRISPR-Cas12a editing structure) by which the gRNA targets the target sequence and the CRISPR endonuclease cleaves a target DNA to alter gene expression in the cell, and wherein the CRISPR-associated nuclease, and the gRNA, do not naturally occur together. In such system, the at least one crRNA regulatory element may comprise one or more than one RNA polymerase II (Pol II) promoter, or alternatively, a single transcript unit (STU) regulatory element, or one or more of ZmUbi, OsU6, OsU3, and U6 promoters.

The methods described herein, comprising introducing into such plant a non-naturally occurring heterologous CRISPR-Cas12a genomic editing system of a type as variously described herein, can cause the editing reagents to introduce mutations in at least one BAS gene or a regulatory region of the BAS gene and alter the level or activity of BAS gene or BAS protein. The gene editing system (e.g., the CRISPR-Cas12a system) can target PAM sites such as TTN, TTV, TTTV, NTTV, TATV, TATG, TATA, YTTN, GTTA, and/or GTTC.

Such methods of introducing mutations into plants, plant parts, or plant cells may be carried out at moderate temperatures, e.g., below 25° C. and above temperature producing freezing or frost damage of the plant. The methods provided herein may be performed on a wide variety of plants. In particular embodiments, the methods provided herein can be carried out to introduce mutations into the Glycine max plant at one or more BAS genes or a regulatory region of the BAS gene.

Methods disclosed herein are not limited to certain techniques of mutagenesis. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein. Any editing system or editing reagents for use in any genome-editing methods including those described herein can be expressed in a plant or plant part.

### 2. Promoter

As used herein, “promoter” refers to a regulatory region of DNA that is capable of driving expression of a sequence in a plant or plant cell. A number of promoters may be used in the practice of the disclosure, e.g., to express editing reagents in plants, plant parts, or plant cells. The promoter may have a constitutive expression profile. Constitutive promoters include the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like.

Alternatively, promoters for use in the methods of the present disclosure can be tissue-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Alternatively, promoters for use in the methods of the present disclosure can be developmentally-regulated promoters. Such promoters may show a peak in expression at a particular developmental stage. Such promoters have been described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant MolBiol 39: 657-669; Beaudoin and Rothstein (1997) Plant MolBiol 33: 835-846; Genschik et al. (1994) Gene 148: 195-202, and the like.

Alternatively, promoters for use in the methods of the present disclosure can be promoters that are induced following the application of a particular biotic and/or abiotic stress. Such promoters have been described in the art, e.g., Yi et al. (2010) Planta 232: 743-754; Yamaguchi-Shinozaki and Shinozaki (1993) Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

Alternatively, promoters for use in the methods of the present disclosure can be cell-preferred promoters. Such promoters may preferentially drive the expression of a downstream gene in a particular cell type such as a mesophyll or a bundle sheath cell. Such cell-preferred promoters have been described in the art, e.g., Viret et al. (1994) Proc Natl Acad USA 91: 8577-8581; U.S. Pat. No. 8,455,718; U.S. Pat. No. 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like.

It is recognized that a specific, non-constitutive expression profile may provide an improved plant phenotype relative to constitutive expression of a gene or genes of interest. For instance, many plant genes are regulated by light conditions, the application of particular stresses, the circadian cycle, or the stage of a plant’s development. These expression profiles may be important for the function of the gene or gene product in planta. One strategy that may be used to provide a desired expression profile is the use of synthetic promoters containing cis-regulatory elements that drive the desired expression levels at the desired time and place in the plant. Cis-regulatory elements that can be used to alter gene expression in planta have been described in the scientific literature (Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

### 3. Transfer DNA

Nucleic acid molecules comprising transfer DNA (T-DNA) sequences can be used in the practice of the disclosure, e.g., to express editing reagents in plants, plant parts, or plant cells. For example, a construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a gRNA of the present disclosure into nuclear DNA genome of a host plant (e.g., a pea plant).

### 4. Regulatory Signal

Construct described herein may contain regulatory signals, including, but not limited to, transcriptional initiation sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

### 5. Reporter Genes / Selectable Marker Genes

Reporter genes or selectable marker genes may be included in the expression cassettes of the present invention. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. Pat. Application Serial Nos 10/004,357 and 10/427,692); phosphinothricin (DeBlock, et al., (1987) EMBO J. 6:2513-2518), herein incorporated by reference in their entirety.

Selectable marker genes include genes encoding antibiotic resistance, such as those encoding neomycin phosphotransferase II (NEO), spectinomycin/streptinomycin resistance (SpcR, AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

Herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Accl-S1, Accl-S2 and Accl-S3. Herbicides can also inhibit photosynthesis, including triazine (psbA and 1s+ genes) or benzonitrile (nitrilase gene). Further, such selectable markers can include positive selection markers such as phosphomannose isomerase (PMI) enzyme.

Selectable marker genes can further include, but are not limited to genes encoding: 2,4-D; SpcR; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). Selectable marker genes can further include genes encoding resistance to: chloramphenicol; methotrexate; hygromycin; spectinomycin; bromoxynil; glyphosate; and phosphinothricin.

Other selectable marker genes that could be employed on the expression constructs disclosed herein include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414), red fluorescent protein (DsRFP, RFP, etc), beta-galactosidase, and the maize genes encoding for saponin production (Ludwig, et al., (1990) Science 247:449), and the like (See Sambrook, et al., Molecular Cloning: A Laboratory Manual, Third Edition, Cold Spring Harbor Press, N.Y., 2001), herein incorporated by reference in their entirety. The above list of selectable marker genes is not meant to be limiting. Any reporter or selectable marker gene are encompassed by the present disclosure.

### 6. Terminator

A transcription terminator may also be included in the expression cassettes of the present invention. Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

### 7. Vector

Disclosed herein are vectors containing constructs (e.g., recombinant DNA constructs encoding editing reagents) of the present disclosure. As used herein, “vector” refers to a nucleotide molecule (e.g., a plasmid, cosmid), bacterial phage, or virus for introducing a nucleotide construct, for example, a recombinant DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance. In some embodiments, provided herein are expression cassettes located on a vector comprising gRNA sequence specific for at least one BAS gene or a regulatory region of the BAS gene.

In some embodiments, a vector is a plasmid containing a recombinant DNA construct of the present disclosure. For example, the present disclosure may provide a plasmid containing a recombinant DNA construct that comprises a gRNA to drive mutations at the locus of at least one BAS gene or the regulatory region of the BAS gene.

In some embodiments, a vector is a recombinant virus containing a recombinant DNA construct of the present disclosure. For example, the present disclosure may provide a recombinant virus containing a recombinant DNA construct that comprises a gRNA, wherein the gRNA can drive mutations at the locus of at least one BAS gene or the regulatory region of the BAS gene. A recombinant virus described herein can be a recombinant lentivirus, a recombinant retrovirus, a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

### 8. Cells

Also provided herein are cells comprising the reagent (e.g., editing reagent, e.g., nuclease, gRNA), the system (e.g., gene editing system), the construct (e.g., expression cassette), and/or the vector of the present disclosure for introducing mutations into at least one BAS gene and/or a regulatory region of the BAS gene. The cell can be a plant cell, a bacterial cell, and a fungal cell. The cell can be a bacterium, e.g., an Agrobacterium tumefaciens, containing the gRNA targeting at least one BAS gene and/or a regulatory region of the BAS gene and driving mutations at the target site of interest. The cells of the present disclosure may be grown, or have been grown, in a cell culture.

### C. Decreasing Saponin Content and/or Improving Flavor Characteristics in Plants

The methods of the present disclosure, by introducing a mutation that decreases BAS activity, e.g., comprising one or more insertions, substitutions, or deletions in at least one native BAS gene or homolog or in a regulatory region of such BAS gene or homolog in plants, plant parts, or plant cells and/or regenerating plants from transformed cells, can decrease saponin levels in the plants, plant parts (e.g., seeds, fruit), population of plants or plant parts, or plant products (e.g., plant protein composition) as compared to a control plant, plant part, population, or plant product, e.g., without such mutation.

A control plant or plant part can be a plant or plant part to which a mutation provided herein has not been introduced, e.g., by methods of the present disclosure. Thus, a control plant, plant part, population, or plant product may express a native (e.g., wild-type) BAS gene endogenously or transgenically, and/or may have a wild-type BAS activity. A control plant, plant part, or population of plants or plant parts of the present disclosure may be grown under the same environmental conditions (e.g., same or similar temperature, humidity, air quality, soil quality, water quality, and/or pH conditions) as a plant, plant part, or population of plants or plant parts produced according to the methods of the present disclosure. The methods provided herein can decrease saponin in a plant, plant part, population of plants or plant parts, or plant product as compared to a control plant, plant part, or plant product, when the plant, plant part, or plant population of the present disclosure is grown under the same environmental conditions as the control plant, plant part, or population.

In some embodiments, the methods provided herein can decrease saponin content in the plant, plant part, population of plants or plant parts, and/or plant product by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100% as compared to a control plant, plant part, population, or plant product. In specific embodiments, the methods provided herein decrease saponin content in the plant, plant part, population of plants or plant parts, and/or plant product by about 75-100%, at least about 75%, or at least about 97% as compared to a control plant, plant part, population, or plant product. In specific embodiments, the seeds of the plant, plant part, population of plants or plant parts, or the population of seeds produced by the methods provided herein contain from about 0 mg/g to about 0.8 mg/g of total saponins, and/or from about 0 mg/g to about 0.6 mg/g of DDMP saponins. Saponin levels in a plant, plant part, population of plants or plant parts, or plant product can be measured by any standard methods of measuring or estimating the amount of saponins in a sample. For example, saponin levels can be determined by using colorimetry, high performance liquid chromatography (HPLC), mass spectrometry, or liquid chromatography and tandem mass spectrometry (e.g., LC-MS/MS).

Further, the methods of the present disclosure can improve flavor characteristics of the plant, plant part (e.g., seeds, fruits), population of plants or plant parts, and plant product (e.g., plant protein compositions), which may result from reduced saponin content, as compared to a control plant, plant part, population, or plant product, e.g., without the mutation. Saponin content that contributes to flavor characteristics e.g., bitterness or off-flavor of a plant, plant part, population of plants or plant parts, or plant product can be quantified by using colorimetry, high performance liquid chromatography (HPLC), mass spectrometry, or liquid chromatography and tandem mass spectrometry (e.g., LC-MS/MS). To correlate these instrumental measurements to consumer perception, two major methods of sensory evaluation are used: consumer testing and descriptive analysis. Consumer testing includes subjective data about the preferences of a large group of untrained tasters (usually more than 100 panelists), while descriptive analysis includes questionnaires for a panel of 8-12 trained tasters who are able to rate specific attributes related to flavor or aroma. In certain instances, the methods provided herein can improve flavor characteristics of a plant, plant part, population of plants or plant parts, or plant product (e.g., plant protein composition) by a flavor panel experiment. Such flavor panel experiment may use instrumental measurements, sensory testing, or a combination thereof. Plant, plant part, or plant product (e.g., plant protein composition) that scores higher (as compared to a suitable control) in such flavor panel experiments can be considered to have improved flavor characteristics. For example, the methods provided herein can improve flavor panel experiment scores of the plant, plant part, population, or plant product of the present disclosure, e.g., comprising a mutation that decreases BAS activity, e.g., comprising one or more insertions, substitutions, or deletions in at least one native BAS gene or homolog or in a regulatory region of such BAS gene or homolog, compared to a control plant, plant part, population, or plant product (e.g., without the mutation), and thus can be considered to improve flavor characteristics of the plant, plant part, population, or plant product (e.g., plant protein composition) compared to the control plant, plant part, population, or plant product.

### D. Plants, Plant Parts, and Plant Products Produced by Present Methods

The present disclosure provides plants, plant parts, a population of plants and plant parts, and plant products produced according to the methods provided herein. Such plants, plant parts, and plant products can have reduced BAS activity compared to a control plant, plant part, or plant product. In the population of plants or plant parts provided herein, having altered BAS level or activity relative to a control population, not all individual plants or plant parts need to have altered (e.g., decreased) BAS level or activity, genetic mutation that cause altered (e.g., decreased) BAS level or activity, or phenotypes caused by the altered (e.g., decreased) BAS activity (e.g., decreased saponin content, improved flavor characteristics). In specific embodiments at least 80%, 81%, 82%, 83%, 84%, 85%, 86%, 87%, 88%, 89%, 90%, 91%, 92%, 93%, 94%, 95%, 96%, 97%, 98%, 99%, or more plants within a given plant population have a mutation that alters the BAS level or activity.

A “plant part” produced according to the methods described herein can include any part of a plant, including seeds (e.g., a representative sample of seeds), plant cells, embryos, pollen, ovules, leaves, flowers, branches, fruit, kernels, ears, cobs, husks, stalks, roots, root tips, anthers, plant protoplasts, plant cell tissue cultures from which plants can be regenerated, plant calli, plant clumps, juice, pulp, nectar, stems, branches, and bark. A “plant product” produced according to the methods described herein can include any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein.

A “protein product” or “protein composition” obtained from the plants or plant parts produced according to the methods provided herein can include any protein composition or product isolated, extracted, and/or produced from plants or plant parts (e.g., seed) and includes isolates, concentrates, and flours, e.g., soy protein composition, soy protein concentrate (SPC), soy protein isolate (SPI), soy flour, flake, white flake, texturized vegetable protein (TVP), or textured soy protein (TSP)). Plant protein compositions obtained according to the methods provided herein can be a concentrated protein solution (e.g., soybean protein concentrate solution) in which the protein is in a higher concentration than the protein in the plant from which the protein composition is derived. The protein composition can comprise multiple proteins as a result of the extraction or isolation process. The plant protein composition can further comprise stabilizers, excipients, drying agents, desiccating agents, anti-caking agents, or any other ingredient to make the protein fit for the intended purpose. The protein composition can be a solid, liquid, gel, or aerosol and can be formulated as a powder. The protein composition can be extracted in a powder form from a plant and can be processed and produced in different ways, such as: (i) as an isolate - through the process of wet fractionation, which has the highest protein concentration; (ii) as a concentrate - through the process of dry fractionation, which are lower in protein concentration; and/or (iii) in textured form -when it is used in food products as a substitute for other products, such as meat substitution (e.g. a “meat” patty). In specific embodiments, the plant protein compositions provided herein are obtained from a soybean (Glycine max) plant or plant part produced according to the methods of the present disclosure, e.g., a soybean plant or plant part to which a mutation that decreases BAS activity, e.g., one or more insertions, substitutions, or deletions is introduced into at least one native BAS gene or homolog or into a regulatory region of such BAS gene or homolog.

Food and/or beverage products of the present disclosure can contain plant compositions, e.g., plant protein compositions of the present disclosure. Food and/or beverage products of the present disclosure can include shakes (e.g., protein shakes), health drinks, alternative meat products (e.g., meatless burger patties, meatless sausages), alternative egg products (e.g., eggless mayo), non-dairy products (e.g., non-dairy whipped toppings, non-dairy milk, non-dairy creamer, non-dairy milk shakes, non-diary ice cream), energy bars (e.g., protein energy bars), infant formula, baby foods, cereals, baked goods, edamame, tofu, tempeh, and condiments.

Plant parts (e.g., seeds) and plant products (e.g., plant biomass, seed compositions, protein compositions, food and/or beverage products) produced by the methods provided herein can be meant for consumption by agricultural animals or for use as feed in an agriculture or aquaculture system. In specific embodiments, plant parts and plant products produced according to the methods provided herein include animal feed (e.g., roughages - forage, hay, silage; concentrates - cereal grains, soybean cake) intended for consumption by bovine, porcine, poultry, lambs, goats, or any other agricultural animal. In some embodiments, plant parts and plant products produced according to the methods include aquaculture feed for any type of fish or aquatic animal in a farmed or wild environment including, without limitation, trout, carp, catfish, salmon, tilapia, crab, lobster, shrimp, oysters, clams, mussels, and scallops.

The plants, plant parts, population of plants or plant parts, and plant products, including plant protein compositions and plant-based food/beverage products produced according to the methods of the present disclosure can contain a mutation that decreases BAS activity, e.g., one or more insertions, substitutions, or deletions in at least one native BAS gene or homolog or in a regulatory region of such BAS gene or homolog. The plants, plant parts, population of plants or plant parts, and plant products produced according to the methods of the present disclosure can have reduced BAS activity, reduced expression level of the BAS gene or homolog, reduced expression level of the BAS protein (e.g., the full-length BAS protein) encoded by the BAS gene, loss of function or reduced function or activity of the BAS protein encoded by the BAS gene, reduced saponin levels, and/or improved flavor characteristics compared to a control plant, plant part, population, or plant product, e.g., without the mutation, comprising a native (e.g., wild-type) BAS gene or BAS protein, or comprising wild-type BAS activity.

### E. Transformation of Plants

Provided herein are methods for transforming plants or plant parts by introducing into the plants or plant parts one or more mutations (e.g., insertions, substitutions, and/or deletions) to at least one BAS gene and/or a regulatory region of the BAS gene. The methods can comprise introducing a system (e.g., a gene editing system), reagents (e.g., editing reagents), or a construct for introducing mutations at the target site of interest.

The term “transform” or “transformation” as used herein refers to any method used to introduce genetic mutations (e.g., insertions substitutions, or deletions in the genome), polypeptides, or polynucleotides into plant cells. For purpose of the present disclosure, the transformation can be “stable transformation”, wherein the one or more mutations (e.g., in at least one BAS gene and/or a regulatory region of the BAS gene) or the transformation constructs (e.g., a construct comprising a nucleic acid molecule encoding a gRNA and/or a nuclease for use in the methods of the present invention) are introduced into a host (e.g., a host plant, plant part, plant cell, etc.), integrate into the genome of the host, and are capable of being inherited by the progeny thereof; or “transient transformation”, wherein the one or more mutations (e.g., in at least one BAS gene and/or a regulatory region of the BAS gene) or the transformation constructs (e.g., a construct comprising a gRNA and/or a gene encoding a nuclease for use in the methods of the present invention) are introduced into a host (e.g., a host plant, plant part, plant cell, etc.) and expressed temporarily. The methods disclosed herein can also be used for insertion of heterologous genes and/or modification of native plant gene expression to achieve desirable plant traits, e.g., decreased saponin content.

Any mutation or any polynucleotide of interest (e.g., editing reagents, e.g., a nuclease and a guide RNA) can be introduced into a plant, plant part, plant cell or organelle, or plant embryo by a variety of means of transformation, including mutagenesis by contacting the plant, plant part, plant cell, organelle, or plant embryo with a mutagen (e.g., EMS, ENU), microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration [see, for example, U.S. Pat. Nos. 4,945,050; U.S. Patent No. 5,879,918; U.S. Pat. No. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D′Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens)]; all of which are herein incorporated by reference.

Agrobacterium-and biolistic-mediated transformation remain the two predominantly employed approaches for transforming a plant or plant part. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, viral infection, Agrobacterium and viral mediated (Caulimoriviruses, Geminiviruses, RNA plant viruses), liposome mediated and the like. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

More than one polynucleotides of interest can be introduced into the plant, plant cell, plant organelle, or plant embryo simultaneously or sequentially. For example, different editing reagents, e.g., nuclease polypeptides (or encoding nucleic acid), guide RNAs (or DNA molecules encoding the guide RNAs), donor polynucleotide(s), and/or repair templates can be introduced into the plant cell, organelle, or plant embryo simultaneously or sequentially. The amount or ratio of more than one polynucleotides of interest, or molecules encoded therein, can be adjusted by adjusting the amount or concentration of the polynucleotides and/or timing and dosage of introducing the polynucleotides into the plant or plant part. For example, the ratio of the nuclease (or encoding nucleic acid) to the guide RNA(s) (or encoding DNA) to be introduced into plants or plant parts generally will be about stoichiometric such that the two components can form an RNA-protein complex with the target DNA. In one embodiment, DNA encoding a nuclease and DNA encoding a guide RNA are delivered together within a plasmid vector.

Alteration of the BAS level or activity in plants, plant parts, or plant cells may also be achieved through the use of transposable element technologies to alter gene expression. It is well understood that transposable elements can alter the expression of nearby DNA (McGinnis et al. (1983) Cell 34:75-84). Alteration of the BAS level or activity may be achieved by inserting a transposable element into at least one BAS gene and/or a regulatory region of the BAS gene.

The cells that have been transformed may be grown into plants (i.e., cultured) in accordance with conventional ways. See, for example, McCormick et al. (1986) Plant Cell Reports 5:81-84. In this manner, the present invention provides transformed plants or plant parts, transformed seed (also referred to as “transgenic seed”) or transformed plant progenies having a nucleic acid modification stably incorporated into their genome.

The present invention may be used for transformation of any plant species, e.g., both monocots and dicots (including legumes). Plants or plant parts to be transformed according to the methods disclosed herein can be a legume, i.e., a plant belonging to the family Fabaceae (or Leguminosae), or a part (e.g., fruit or seed) of such a plant. When used as a dry grain, the seed of a legume is also called a pulse. Examples of legume include, without limitation, soybean (Glycine max), beans (Phaseolus spp.), common bean (Phaseolus vulgaris), fava bean (Vicia faba), mung bean (Vigna radiata), pea (Pisum sativum), chickpea (Cicer arietinum), peanut (Arachis hypogaea), lentils (Lens culinaris, Lens esculenta), lupins (Lupinus spp.), white lupin (Lupinus albus), mesquite (Prosopis spp.), carob (Ceratonia siliqua.), tamarind (Tamarindus indica), alfalfa (Medicago sativa), barrel medic (Medicago truncatula), birdsfood trefoil (Lotus japonicus), licorice (Glycyrrhiza glabra), and clover (Trifolium spp.). In specific embodiments, a plant or plant part to be transformed according to the methods of the present disclosure is Glycine max or a part of Glycine max. Additionally, a plant or plant part to be transformed according to the methods present disclosure can be a crop plant or part of a crop plant, including legumes. Examples of crop plants include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana spp., e.g., Nicotiana tabacum, Nicotiana sylvestris), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), grapes (Vitis vinifera, Vitis riparia), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, a plant or plant part of the present disclosure can be an oilseed plant (e.g., canola (Brassica napus), cotton (Gossypium sp.), camelina (Camelina sativa) and sunflower (Helianthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), or hemp (Cannabis sativa).

The embodiments disclosed herein are not limited to certain methods of introducing nucleic acids into a plant and are not limited to certain forms or structures that the introduced nucleic acids take. Any method of transforming a cell of a plant described herein with mutations, polynucleotides, or polypeptides are also incorporated into the teachings of this innovation. For example, one of ordinary skill in the art will realize that the use of particle bombardment (e.g. using a gene-gun), Agrobacterium infection and/or infection by other bacterial species capable of transferring DNA into plants (e.g., Ochrobactrum sp., Ensifer sp., Rhizobium sp.), viral infection, and other techniques can be used to deliver mutations, polynucleotides, or polypeptides into a plant, plant part, or plant cell described herein.

The present disclosure provides plants and plant parts transformed according to the methods of the present disclosure. Transformed plant parts of the invention include plant cells, plant protoplasts, plant cell tissue cultures from which plants can be regenerated, plant calli, plant clumps, and plant cells that are intact in plants or parts of plants such as embryos, pollen, ovules, seeds, grains, leaves, flowers, branches, fruit, kernels, ears, cobs, husks, stalks, roots, root tips, anthers, and the like. Progeny, variants, and mutants of the regenerated plants are also included within the scope of the disclosure, provided that these parts comprise the introduced mutations, polynucleotides, or polypeptides.

### F. Breeding of Plants

Also disclosed herein are methods for breeding a plant, such as a plant which contains (i) a mutation that decreases the BAS activity, e.g., one or more insertions, substitutions, or deletions in at least one native BAS gene or homolog or in a regulatory region of such BAS gene or homolog, (ii) editing reagents, e.g., a polynucleotide encoding a guide RNA specific to at least one BAS gene or homolog or in a regulatory region of such BAS gene or homolog, and/or (iii) a polynucleotide comprising a mutated BAS gene or a BAS gene with a mutated regulatory region of a BAS gene. A plant containing the one or more mutations or the polynucleotide of the present disclosure may be regenerated from a plant cell or plant part, wherein the genome of the plant cell or plant part is genetically-modified to contain the one or more mutations or the polynucleotide of the present disclosure. Using conventional breeding techniques or self-pollination, one or more seeds may be produced from the plant that contains the one or more mutations or the polynucleotide of the present disclosure. Such a seed, and the resulting progeny plant grown from such a seed, may contain the one or more mutations or the polynucleotide of the present disclosure, and therefore may be transgenic. Progeny plants are plants having a genetic modification to contain the one or more mutations or the polynucleotide of the present disclosure, which descended from the original plant having modification to contain the one or more mutations or the polynucleotide of the present disclosure. Seeds produced using such a plant of the invention can be harvested and used to grow generations of plants having genetic modification to contain the one or more mutations or the polynucleotide of the present disclosure, e.g., progeny plants, of the invention, comprising the polynucleotide and optionally expressing a gene of agronomic interest (e.g., herbicide resistance gene).

Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

Methods disclosed herein include conferring desired traits (e.g., increased sucrose content) to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain nonlimiting embodiments, the trait is conferred to the plant by introducing a nucleic acid sequence (e.g. using plant transformation methods) that encodes production of a certain protein by the plant. In certain nonlimiting embodiments, the desired trait is conferred to a plant by causing a null mutation in the plant’s genome (e.g. when the desired trait is reduced expression or no expression of a certain trait). In certain nonlimiting embodiments, the desired trait is conferred to a plant by crossing two plants to create offspring that express the desired trait. It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

In certain embodiments, a user can combine the teachings herein with high-density molecular marker profiles spanning substantially the entire genome of a plant to estimate the value of selecting certain candidates in a breeding program in a process commonly known as genome selection. Breeding of soybean plants having low saponin trait is further described below.

### V. Nucleic Acid Molecules, Constructs, and Cells Comprising Mutated BAS Gene or Mutated Regulatory Region of BAS Gene

### A. Nucleic Acid Molecules

Nucleic acid molecules are provided herein comprising a mutated genomic sequence that decreases BAS activity in a plant or plant part. The nucleic acid molecule can comprise any nucleic acid sequence that decreases BAS activity in a plant or plant part including those described herein, e.g., an altered (e.g., mutated, alternatively spliced) nucleic acid sequence of a BAS gene, a regulatory region of the BAS gene, or a BAS gene transcript, encoding an altered (e.g., mutated, alternatively spliced, truncated) BAS protein relative to a corresponding native BAS gene or BAS protein. Such nucleic acid molecules may be present in, or obtained from, a plant cell, plant part, or plant of the present disclosure, or may be obtained by the methods described herein, e.g., by introducing one or more mutations into at least one BAS gene or a regulatory region of the BAS gene and/or by introducing editing reagents targeting a site of interest in at least one BAS gene or a regulatory region of the BAS gene in a plant or plant part. The nucleic acid molecule described herein can encode an altered (e.g., mutated, truncated, alternatively spliced) BAS protein that can comprise a different amino acid sequence from a native BAS protein (e.g., without mutations). The nucleic acid molecule described herein can encode a BAS protein with reduced function or loss-of-function of beta-amyrin synthsase, e.g., the ability to convert 2, 3-oxidosqualene to beta-amyrin, as compared to a native BAS protein (e.g., without mutations). The mutated sequence, e.g., altered nucleic acid sequence of the BAS gene and/or the regulatory region of the BAS gene can result in reduced expression levels of the BAS gene or BAS protein (e.g., full-length BAS protein, functional BAS protein), as compared to a native BAS gene and/or a regulatory region of a native BAS gene e.g., without mutations.

The nucleic acid molecule provided herein can encode a BAS protein and comprise one or more (e.g., about 1, 2, 3, 4, 5, 6, 7, 8, 9, 10, 11, 12, 13, 14, 15, 16, 17, 18, 19, 20, or more) insertions, substitutions, and/or deletions in a BAS gene or homolog and/or a regulatory region of the BAS gene or homolog compared to a corresponding native a BAS gene or homolog and/or a regulatory region of the native BAS gene or homolog. The nucleic acid molecule may comprise an in-frame mutation (e.g., missense mutation) or a frameshift (out-of-frame) mutation of the BAS gene or homolog.

The mutation in the nucleic acid molecule provided herein can be located in Glycine max BAS genes, such as a Glycine max BAS1 gene, a Glycine max BAS2 gene, a Glycine max BAS3 gene, a Glycine max BAS4 gene, a Glycine max BAS5 gene and/or a regulatory region of such one or more Glycine max BAS genes and decrease BAS activity of an encoded protein. In some embodiments, the mutation is located in a Glycine max BAS1 gene and/or a regulatory region of the Glycine max BAS1 gene. In some embodiments, the mutation is located in a BAS gene or homolog thereof comprising a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of any one of SEQ ID NOs: 1-5 and 38 and encoding a polypeptide that retains BAS activity, for example the nucleic acid sequence of any one of SEQ ID NOs: 1-5 and 38; and/or a regulatory region of the BAS gene or homolog thereof comprising such nucleic acid sequence. Additionally, the mutation can be located in a BAS gene or homolog thereof encoding a polypeptide comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of any one of SEQ ID NOs: 6-10 and retaining BAS activity, for example a polypeptide comprising the amino acid sequence of any one of SEQ ID NOs: 6-10; and/or a regulatory region of the BAS gene or homolog thereof encoding such polypeptide. In specific embodiments, the mutation that decreases the BAS activity is located in a BAS1 gene or homolog thereof comprising a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a nucleic acid sequence of SEQ ID NO: 1 or 38 and encoding a polypeptide that retains BAS activity, for example the nucleic acid sequence of SEQ ID NO: 1 or 38, and/or a regulatory region of the BAS1 gene or homolog thereof comprising such nucleic acid sequence. Additionally, the mutation can be located in a BAS1 gene or homolog thereof encoding a polypeptide comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to an amino acid sequence of SEQ ID NO: 6 and retaining BAS activity, for example a polypeptide comprising an amino acid sequence of SEQ ID NO: 6; and/or a regulatory region of the BAS1 gene or homolog thereof encoding such polypeptide.

The mutation in the nucleic acid molecule provided herein can be at least one (e.g., about 1, 2, 3, 4, 5, 6, 7, 8, 9, 10, 11, 12, 13, 14, 15, 16, 17, 18, 19, 20, or more) insertion, substitution, or deletion located in a nucleic acid region of exon 2, 4, and/or 7 of the Glycine max BAS1 gene. The mutation in the nucleic acid molecule provided herein can comprise a deletion of about 4-78 nucleotides at least partially in the nucleic acid region of exon 7 of the Glycine max BAS1 gene. The nucleic acid molecule of the present disclosure can have at least 80% identity to a nucleic acid sequence of (i) SEQ ID NO: 1 consisting of a deletion of nucleotides 4191 through 4195 thereof (at chr07:137242 to 137246 in the Glycine max BAS1 gene), (ii) SEQ ID NO: 1 consisting of a deletion of nucleotides 4190 through 4199 thereof, (iii) SEQ ID NO: 1 consisting of a deletion of nucleotides 4171 through 4198 thereof, (iv) SEQ ID NO: 1 consisting of a deletion of nucleotides 4187 through 4190 thereof, (v) SEQ ID NO: 1 consisting of a deletion of nucleotides 4189 through 4198 thereof, (vi) SEQ ID NO: 1 consisting of a deletion of nucleotides 4120 through 4197 thereof, (vii) SEQ ID NO: 1 consisting of a deletion of nucleotides 4187 through 4191 thereof, (viii) SEQ ID NO: 1 consisting of a deletion of nucleotides 4188 through 4195 thereof, and/or (ix) SEQ ID NO: 1 consisting of a deletion of nucleotides 4187 through 4194 thereof), and encode a BAS protein having decreased level or activity compared to a BAS protein encoded by the native BAS gene. For example, the nucleic acid molecule of the present disclosure can comprise a nucleic acid sequence of: (i) SEQ ID NO: 1 with a deletion of nucleotides 4191 through 4195 of SEQ ID NO: 1, (ii) SEQ ID NO: 1 with a deletion of nucleotides 4190 through 4199, (iii) SEQ ID NO: 1 with a deletion of nucleotides 4171 through 4198, (iv) SEQ ID NO: 1 with a deletion of nucleotides 4187 through 4190, (v) SEQ ID NO: 1 with a deletion of nucleotides 4189 through 4198, (vi) SEQ ID NO: 1 with a deletion of nucleotides 4120 through 4197, (vii) SEQ ID NO: 1 with a deletion of nucleotides 4187 through 4191, (viii) SEQ ID NO: 1 with a deletion of nucleotides 4188 through 4195, (ix) SEQ ID NO: 1 with a deletion of nucleotides 4187 through 4194.

The mutation in the nucleic acid molecule provided herein can comprise a substitution or deletion in the nucleic acid region of exon 2 and/or 4 of the Glycine max BAS1 gene. For example, the nucleic acid molecule of the present disclosure can have at least 80% identity to a nucleic acid sequence of (i) SEQ ID NO: 1 consisting of a G to A substitution of nucleotide 3564 thereof or SEQ ID NO: 38 consisting of a G to A substitution of nucleotide 3750 thereof (chr07: 136615) or (ii) SEQ ID NO: 1 consisting of an A to T substitution of nucleotide 374 thereof or SEQ ID NO: 38 consisting of an A to T substitution of nucleotide 560 thereof (chr07:133425), and encode a BAS protein having decreased level or activity compared to a BAS protein encoded by the native BAS gene. For example, the nucleic acid molecule of the present disclosure can comprise a nucleic acid sequence of: (i) SEQ ID NO: 1 consisting of a G to A substitution of nucleotide 3564 thereof (chr07:136615) or (ii) SEQ ID NO: 1 consisting of an A to T substitution of nucleotide 374 thereof (chr07:133425). Further, the nucleic acid molecule of the present disclosure can comprise a nucleic acid sequence that encodes a polynucleotide comprising (i) an amino acid sequence having at least 80% sequence identity to an amino acid sequence of SEQ ID NO: 6 with a G to E substitution of amino acid 220 or an R to W substitution of amino acid 100; or (ii) an amino acid sequence of SEQ ID NO: 6 with a G to E substitution of amino acid 220 or an R to W substitution of amino acid 100, and having decreased level or activity compared to a BAS protein without mutation.

In some embodiments, the nucleic acid molecules described herein do not comprise a regulatory region (e.g., a promoter region) of a BAS gene or homolog. Alternatively, the nucleic acid molecules can comprise the regulatory region (e.g., promoter region) of the BAS gene or homolog. The regulatory region (e.g., promoter regions) in the nucleic acid molecule can comprise one or more (e.g., about 1, 2, 3, 4, 5, 6, 7, 8, 9, 10, 11, 12, 13, 14, 15, 16, 17, 18, 19, 20, or more) insertions, substitutions, and/or deletions. The one or more insertions, substitutions, and/or deletions in the regulatory region of the BAS gene or homolog can alter expression level or manner of the BAS gene or homolog. For example, the one or more insertions, substitutions, and/or deletions in the promoter region of the BAS gene or homolog can alter the transcription initiation activity of the promoter. The modified promoter can alter (e.g., reduce) transcription of the operably linked nucleic acid molecule, initiate transcription in a developmentally-regulated manner, initiate transcription in a cell-specific, cell-preferred, tissue-specific, or tissue-preferred manner, or initiate transcription in an inducible manner. The modified promoter can comprise a deletion, a substitution, or an insertion, e.g., introduction of a heterologous promoter sequence, a cis-acting factor, a motif or a partial sequence from any promoter, including those described elsewhere in the present disclosure, to confer an altered (e.g., reduced) transcription initiation function according to the present disclosure.

The nucleic acid molecule described herein can comprise one or more insertions, substitutions, and/or deletions in the regulatory region (e.g., promoter region) of the BAS gene as well as in the exon/intron region of the BAS gene.

### B. DNA Constructs, Vectors, and Cells

The nucleic acid molecules encoding molecules of interest of the present invention can be assembled within a DNA construct with an operably-linked promoter. When transiently or stably transformed with such DNA construct, a plant, plant part, or plant cell can express or accumulate polynucleotides comprising an altered (e.g., mutated, alternatively spliced) sequence of a BAS gene or a BAS gene transcript, or a BAS protein encoded by the polynucleotides. For example, the nucleic acid molecules described herein can be provided in expression cassettes or expression constructs along with a promoter sequence of interest, typically a heterologous promoter sequence, for expression in the plant of interest. By “heterologous promoter sequence” is intended a sequence that is not naturally operably linked with the nucleic acid molecule of interest. For instance, a 2×35s promoter, a native promoter, or a promoter (native or heterologous) comprising an exogenous or synthetic motif sequence may be operably linked to the nucleic acid sequences comprising an altered (e.g., mutated, alternatively spliced) sequence of a BAS gene or a BAS gene transcript. The BAS-encoding nucleic acid sequences or the promoter sequence may each be homologous, native, heterologous, or foreign to the plant host. It is recognized that the heterologous promoter may also drive expression of its homologous or native nucleic acid sequence. In this case, the transformed plant will have a change in phenotype.

Accordingly, the present disclosure provides DNA constructs comprising, in operable linkage, a promoter that is functional in a plant cell, and a nucleic acid molecule of the present disclosure, e.g., comprising an altered nucleic acid sequence of a BAS gene or a BAS gene transcript relative to a corresponding native nucleic acid sequence. When the DNA construct or nucleic acid molecule provided herein is introduced in a plant, plant part, or plant cell, BAS activity can be reduced, expression levels of the BAS gene or homolog can be decreased, BAS protein level or activity can be decreased, beta-amyrin levels can be decreased, saponin content can be decreased, and/or flavor characteristics can be improved in the plant, plant part, or plant cell as compared to a control plant, plant part, or plant cell, e.g., a plant, plant part, or plant cell to which the construct or the nucleic acid molecule of the present disclosure are not introduced. The DNA construct can further comprise, in operable linkage, a reporter construct (e.g., GFP, a HA tag).

Provided herein are vectors comprising the nucleic acid molecule and/or the DNA construct of the present disclosure comprising an altered nucleic acid sequence of the BAS gene, the regulatory region of the BAS gene, and/or the BAS gene transcript. Any vectors can be used, including the vectors described elsewhere in the present disclosure.

Also provided herein are cells comprising the nucleic acid molecule, the DNA construct, and/or the vector of the present disclosure comprising an altered nucleic acid sequence of the BAS gene, the regulatory region of the BAS gene, and/or the BAS gene transcript. The cell can be a plant cell, a bacterial cell, and a fungal cell. The cell can be a bacterium, e.g., an Agrobacterium tumefaciens, containing the nucleic acid molecule, the DNA construct, or the vector of the present disclosure. The cells of the present disclosure may be grown, or have been grown, in a cell culture.

Also provided herein are methods for generating a plant, plant part, plant cell, or a population of plants or plant parts (e.g., seeds) comprising decreased BAS activity, or a decreased saponin content, by introducing into the plant, plant part, or plant cell the nucleic acid molecule, the DNA construct, or the vector of the present disclosure. In some embodiments, the DNA construct is introduced into the plant by stable transformation. In other embodiments, the DNA construct is introduced into the plant by transient transformation. The present disclosure further provides plants, plant parts (juice, pulp, seed, fruit, flowers, nectar, embryos, pollen, ovules, leaves, stems, branches, bark, kernels, ears, cobs, husks, stalks, roots, root tips, anthers, etc.), population of plants or plant parts, or plant products (e.g., plant extract, plant concentrate, plant powder, plant protein, plant biomass, and food and beverage products) generated by the methods described herein.

### VI. Methods of Producing Low-Saponin Soybean Plants or Seeds

Provided herein are compositions and methods for generating low-saponin soybean plants or seeds. “Low-saponin” soybean plants or seeds, as used herein, refer to soybean plants or seeds having lower saponin content relative to control soybean plants or seeds. Control plants or seeds can be of a reference variety or a commonly available variety of plants or plant seeds. One skilled in the art can select an appropriate control. In certain cases, seeds of a reference variety of soybean cultivar may contain about 2.7-7.0 mg/g of total saponins, of which about 60-80% can be DDMP saponins (which is particularly astringent species of saponins). Low-saponin soybean plants or seeds provided herein can contain saponin at any amount that is lower than a reference variety of soybean cultivar. In specific embodiments, low-saponin soybean plants contain from about 0 mg/g to about 0.8 mg/g of total saponins, and/or from about 0 mg/g to about 0.6 mg/g of DDMP saponins.

### A. Methods of Producing a Population of Low-Saponin Plants or Seeds

The present disclosure provides a method of creating a population of low-saponin soybean plants or seeds. In certain aspects, the method provided herein uses a low-saponin marker, and comprises the steps of (a) genotyping a first population of soybean plants or seeds for the presence of at least one low-saponin marker that is within 20 centimorgans of at least one low-saponin quantitative trait locus (QTL) located within a genomic region 132866-141435 of chromosome 7 of a soybean genome, (b) selecting from the first population one or more soybean plants or seeds comprising one or more low-saponin alleles having the one or more low-saponin molecular markers, and (c) producing a second population of progeny soybean plants or seeds from the selected one or more soybean plants or plants grown from the selected seeds, such that the second population of progeny soybean plants or seeds comprises the one or more low-saponin alleles having the one or more low-saponin molecular markers, and the second population of progeny soybean plants or seeds comprises low-saponin content relative to a control population.

A “low-saponin” marker or a “low-saponin” QTL as used herein refers to a marker or a QTL associated with lower saponin in a plant, plant seed, or plant composition relative to a control plant, plant seed, or plant composition.

In some embodiments, the low-saponin QTL or marker is located in Glyma. 07g00I300, Glyma.08g225800, Glyma.03g121300, Glyma.03g121500, or Glyma.15g101800 of the soybean plants or seeds. In some embodiments, the low-saponin QTL is Gm07_137242, Gm07_133425, or Gm07_136615, as set forth in Table 1.

TABLE 1

Low saponin QTLs in soybean

| | | | | |
|---|---|---|---|---|
| QTLs | Position | Desired | Control | Gene ID |
| Gm07_137242 | Chr07:137242..137246 | - | GCTAC | Glyma.07g001300 |
| Gm07_133425 | Chr07:133425 | T | A | Glyma.07g001300 |
| Gm07_136615 | Chr07:136615 | A | G | Glyma.07g001300 |

QTLs that exhibit significant co-segregation with low-saponin phenotype are provided herein. In specific embodiments, plants or seeds comprising the low-saponin QTLs further comprise one or more allele associated with a low saponin content. In some embodiments, the one or more allele associated with a low saponin content is within 20 centimograns or within 10 centimorgans from one or more low-saponin QTLs. Low-saponin QTLs can be tracked during plant breeding or introgressed into a desired genetic background in order to provide plants exhibiting a low saponin content and, in specific embodiments, one or more other beneficial traits. In an aspect, this disclosure identifies QTL intervals that are associated with low saponin content in different soybean varieties described herein.

Low saponin markers of the present disclosure include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers. In a diploid organism such as soybeans, a marker genotype typically comprises two marker alleles at each locus. The marker allelic composition of each locus can be either homozygous or heterozygous. Homozygosity is a condition where both alleles at a locus are characterized by the same nucleotide sequence. Heterozygosity refers to different conditions of the gene at a locus.

Low-saponin markers can be simple sequence repeat markers (SSR, also referred to as simple sequence length polymorphisms (SSLPs)), amplified fragment length polymorphism (AFLP) markers, restriction fragment length polymorphism (RFLP) markers, RAPD markers, phenotypic markers, single nucleotide polymorphisms (SNPs), isozyme markers, deletion markers, microarray transcription profiles that are genetically linked to or correlated with alleles of a QTL of the present invention (Walton, Seed World 22-29 (July, 1993), Burow et al., Molecular Dissection of Complex Traits, 13-29, ed. Paterson, CRC Press, New York (1988)). Methods to isolate and identify such markers are known in the art. For example, locus-specific SSR markers can be obtained by screening a genomic library for microsatellite repeats, sequencing of “positive” clones, designing primers which flank the repeats, and amplifying genomic DNA with these primers. The size of the resulting amplification products can vary by integral numbers of the basic repeat unit. Polymorphisms comprising as little as a single nucleotide change can be assayed in a number of ways. For example, detection can be made by electrophoretic techniques including a single strand conformational polymorphism (Orita et al., 1989), denaturing gradient gel electrophoresis (Myers et al., 1985), cleavage fragment length polymorphisms (Life Technologies, Inc., Gathersberg, Md. 20877), or direct sequencing of amplified products. Once the polymorphic sequence difference is known, rapid assays can be designed for progeny testing, typically involving some version of PCR amplification of specific alleles (PASA, Sommer, et al., 1992), or PCR amplification of multiple specific alleles (PAMSA, Dutton and Sommer, 1991). PCR products can be radiolabeled, separated on denaturing polyacrylamide gels, and detected by autoradiography. Fragments with size differences > 4 bp can also be resolved on agarose gels, thus avoiding radioactivity.

A single nucleotide polymorphisms (SNP) occurs at a single nucleotide. SNPs are more stable than other classes of polymorphisms. Their spontaneous mutation rate is approximately 10-9 (Kornberg, DNA Replication, W. H. Freeman & Co., San Francisco (1980)). As SNPs result from sequence variation, new polymorphisms can be identified by sequencing random genomic or cDNA molecules. SNPs can also result from deletions, point mutations and insertions. That said, SNPs are also advantageous as markers since they are often diagnostic of “identity by descent” because they rarely arise from independent origins. Any single base alteration, whatever the cause, can be a SNP. SNPs occur at a greater frequency than other classes of polymorphisms and can be more readily identified. In the present disclosure, a SNP can represent a single indel event, which may consist of one or more base pairs, or a single nucleotide polymorphism.

In some embodiments, the low-saponin QTL comprises at least one SNP, and the at least one low-saponin marker comprises an allele of the at least one SNP. In some embodiments, the SNP contained in the low saponin QTL is a T or an A at position 133425 and/or an A or a G at position 136615 of chromosome 7 of the soybean genome. The T at position 133425 or the A at position 136615 of chromosome 7 of the soybean genome is associated with low-saponin content.

In some embodiments, the low-saponin QTL comprises a deletion marker. As used herein, a “deletion marker” refers to a deletion of a nucleotide region in the genome of plants or plant parts associated with a low-saponin phenotype. Plants or plant parts having genomes having the deletion marker can exhibit a low-saponin content by weight as compared to the plants and plant parts lacking the deletion marker. The deleted nucleotide region of a deletion marker can be a deletion of any number of consecutive nucleotides that is associated with a low-saponin phenotype. For example, the deletion can be 2-500 bp, 5-250 bp, 10-200 bp, 20-180 bp, 40-160 bp, 50-140 bp, 60-120 bp, 70-100 bp, 80-100 bp, 85-95 bp, or about 2 bp, 5 bp, 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 100 bp, 105 bp, 110 bp, 120 bp, 130 bp, 140 bp, 150 bp, 160 bp, 170 bp, 180 bp, 200 bp, 225 bp, 250 bp, 275 bp, 300 bp, 350 bp, 400 bp, 450 bp, or about 500 bp. In specific embodiments, the deletion maker can be wholly or at least partially within a gene. The deletion marker can be wholly or at least partially within an exon or intron of the gene. That is, the deletion marker can be a deletion of a nucleotide sequence entirely within a gene or spanning the 5′ end of the gene or the 3′ of the gene. In some embodiments, the deletion marker eliminates the start codon of a gene. The deletion marker can also account for removal of a signal peptide of a gene. In some embodiments, the deletion marker eliminates both the start codon and the signal peptide of a gene. The gene can be any gene in the genome. For example, the deletion marker can be wholly or at least partially within a beta-amyrin synthase (BAS) gene or regulatory region thereof. The BAS gene can be Glyma.07g001300, Glyma.08g225800, Glyma.03g121300, Glyma.03g121500, or Glyma.15g101800. In specific embodiments, the deletion marker comprises a deletion of a portion of exon 7 of the BAS gene. In further specific embodiments, the deletion marker comprises a deletion of positions Gm07_137242-137246 of a soybean genome.

The low-saponin QTLs disclosed herein can be an expression QTL (eQTL). As used herein an eQTL refers to a QTL that is associated with differential expression of a gene. In specific embodiments, when a QTL is present in the genome, a gene associated with the eQTL is has reduced expression. For example, the presence of an eQTL can eliminate or substantially eliminate expression of a gene.

In some embodiments, selecting from the first population one or more soybean plants or seeds is based on detection of the presence of an SNP or a haplotype associated with an low saponin phenotype. A “haplotype” as used herein refers to a plurality of SNPs. An low saponin haplotype can comprise low saponin alleles of two or more polymorphic loci (e.g., low-saponin loci) described herein.

In some embodiments, the genotyping according to the methods provided herein comprises analyzing the at least one SNP, haplotype, or deletion using an oligonucleotide probe comprising at least 15 nucleotides, wherein the oligonucleotide probe has at least 90% sequence identity to a sequence of the same number of contiguous nucleotides of a sense or antisense DNA strand in a region comprising or adjacent to the SNP or deletion in the soybean genome. For example, the oligonucleotide probe can comprise a nucleic acid sequence having at least 90% identity to a nucleic acid sequence of SEQ ID NOs: 17 and 21 or a nucleic acid sequence of SEQ ID NOs: 17 and 21 for detection of a low-saponin SNP marker (or a desirable SNP), or a nucleic acid sequence having at least 90% identity to a nucleic acid sequence of any one of SEQ ID NOs: 18 and 22 or a nucleic acid sequence of any one of SEQ ID NOs: 18 and 22 for detection of absence of a low-saponin SNP marker (or a undesirable SNP).

Additionally or alternatively, genotyping can comprise analyzing the SNP, haplotype, or deletion using a first primer and a second primer each comprising at least 15 nucleotides, using PCR or quantitative PCR. The first primer can have at least 90% sequence identity to a sequence of the same number of contiguous nucleotides of a sense DNA strand of a region comprising or adjacent to the at least one SNP, and the second primer can have at least 90% sequence identity to a sequence of the same number of contiguous nucleotides of an antisense DNA strand of the region comprising or adjacent to the at least one SNP. For example, the first and second primers can comprise (i) nucleic acid sequences having at least 90% identity to nucleic acid sequences of SEQ ID NOs: 13 and 14, or nucleic acid sequences of SEQ ID NOs: 13 and 14; or (ii) nucleic acid sequences having at least 90% identity to nucleic acid sequences of SEQ ID NOs: 15 and 16, or nucleic acid sequences of SEQ ID NOs: 15 and 16; (iii) nucleic acid sequences having at least 90% identity to nucleic acid sequences of SEQ ID NOs: 19 and 20, or nucleic acid sequences of SEQ ID NOs: 19 and 20 for detection of a low-saponin SNP.

For assaying a deletion marker, any method known in the art can be used to identify a region of the genome that is missing a given position, including but not limited to PCR, RFLP, probe-based detection methods, and sequencing methods, among others. For example, first and second primers comprising nucleic acid sequences having at least 90% identity to nucleic acid sequences of SEQ ID NOs: 13 and 14, or nucleic acid sequences of SEQ ID NOs: 13 and 14, can be used for detection of a low-saponin deletion marker.

In specific embodiments, the presence of low-saponin molecular markers in a plant, plant part, plant seed, plant composition, or plant/plant part population is associated with lower saponin content than corresponding plants, plant parts, plant seeds, or plant composition without the low-saponin molecular markers. For example, total saponin content or DDMP saponin content in a plant, plant part, plant seed, plant composition, or plant/plant part population comprising the low-saponin markers can be lower by at least 5%, 10%, 15%, 20%, 25%, 30%, 35%, 40%, 45%, 50%, 55%, 60%, 65%, 70%, 75%, 80%, 85%, 90%, 95%, or 99%; or about at least 5%, 10%, 15%, 20%, 25%, 30%, 35%, 40%, 45%, 50%, 55%, 60%, 65%, 70%, 75%, 80%, 85%, 90%, 95%, 99%, or 100%; or about 5-100%, 10-100%, 20-100%, 30-100%, 40-100%, 50-100%, 60-100%, 70-100%, 75-100%, 75-80%, 80-100%, 90-100%, or 97-100% as compared to a control plant, plant part, plant seed, plant composition, or plant/plant part population not comprising the low-saponin marker. A control plant, plant part, plant seed, plant composition, or plant/plant part population not comprising the low-saponin marker may contain about 2.7-7.0 mg/g of total saponins, of which about 60-80% can be DDMP saponins. A plant, plant part, plant seed, plant composition, or plant/plant part population comprising the low-saponin marker provided herein can contain total saponin content of less than about 2.7-7.0 mg/g (e.g., 6.5 mg/g or less, 6.0 mg/g or less, 5.5 mg/g or less, 5.0 mg/g or less, 4.5 mg/g or less, 4.0 mg/g or less, 3.5 mg/g or less, 3.0 mg/g or less, 2.7 mg/g or less, 2.5 mg/g or less, 2.0 mg/g or less, 1.5 mg/g or less, 1.0 mg/g or less, 0.5 mg/g or less), and/or DDMP saponin content of less than about 2.0-5.5 mg/g (e.g., 5.5 mg/g or less, 5.0 mg/g or less, 4.5 mg/g or less, 4.0 mg/g or less, 3.5 mg/g or less, 3.0 mg/g or less, 2.5 mg/g or less, 2.0 mg/g or less, 1.5 mg/g or less, 1.0 mg/g or less, 0.5 mg/g or less). In specific embodiments, a plant, plant part, plant seed, plant composition, or plant/plant part population comprising the low-saponin marker contains from about 0 mg/g to about 0.8 mg/g of total saponins, and/or from about 0 mg/g to about 0.6 mg/g of DDMP saponins.

Saponin content in a plant, plant part, or plant product can be measured by any standard methods of measuring or estimating the amount of saponins in a sample. For example, saponin content can be determined by using colorimetry, high performance liquid chromatography (HPLC), mass spectrometry, or liquid chromatography and tandem mass spectrometry (e.g., LC-MS/MS).

In some embodiments, the methods provided herein can produce low saponin soybean plants or seeds without a corresponding reduction or penalty in crop yield. The plants described in embodiments herein may have, for example, a yield in excess of 35 bushels per acre.

As disclosed herein, a soybean plant or seed refers to a plant, plant part, or seed of Glycine max (L). In specific embodiments, all chromosomal positions listed herein are identified relative to the reference genome published as the Williams 82 reference genome assembly (Wm82.a2.v1) that can be accessed at the website located at phytozome-next.jgi.doe.gov/info/Gmax_Wm82_a2_v1. See, Schmutz, J., Cannon, S., Schlueter, J. et al. Genome sequence of the palaeopolyploid soybean. Nature 463, 178-183 (2010). The wild perennial soybeans belong to the subgenus Glycine and have a wide array of genetic diversity. The cultivated soybean (Glycine max (L.) Merr.) and its wild annual progenitor (Glycine soja (Sieb. and Zucc.)) belong to the subgenus Soja. The methods described herein can be used in any soybean plant or seed, including but not limited to members of the genus Glycine, for example, Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine sp., Glycine stenophita, Glycine tabacina and Glycine tomentella.

### B. Methods of Introgressing a Low-Saponin QTL

Provided herein are methods for selection and introgression of a low-saponin QTL. The methods can comprise the steps of (a) crossing a first soybean plant comprising a low-saponin QTL with a second soybean plant of a different genotype to produce one or more progeny plants or seeds and (b) selecting a progeny plant or seed comprising a low-saponin allele of a polymorphic locus linked to the low-saponin QTL. The polymorphic locus associated with the QTL can be a chromosomal segment comprising a low-saponin marker within the genomic region 132866-141435 of soybean chromosome 7. In some embodiments, the low-saponin QTL is Gm07_137242, Gm07_133425, or Gm07_136615.

In some embodiments, the polymorphic locus associated with the low-saponin QTL comprises at least one single nucleotide polymorphisms (SNP), and the low-saponin marker comprises said at least one SNP. Selecting the progeny plant or seed from the population can be based on the presence of a low-saponin haplotype. In particular embodiments, a low-saponin haplotype comprises alleles of two or more polymorphic loci described herein. In some embodiments of the method, the SNP is a T or an A at position 133425 and/or an A or a G at position 136615 of chromosome 7 of the soybean genome, wherein the T at position 133425 or the A at position 136615 of chromosome 7 of the soybean genome is associated with low-saponin content.

In some embodiments, the polymorphic locus associated with the low-saponin QTL comprises a deletion marker. The deletion maker can be wholly or at least partially within a gene (e.g., an exon or intron of the gene). In some embodiments, the deletion marker is wholly or at least partially within a beta-amyrin synthase (BAS) gene or regulatory region thereof. The BAS gene can be Glyma.07g001300, Glyma.08g225800, Glyma.03g121300, Glyma.03g121500, or Glyma.15g101800. In specific embodiments, the deletion marker comprises a deletion of a portion of exon 7 of the BAS gene. In further specific embodiments, the deletion marker comprises a deletion of positions Gm07_137242-137246 of a soybean genome.

The low-saponin QTLs disclosed herein can be an expression QTL (eQTL).

In some embodiments, provided herein are methods for concurrently introgressing at least one or more, two or more, three or more, five or more, six or more, seven or more, eight or more, nine or more, ten or more, eleven or more, or twelve low-saponin QTLs, including those identified herein, to generate a population of low-saponin soybean plants or seeds. In one embodiment, the present disclosure provides a method for introgressing an allele of a polymorphic locus conferring a low-saponin phenotype.

The methods described herein can be applied to any soybean plant or seed, including but not limited to members of the genus Glycine, for example, Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine sp., Glycine stenophita, Glycine tabacina and Glycine tomentella. In specific embodiments, the low-saponin QTL of the present invention may be introduced into an agronomically elite Glycine max variety. An “agronomically elite” plant, as used herein refers to a plant having a culmination of distinguishable traits such as emergence, vigor, vegetative vigor, disease resistance, seed set, standability, threshability, and yield that allows a producer to harvest a commercially advantageous product.

### C. Detection/Identification of Low-Saponin Markers and QTLs

Genotyping, e.g., detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

In certain embodiments of the method described herein, genotyping comprises assaying a single nucleotide polymorphism (SNP) marker. SNPs can be assayed and characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or by other biochemical interpretation. SNPs can be sequenced using a variation of the chain termination method (Sanger et al., Proc. Natl. Acad. Sci. (U.S.A.) 74: 5463-5467 (1977)) in which the use of radioisotopes are replaced with fluorescently-labeled dideoxy nucleotides and subjected to capillary based automated sequencing (U.S. Pat. No. 5,332,666, the entirety of which is herein incorporated by reference; U.S. Pat. No. 5,821,058, the entirety of which is herein incorporated by reference). Automated sequencers are available from, for example, Applied Biosystems, Foster City, Calif. (3730xl DNA Analyzer), Beckman Coulter, Fullerton, Calif. (CEQ™ 8000 Genetic Analysis System) and LI-COR, Inc., Lincoln, Nebr. (4300 DNA Analysis System).

The most common marker (e.g., SNP) genotyping methods include hybridization-based (e.g., SNP microarrays), enzyme-based (e.g., primer extension), oligonucleotide ligation, endonuclease cleavage, or a variation of the aforementioned techniques. Primer-extension assays, such as solid-phase minisequencing or pyrosequencing method, a DNA polymerase is used specifically to extend a primer that anneals immediately adjacent to the variant nucleotide. A single labeled nucleoside triphospate complementary to the nucleotide at the variant site is used in the extension reaction. Only those sequences that contain the nucleotide at the variant site will be extended by the polymerase. A primer array can be fixed to a solid support wherein each primer is contained in four small wells, each well being used for one of the four nucleoside triphosphates present in DNA. Template DNA or RNA from each test organism is put into each well and allowed to anneal to the primer. The primer is then extended one nucleotide using a polymerase and a labeled di-deoxy nucleotide triphosphate. The completed reaction can be imaged using devices that are capable of detecting the label which can be radioactive or fluorescent. Using this method several different SNPs can be visualized and detected (Syvänen et al., Hum. Mutat. 13: 1-10 (1999)). The pyrosequencing technique is based on an indirect bioluminometric assay of the pyrophosphate (PPi) that is released from each dNTP upon DNA chain elongation. Following Klenow polymerase mediated base incorporation, PPi is released and used as a substrate, together with adenosine 5-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing procedure (Alderborn et al., Genome Res. 10: 1249-1258 (2000)). An example of an instrument designed to detect and interpret the pyrosequencing reaction is available from Biotage, Charlottesville, Va. (PyroMark MD).

Another marker (e.g., SNP) detection method based on primer-extension assays is a GOOD assay. The GOOD assay (Sauer et al., Nucleic Acids Res. 28: e100 (2000)) is an allele-specific primer extension protocol that employs MALDI-TOF (matrix-assisted laser desorption/ionization time-of-flight) mass spectrometry. The region of DNA containing a SNP is amplified first by PCR amplification. Residual dNTPs are destroyed using an alkaline phosphatase. Allele-specific products are then generated using a specific primer, a conditioned set of a-S-dNTPs and a-S-ddNTPs and a fresh DNA polymerase in a primer extension reaction. Unmodified DNA is removed by 5′ phosphodiesterase digestion and the modified products are alkylated to increase the detection sensitivity in the mass spectrometric analysis. All steps are carried out in a single vial at the lowest practical sample volume and require no purification. The extended reaction can be given a positive or negative charge and is detected using mass spectrometry (Sauer et al., Nucleic Acids Res. 28: e13 (2000)). An instrument in which the GOOD assay is analyzed is for example, the AUTOFLEX® MALDI-TOF system from Bruker Daltonics (Billerica, Mass.).

In one embodiment of the method described herein, genotyping comprises the use of an oligonucleotide probe. The use of an oligonucleotide probe is based on recognition of heteroduplex DNA molecules and includes oligonucleotide hybridization, TAQ-MAN® assays, molecular beacons, electronic dot blot assays and denaturing high-performance liquid chromatography. Oligonucleotide hybridizations can be performed in mass using micro-arrays (Southern, Trends Genet. 12: 110-115 (1996)). TAQ-MAN® assays, or Real Time PCR, detects the accumulation of a specific PCR product by hybridization and cleavage of a double-labeled fluorogenic probe during the amplification reaction. A TAQ-MAN® assay includes four oligonucleotides, two of which serve as PCR primers and generate a PCR product encompassing the polymorphism to be detected. The other two are allele-specific fluorescence-resonance-energy-transfer (FRET) probes. FRET probes incorporate a fluorophore and a quencher molecule in close proximity so that the fluorescence of the fluorophore is quenched. The signal from a FRET probes is generated by degradation of the FRET oligonucleotide, so that the fluorophore is released from proximity to the quencher, and is thus able to emit light when excited at an appropriate wavelength. In the assay, two FRET probes bearing different fluorescent reporter dyes are used, where a unique dye is incorporated into an oligonucleotide that can anneal with high specificity to only one of the two alleles. Useful reporter dyes include 6-carboxy-4,7,2′,7′-tetrachlorofluorecein (TET), 2′-chloro-7′-phenyl-1,4-dichloro-6-carboxyfluorescein (VIC) and 6-carboxyfluorescein phosphoramidite (FAM). A useful quencher is 6-carboxy-N,N,N′,N′-tetramethylrhodamine (TAMRA). Annealed (but not non-annealed) FRET probes are degraded by TAQ DNA polymerase as the enzyme encounters the 5′ end of the annealed probe, thus releasing the fluorophore from proximity to its quencher. Following the PCR reaction, the fluorescence of each of the two fluorescers, as well as that of the passive reference, is determined fluorometrically. The normalized intensity of fluorescence for each of the two dyes will be proportional to the amounts of each allele initially present in the sample, and thus the genotype of the sample can be inferred. An example of an instrument used to detect the fluorescence signal in TAQ-MAN® assays, or Real Time PCR are the 7500 Real-Time PCR System (Applied Biosystems, Foster City, Calif.).

Molecular beacons are oligonucleotide probes that form a stem-and-loop structure and possess an internally quenched fluorophore. When they bind to complementary targets, they undergo a conformational transition that turns on their fluorescence. These probes recognize their targets with higher specificity than linear probes and can easily discriminate targets that differ from one another by a single nucleotide. The loop portion of the molecule serves as a probe sequence that is complementary to a target nucleic acid. The stem is formed by the annealing of the two complementary arm sequences that are on either side of the probe sequence. A fluorescent moiety is attached to the end of one arm and a nonfluorescent quenching moiety is attached to the end of the other arm. The stem hybrid keeps the fluorophore and the quencher so close to each other that the fluorescence does not occur. When the molecular beacon encounters a target sequence, it forms a probe-target hybrid that is stronger and more stable than the stem hybrid. The probe undergoes spontaneous conformational reorganization that forces the arm sequences apart, separating the fluorophore from the quencher, and permitting the fluorophore to fluoresce (Bonnet et al., 1999). The power of molecular beacons lies in their ability to hybridize only to target sequences that are perfectly complementary to the probe sequence, hence permitting detection of single base differences (Kota et al., Plant Mol. Biol. Rep. 17: 363-370 (1999)). Molecular beacon detection can be performed for example, on the Mx4000® Multiplex Quantitative PCR System from Stratagene (La Jolla, Calif.).

In one embodiment, the SNP marker described in the methods provided herein can be identified by a corresponding nucleic acid molecule (e.g., oligonucleotide probe) that comprises at least 15 nucleotides and has at least at least 90% (90%, 91%, 92%, 93%, 94%, 95%, 96%, 97%, 98%, 99%, or 100%) sequence identity to a sequence of the same number of consecutive nucleotides in either sense or antisense strand of DNA that include or are immediately adjacent to the SNP in the soybean genome. For example, the deletion marker disclosed herein is capable of being identified by a corresponding nucleic acid molecule that comprises at least 15 nucleotides that include or are immediately adjacent to the deletion, or by a nucleic acid molecule that only binds to the unique junction formed by the deletion event. In some embodiments, the SNP markers can be detected using a pair of primers, i.e., a first primer and a second primer each comprising at least 15 nucleotides. In some embodiments, the first primer has at least 90% sequence identity to a sequence of the same number of contiguous nucleotides of a sense DNA strand of a region comprising or adjacent to the SNP marker, and the second primer has at least 90% sequence identity to a sequence of the same number of contiguous nucleotides of an antisense DNA strand of the region comprising or adjacent to the SNP marker. In some embodiments, a low-saponin SNP marker is located in a genomic region 132866-141435 of soybean chromosome 7 of the soybean genome. The low-saponin SNP markers can be a T or an A at position 133425 and/or an A or a G at position 136615 of chromosome 7 of the soybean genome, wherein the T at position 133425 or the A at position 136615 of chromosome 7 of the soybean genome is associated with low-saponin content. Accordingly, in some embodiments, the low-saponin SNP markers provided herein are detected using an oligonucleotide probe comprising a nucleic acid sequence having at least 90% identity to a nucleic acid sequence of SEQ ID NOs: 17 and 21 or a nucleic acid sequence of SEQ ID NOs: 17 and 21 (probes for a desirable SNP). The absence of low-saponin SNP markers can be detected using an oligonucleotide probe having at least 90% identity to a nucleic acid sequence of SEQ ID NOs: 18 and 22 or a nucleic acid sequence of SEQ ID NOs: 18 and 22 (probes for an undesirable SNP). The low-saponin SNP markers can also be detected using first and second primers comprising nucleic acid sequences having at least 90% sequence identity to a pair of SEQ ID NOs: 13 and 14, SEQ ID NOs: 15 and 16, or SEQ ID NOs: 19 and 20; or a nucleic acid sequence of NOs: 13 and 14, SEQ ID NOs: 15 and 16, or SEQ ID NOs: 19 and 20.

The electronic dot blot assay uses a semiconductor microchip comprised of an array of microelectrodes covered by an agarose permeation layer containing streptavidin. Biotinylated amplicons are applied to the chip and electrophoresed to selected pads by positive bias direct current, where they remain embedded through interaction with streptavidin in the permeation layer. The DNA at each pad is then hybridized to mixtures of fluorescently labeled allele-specific oligonucleotides. Single base pair mismatched probes can then be preferentially denatured by reversing the charge polarity at individual pads with increasing amperage. The array is imaged using a digital camera and the fluorescence quantified as the amperage is ramped to completion. The fluorescence intensity is then determined by averaging the pixel count values over a region of interest (Gilles et al., Nature Biotech. 17: 365-370 (1999)).

A more recent application based on recognition of heteroduplex DNA molecules uses denaturing high-performance liquid chromatography (DHPLC). This technique represents a highly sensitive and fully automated assay that incorporates a Peltier-cooled 96-well autosampler for high-throughput SNP analysis. It is based on an ion-pair reversed-phase high performance liquid chromoatography method. The heart of the assay is a polystyrene-divinylbenzene copolymer, which functions as a stationary phase. The mobile phase is composed of an ion-pairing agent, triethylammonium acetate (TEAA) buffer, which mediates the binding of DNA to the stationary phase, and an organic agent, acetonitrile (ACN), to achieve subsequent separation of the DNA from the column. A linear gradient of CAN allows the separation of fragments based on the presence of heteroduplexes. DHPLC thus identifies mutations and polymorphisms that cause heteroduplex formation between mismatched nucleotides in double-stranded PCR-amplified DNA. In a typical assay, sequence variation creates a mixed population of heteroduplexes and homoduplexes during reannealing of wild-type and mutant DNA. When this mixed population is analyzed by DHPLC under partially denaturing temperatures, the heteroduplex molecules elute from the column prior to the homoduplex molecules, because of their reduced melting temperatures (Kota et al., Genome 44: 523-528 (2001)). An example of an instrument used to analyze SNPs by DHPLC is the WAVE® HS System from Transgenomic, Inc. (Omaha, Nebr.).

A microarray-based method for high-throughput monitoring of plant gene expression can be utilized as a genetic marker system. This ‘chip′-based approach involves using microarrays of nucleic acid molecules as gene-specific hybridization targets to quantitatively or qualitatively measure expression of plant genes (Schena et al., Science 270:467-470 (1995), the entirety of which is herein incorporated by reference; Shalon, Ph.D. Thesis. Stanford University (1996), the entirety of which is herein incorporated by reference). Every nucleotide in a large sequence can be queried at the same time. Hybridization can be used to efficiently analyze nucleotide sequences. Such microarrays can be probed with any combination of nucleic acid molecules. Particularly preferred combinations of nucleic acid molecules to be used as probes include a population of mRNA molecules from a known tissue type or a known developmental stage or a plant subject to a known stress (environmental or man-made) or any combination thereof (e.g. mRNA made from water stressed leaves at the 2 leaf stage). Expression profiles generated by this method can be utilized as markers.

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5: 874-879 (1989)). Under denaturing conditions, a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis.

In one embodiment of the method described herein, the oligonucleotide probe is adjacent to a polymorphic nucleotide position in the low-saponin QTL. For the purpose of QTL mapping, the markers included must be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes. In one embodiment of the method described herein, genotyping comprises detecting a haplotype.

GEMMA GWAS methods can be used to identify the top genomic regions (QTL) associated with the low-saponin trait.

A maximum likelihood estimate (MLE) for the presence of a marker is calculated, together with an MLE assuming no QTL effect, to avoid false positives. A log10 of an odds ratio (LOD) is then calculated as: LOD=log10 (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), and further described by Arús and Moreno-Gonzalez, Plant Breeding, Hayward, Bosemark, Romagosa (eds.) Chapman & Hall, London, pp. 314-331 (1993).

Additional models can be used for marker and QTL detection. Many modifications and alternative approaches to interval mapping have been reported, including the use of non-parametric methods (Kruglyak and Lander, Genetics, 139:1421-1428 (1995), the entirety of which is herein incorporated by reference). Multiple regression methods or models can also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen and Stam, Genetics, 136:1447-1455 (1994) and Zeng, Genetics, 136:1457-1468 (1994). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng, Genetics, 136:1457-1468 (1994)). These models can be extended to multi-environment experiments to analyze genotype-environment interactions (Jansen et al., Theo. Appl. Genet. 91:33-37 (1995).

Selection of appropriate mapping populations is important to map construction. The choice of an appropriate mapping population depends on the type of marker systems employed (Tanksley et al., Molecular mapping of plant chromosomes. chromosome structure and function: Impact of new concepts J. P. Gustafson and R. Appels (eds.). Plenum Press, New York, pp. 157-173 (1988), the entirety of which is herein incorporated by reference). Consideration must be given to the source of parents (adapted vs. exotic) used in the mapping population. Chromosome pairing and recombination rates can be severely disturbed (suppressed) in wide crosses (adapted × exotic) and generally yield greatly reduced linkage distances. Wide crosses will usually provide segregating populations with a relatively large array of polymorphisms when compared to progeny in a narrow cross (adapted × adapted).

An F2 population is the first generation of selfing after the hybrid seed is produced. Usually a single F1 plant is selfed to generate a population segregating for all the genes in Mendelian (1:2:1) fashion. Maximum genetic information is obtained from a completely classified F2 population using a codominant marker system (Mather, Measurement of Linkage in Heredity: Methuen and Co., (1938), the entirety of which is herein incorporated by reference). In the case of dominant markers, progeny tests (e.g., F3, BCF2) are required to identify the heterozygotes, thus making it equivalent to a completely classified F2 population. However, this procedure is often prohibitive because of the cost and time involved in progeny testing. Progeny testing of F2 individuals is often used in map construction where phenotypes do not consistently reflect genotype (e.g. disease resistance) or where trait expression is controlled by a QTL. Segregation data from progeny test populations (e.g. F3 or BCF2) can be used in map construction. Marker-assisted selection can then be applied to cross progeny based on marker-trait map associations (F2, F3), where linkage groups have not been completely disassociated by recombination events (i.e., maximum disequilibrium).

In certain embodiments, additional markers linked to a low-saponin allele. This may be carried out, for example, by first preparing an F2 population by selfing an F1 hybrid produced by crossing inbred varieties only one of which comprises a low-saponin allele. Recombinant inbred lines (RIL) (genetically related lines, usually F5 or progeny thereof, developed from continuously selfing F2 lines towards homozygosity) can then be prepared and used as a mapping population. Information obtained from dominant markers can be maximized by using RIL because all or nearly loci are homozygous. The genetic linkage of additional marker molecules can be established by a gene mapping model such as, without limitation, the flanking marker model reported by Lander and Botstein, Genetics, 121:185-199 (1989), and the interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y., the manual of which is herein incorporated by reference in its entirety). Use of Qgene software is a particularly preferred approach.

Backcross populations (e.g., generated from a cross between a desirable variety (recurrent parent) and another variety (donor parent) carrying a trait not present in the former can also be utilized as a mapping population. A series of backcrosses to the recurrent parent can be made to recover most of its desirable traits. Thus a population is created consisting of individuals similar to the recurrent parent but each individual carries varying amounts of genomic regions from the donor parent. Backcross populations can be useful for mapping dominant markers if all loci in the recurrent parent are homozygous and the donor and recurrent parent have contrasting polymorphic marker alleles (Reiter et al., 1992).

Useful populations for mapping purposes are near-isogenic lines (NIL). NILs are created by many backcrosses to produce an array of individuals that are nearly identical in genetic composition except for the desired trait or genomic region can be used as a mapping population. In mapping with NILs, only a portion of the polymorphic loci are expected to map to a selected region. Mapping may also be carried out on transformed plant lines.

In one embodiment, the method further comprises determining the saponin content of the second population of soybean plants or seeds, wherein the second population of soybean plants or seeds is progeny soybean plants or seeds produced from the first population of soybean plants or seeds comprising one or more alleles comprising one or more low-saponin QTLs. The low-saponin QTL can be one or more of Gm07_137242, Gm07_133425, and Gm07_136615. Saponin content in a plant, plant part, or plant product can be measured by any standard methods of measuring or estimating the amount of saponins in a sample. For example, saponin content can be determined by using colorimetry, high performance liquid chromatography (HPLC), mass spectrometry, or liquid chromatography and tandem mass spectrometry (e.g., LC-MS/MS).

### D. Nucleic Acid Molecules for Detecting a Molecular Marker in Soybean Genome

Provided herein is a nucleic acid molecule for detecting a low-saponin molecular marker in soybean DNA. In some embodiments, the nucleic acid molecule is an oligonucleotide probe. In some embodiments, the nucleic acid molecule comprises at least 15 nucleotides and has at least 90% (91%, 92%, 93%, 94%, 95%, 96%, 97%, 98%, 99%, or 100%) sequence identity to a sequence of the same number of consecutive nucleotides in a sense or antisense strand of DNA in a region comprising or adjacent (e.g., immediately adjacent) to the molecular marker. In some embodiments, the low-saponin molecular marker is located in a genomic region 132866-141435 of chromosome 7 of the soybean genome. In some embodiments, the molecular marker is an SNP marker. Example low-saponin SNP markers include a T or an A at position 133425 and/or an A or a G at position 136615 of chromosome 7 of the soybean genome, wherein the T at position 133425 or the A at position 136615 of chromosome 7 of the soybean genome is associated with low-saponin content. In some embodiments, the nucleic acid molecule (e.g., an oligonucleotide probe) described herein comprises SEQ ID NOs: 17 and 21 for detection of a desirable low-saponin marker, and SEQ ID NOs: 18 and 22 for detection of the absence of a desirable low-saponin marker. The nucleic acid molecule can comprise a nucleic acid sequence having at least 90% sequence identity to any one of SEQ ID NOs: 17, 18, 21, and 22. The nucleic acid molecule can further comprise a detectable label, e.g., a fluorescent label (quencher) (e.g., MGB), or a radioactive label.

Also provided herein is a pair of nucleic acid molecules (e.g., a pair of primers) for detecting a low-saponin molecular marker by primer extension method, e.g., PCR. The pair of nucleic acid molecules can comprise a first primer and a second primer each comprising at least 15 nucleotides, with the first primer having at least 90% sequence identity to a sequence of the same number of contiguous nucleotides of a sense DNA strand of a region comprising or adjacent to the molecular marker, and the second primer having at least 90% sequence identity to a sequence of the same number of contiguous nucleotides of an antisense DNA strand of the region comprising or adjacent to the molecular marker. In some embodiments, the low-saponin molecular marker is located in a genomic region 132866-141435 of chromosome 7. The pair of primers can be used to detect the presence or absence of a low-saponin SNP marker, e.g., a T or an A at position 133425 and/or an A or a G at position 136615 of chromosome 7 of the soybean genome, or the presence or absence of a deletion marker, e.g., a deletion of positions Gm07_137242-137246 of the soybean genome. In some embodiments, the first and second primers comprise nucleic acid sequences having at least 90% identity to any one pair of SEQ ID NOs: 13 and 14; SEQ ID NOs: 15 and 16, SEQ ID NOs: 19 and 20, or a nucleic acid sequence of SEQ ID NOs: 13 and 14; SEQ ID NOs: 15 and 16, SEQ ID NOs: 19 and 20.

### E. Breeding of Soybean Plants Comprising Low Saponin Content

Low-saponin soybean plants of the present disclosure can be part of or generated from a breeding program. The choice of breeding method depends on the mode of plant reproduction, the heritability of the trait(s) being improved, and the type of cultivar used commercially (e.g., F1 hybrid cultivar, pureline cultivar, etc.). A cultivar is a race or variety of a plant that has been created or selected intentionally and maintained through cultivation.

Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

Selected, non-limiting approaches for breeding the plants of the present invention are set forth below. A breeding program can be enhanced using marker assisted selection (MAS) of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination event, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new soybean cultivars requires the development and selection of soybean varieties, the crossing of these varieties and selection of superior hybrid crosses. The hybrid seed can be produced by manual crosses between selected male-fertile parents or by using male sterility systems. Hybrids are selected for certain single gene traits such as pod color, flower color, seed yield, pubescence color or herbicide resistance which indicate that the seed is truly a hybrid. Additional data on parental lines, as well as the phenotype of the hybrid, influence the breeder’s decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits (e.g., low saponin) are crossed to produce an F1. An F2 population is produced by selfing one or several F1′s. Selection of the best individuals in the best families is selected. Replicated testing of families can begin in the F4 generation to improve the effectiveness of selection for traits with low heritability. At an advanced stage of inbreeding (i.e., F6 and F7), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

In a multiple-seed procedure, soybean breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seed of a population each generation of inbreeding.

Descriptions of other breeding methods that are commonly used for different traits and crops can be found in one of several reference books (e.g., Fehr, Principles of Cultivar Development Vol. 1, pp. 2-3 (1987)).

### F. Plants, Plant Parts, and Plant Products Produced by Present Methods

Provided herein is a soybean plant or soybean seed selected, generated, or produced by any methods disclosed herein and having low saponin content. In some embodiments, such low saponin soybean plant or seed comprises one or more low-saponin QTLs. A low-saponin QTL of the soybean plant or soybean seed can be located within a genomic region 132866-141435 of chromosome 7 of a soybean genome, such as Gm07_137242, Gm07_133425, and/or Gm07_136615. A low-saponin SNP marker can be a T or an A at position 133425 and/or an A or a G at position 136615 of chromosome 7 of the soybean genome, wherein the T at position 133425 or the A at position 136615 of chromosome 7 of the soybean genome is associated with low-saponin content. A low-saponin deletion marker can be a deletion of positions Gm07_137242-137246 of the soybean genome.

Also provided herein is a population of low-saponin soybean plants or soybean seeds selected, generated, or produced by any methods disclosed herein and having low saponin content. In some embodiments, such population of low-saponin soybean plants or seeds comprises one or more low-saponin QTLs at a greater frequency relative to a control population of soybean plants or seeds not having low-saponin content.

Also provided herein is a population of soybean plants or soybean seeds comprising at least one low-saponin QTL provided herein at a greater frequency than a control population of soybean plants or seeds. Such population of soybean plants or seeds can have lower saponin content relative to a control population of soybean plants or seeds having the low-saponin QTL at less frequency.

In some embodiments, a control population of soybean plants or seeds is a population produced by methods without assaying for or selecting based on a low-saponin molecular marker disclosed herein. A population of low-saponin soybean plants and seeds of the present disclosure can include soybean plants and seeds that contain a low-saponin molecular marker disclosed herein, as well as soybean plants and seeds that do not contain a low-saponin molecular marker disclosed herein. The low-saponin soybean plants and seeds of the present disclosure can be produced, exclusively or nonexclusively, from plants or seeds that contain a low-saponin molecular marker disclosed herein, or can be produced, exclusively or nonexclusively, from plants or seeds that do not contain a low-saponin molecular marker disclosed herein.

Also provided herein are soybean plant parts (e.g., seed, juice, pulp, fruit, flowers, nectar, embryos, pollen, ovules, leaves, stems, branches, kernels, stalks, roots, root tips, anthers, etc.) and plant products produced from soybean plants or seeds of the present disclosure. A “plant product”, as used herein, refers to any product or composition produced from the plant or plant part, including oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass). A plant product of the present disclosure is discussed further hereinabove. The plant parts and plant products provided herein can comprise low saponin content, one or more low-saponin molecular markers, and other characteristics (e.g., decreased BAS activity) as provided elsewhere herein. For example, plants, plant parts, or plant products produced by the present methods can have total saponin content or DDMP saponin content that is lower by at least 5%, 10%, 15%, 20%, 25%, 30%, 35%, 40%, 45%, 50%, 55%, 60%, 65%, 70%, 75%, 80%, 85%, 90%, 95%, or 99%; or about at least 5%, 10%, 15%, 20%, 25%, 30%, 35%, 40%, 45%, 50%, 55%, 60%, 65%, 70%, 75%, 80%, 85%, 90%, 95%, 99%, or 100%; or about 5-100%, 10-100%, 20-100%, 30-100%, 40-100%, 50-100%, 60-100%, 70-100%, 75-100%, 75-80%, 80-100%, 90-100%, or 97-100% as compared to a control plant, plant part, or plant product (e.g., not comprising the low-saponin marker). Plants, plant parts, or plant products provided herein can contain total saponin content that is lower than a control plant, plant part, or plant product, such as less than about 2.7-7.0 mg/g (e.g., 6.5 mg/g or less, 6.0 mg/g or less, 5.5 mg/g or less, 5.0 mg/g or less, 4.5 mg/g or less, 4.0 mg/g or less, 3.5 mg/g or less, 3.0 mg/g or less, 2.7 mg/g or less, 2.5 mg/g or less, 2.0 mg/g or less, 1.5 mg/g or less, 1.0 mg/g or less, 0.5 mg/g or less), and/or DDMP saponin content of less than about 2.0-5.5 mg/g (e.g., 5.5 mg/g or less, 5.0 mg/g or less, 4.5 mg/g or less, 4.0 mg/g or less, 3.5 mg/g or less, 3.0 mg/g or less, 2.5 mg/g or less, 2.0 mg/g or less, 1.5 mg/g or less, 1.0 mg/g or less, 0.5 mg/g or less). In specific embodiments, a plant, plant part, or plant product produced by the methods provided herein comprises contains from about 0 mg/g to about 0.8 mg/g of total saponins, and/or from about 0 mg/g to about 0.6 mg/g of DDMP saponins.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting. Unless otherwise noted, all parts and percentages are by dry weight.

### EXAMPLES

### Example 1: Expression of Beta-Amyrin Synthase (BAS) Copies in Wild-Type Soybean Tissues

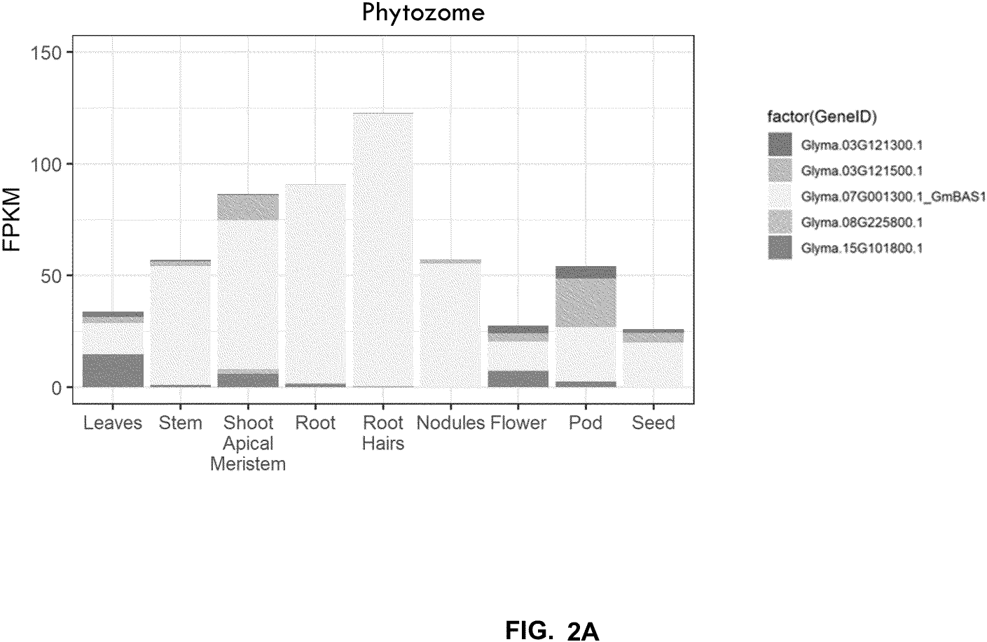

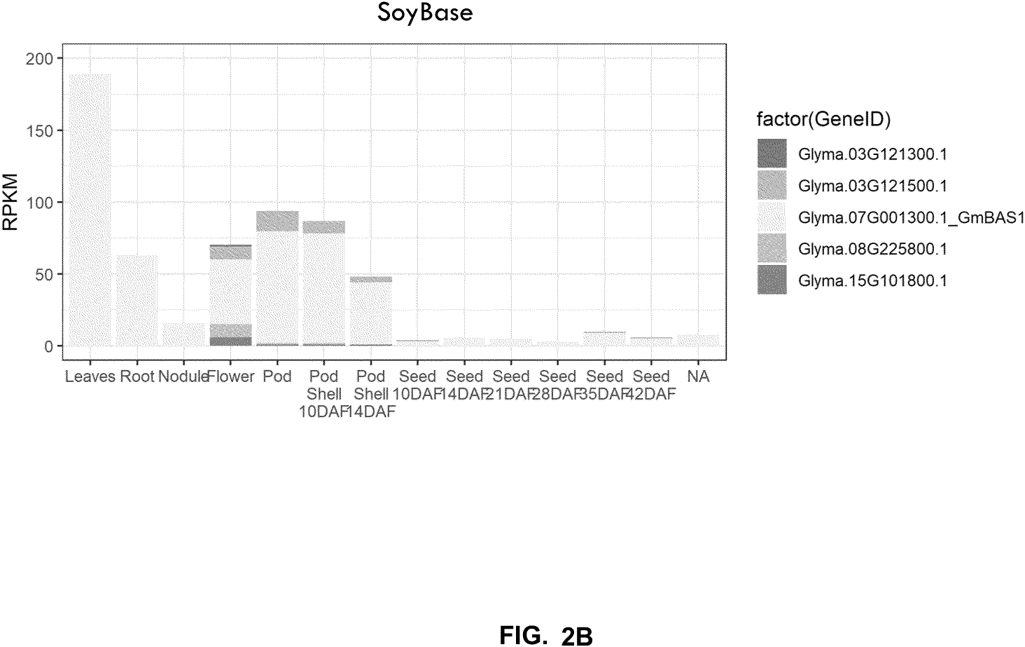

Transcript expression levels of five BAS copies in soybean, Glycine max beta-amyrin synthase gene 1 (GmBAS1, Glyma.07g001300), Glycine max beta-amyrin synthase gene 2 (GmBAS2, Glyma.08g225800), Glycine max beta-amyrin synthase gene 3 (GmBAS3, Glyma.03g121300), Glycine max beta-amyrin synthase gene 4 (GmBAS4, Glyma.03g121500), and Glycine max beta-amyrin synthase gene 5 (GmBAS5, Glyma.15g101800) were analyzed using the Phytozome and SoyBase databases. As shown in FIGS. 2A and 2B, BAS gene transcripts are expressed across various tissues of soybean, including leaves, stem, shoot, root, nodules, flower, pod and seed. In particular, GmBAS1 is highly expressed across various soybean tissues, and is the highest expressed BAS gene copy in seed tissues. FPKM and RPKM stand for fragments per kilobase of exon per million reads and reads per kilobase million, respectively.

The five copies of soybean BAS shared high sequence similarity to one another, as shown in Table 2.

TABLE 2

Pairwise coding sequence similarity between BAS 1 and the other BAS copies

| | Glyma.08g225800 (GmBAS2) | Glyma.03g121300 (GmBAS3) | Glyma.03g121500 (GmBAS4) | Glyma.15g101800 (GmBAS5) |
|---|---|---|---|---|
| Glyma.07g001300 (GmBAS1) | 93.4% | 89.8% | 73.7% | 73.6% |

### Example 2: Gene Editing of the BAS Gene

GmBAS1 (Glyma.07g001300) comprises 16 exons. Guide RNAs targeting GmBAS1 were designed according to standard methods of the art (Zetsche et al., Cell, Volume 163, Issue 3, Pages 759-771, 2015; Cui et al., Interdisciplinary Sciences: Computational Life Sciences, volume 10, pages 455-465, 2018). Optimized gRNA design to maximize activity and minimize off-target effects of CRISPR-Cas9 and CRISPR-Cas12a have been extensively characterized (Nat Biotechnol 34, 184-191, doi: 10.1038/nbt.3437 (2016)). The CRISPR-Cas12a system described herein can be employed for targeting PAM sites such as TTN, TTV, TTTV, NTTV, TATV, TATG, TATA, YTTN, GTTA, and GTTC, utilizing corresponding gRNAs.

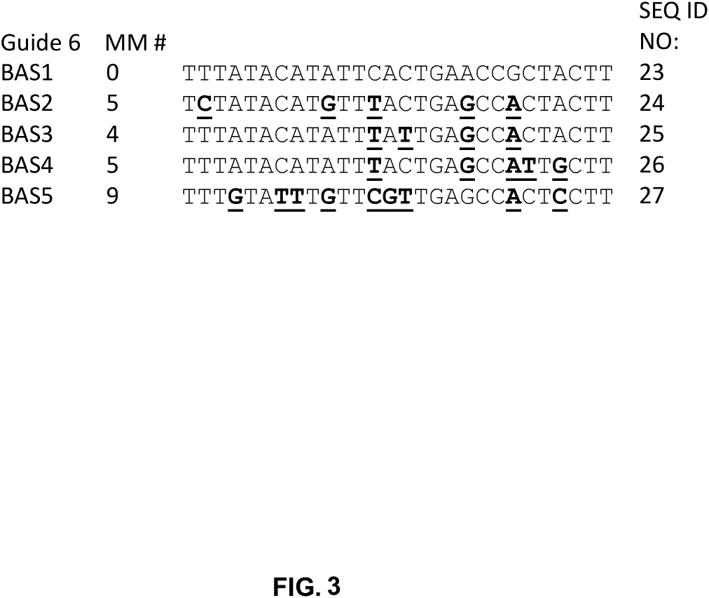

As shown in FIG. 3, GmBAS1 guide RNA 6, which targets a nucleic acid region in exon 7, is complementary to the nucleic acid sequence of GmBAS1 (without mismatched base), and has sequence specificity to GmBAS1 over other copies of soybean BAS, GmBAS2-5. The nucleic acid sequences encoding targeting sequence of GmBAS1 guide RNA 6 is set forth as SEQ ID NO: 12. GmBAS1 guide RNA 6 showed at least approximately 1% editing efficiency in soybean protoplasts with Agrobacterium transformation.

Embryonic axes of mature seeds of soybean varieties were transformed with constructs comprising GmBAS1 guide RNA6 and a nuclease using Agrobacterium transformation. Transformed plants were identified by their resistance to spectinomycin. Amplicons were produced of the genomic regions near the targeted GmBAS1 site and sequenced to evaluate the presence of the mutation by using forward primer GATAGTCGTTCATTATGTCAATC (SEQ ID NO: 13) and reverse primer CACACAACCAATGGTTATG (SEQ ID NO: 14). Transgenic events were recorded, and the T0 plants were assigned unique plant names (e.g., Plant A) and were subjected to molecular characterization and propagation.

T0 plants were self-pollinated and T1 plants were generated. As shown in Table 3, of the 74 total T1 plants, 57 plants (114 potential alleles) carried mutations and 27 new fixed alleles were identified. A “fixed” allele, as used herein, refers to an allele having a consistent mutation (e.g., insertion-deletion) profile across proliferating tissue in a T1 plant.

TABLE 3

Characteristics of T1 plants

| Target | BAS1 |
|---|---|
| Total T1 Plants | 74 |
| NGS Plants | 57 |
| Potential Alleles | 114 |
| New Fixed Alleles | 27 |

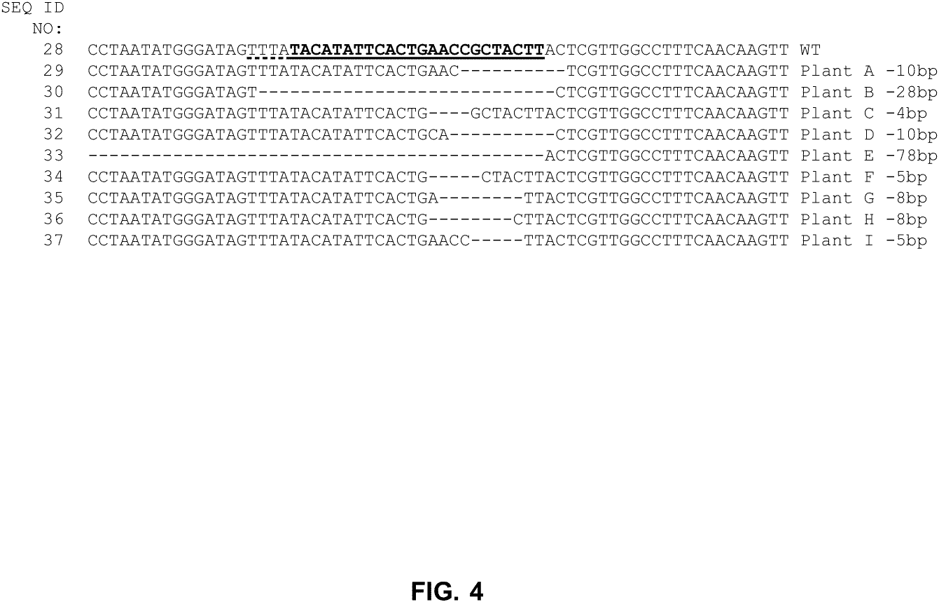

FIG. 4 shows partial nucleic acid sequences of the T1 plants with a mutation around the targeting site of guide RNA6 in exon 7 of GmBAS1. The underlined sequence in the WT plant sequence shows the targeting sequence of guide RNA6. In an exemplary plant P178967.1:66, a homozygous deletion of 5 bp was identified.

Transformed plants are screened using a variety of molecular tools to identify plants and genotypes that will result in the expected phenotype. T2 seed was harvested from select T1 plants that were homozygous for the edit and null for the T-DNA insertion.

### Example 3: Screening of Plants With Mutations

To determine if mutations generated in the BAS gene or a regulatory region of the BAS gene result in decreased expression levels of BAS gene or BAS protein and/or decreased BAS activity in the plants, homozygous mutant plants are generated. In the mutant plants, activity of beta-amyrin synthase is measured by one or more standard methods of measuring enzyme activity, e.g., enzyme assays. For example, BAS activity in the plant (e.g., function or activity of BAS protein in the plant) is determined by contacting a substrate (e.g., 2, 3-oxidosqualene) with a sample obtained from a plant, plant part, or plant product and measuring the level of the product, e.g., beta-amyrin, e.g., by gas chromatography-mass spectrometry (GC-MS). Expression levels of the BAS gene are measured by any standard methods for measuring mRNA levels of a gene, including quantitative RT-PCR, northern blot, and serial analysis of gene expression (SAGE). Expression levels of the BAS gene or BAS protein (e.g., full-length BAS protein) are measured by any standard methods for measuring protein levels, including western blot analysis, ELISA, or dot blot analysis of a protein sample obtained from the plant using an antibody directed to the BAS protein (e.g., full-length BAS protein). Function or activity of a BAS protein in a plant, plant part, or plant product is determined by one or more standard methods for measuring enzyme activity, e.g., enzyme assays. The plant with mutation may have decreased BAS activity (e.g., decreased function or activity of the BAS protein), decreased expression levels of the BAS gene or the BAS protein, or decreased beta-amyrin levels as compared to a control plant (e.g., without the mutation) when grown under the same environmental conditions.

To determine if mutations generated in the BAS gene result in decreased saponin production, homozygous mutant plants were generated. In the mutant soybean plant seeds, the content of total saponins and/or DDMP saponins (a particularly astringent saponin) was measured by high performance liquid chromatography (HPLC). The plant with mutation may have decreased saponin levels as compared to a control plant (e.g., without the mutation) when grown under the same environmental conditions.

### Example 4: Chemical Mutagenesis in the BAS Gene

Soybean seeds were treated with 0.3% ethyl methanesulfonate (EMS) and 0.1 mM N-ethyl-N-nitrosourea (ENU) for 16 hours, followed by five washes in pure water. The resulting M0 seeds were planted and grown to maturity. DNA samples were prepared from the M0 plants and sequenced. M0 plants having a mutation in the BAS gene were selected based on the sequencing results.

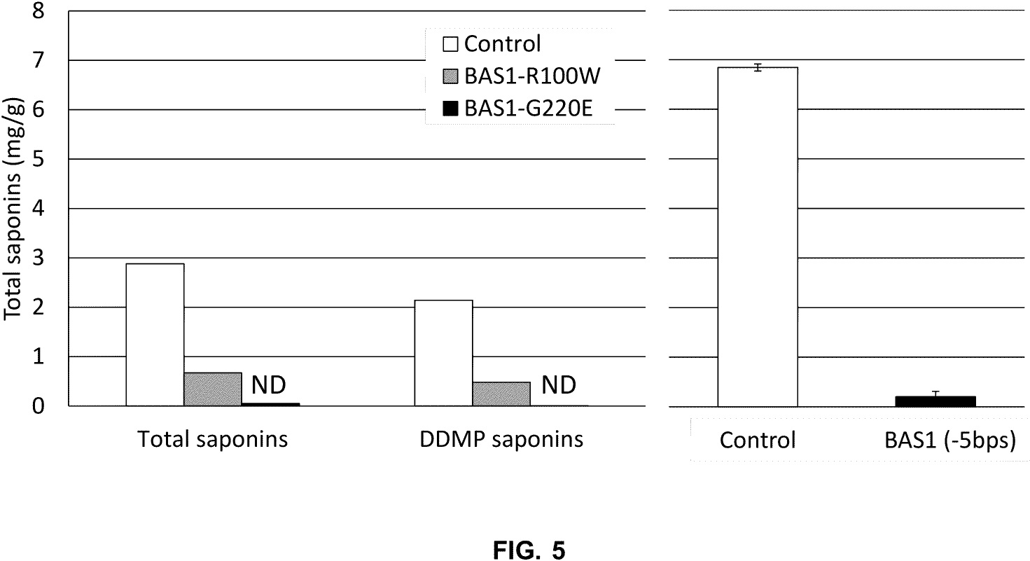

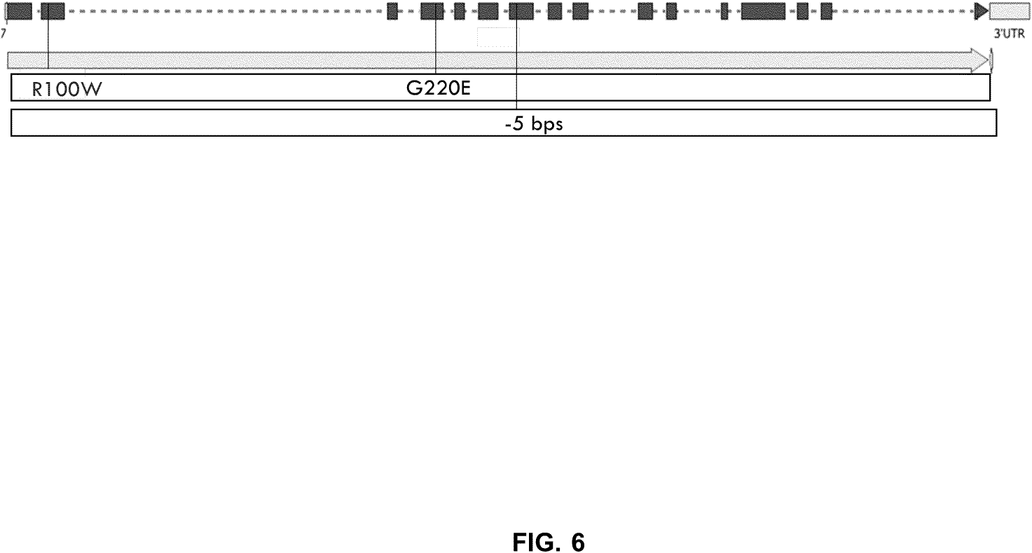

M1 seeds were harvested from M0 plants identified as having a mutation in the BAS gene, and phenotypic analysis were conducted as described in Example 3. As part of the analysis, the content of total saponins and/or DDMP saponins (a particularly astringent saponin) was measured by high performance liquid chromatography (HPLC). As shown in FIG. 5, the total saponins (left panel) and DDMP saponins (middle panel) were decreased by more than 97% in the GmBAS1 G220E mutant seeds (total saponin decreased from 2.88 mg/g to 0.05 mg/g, DDMP saponin decreased from 2.14 mg/g to 0.004 mg/g), and partially reduced in the GmBAS1 R100W mutant seeds (total saponin decreased from 2.88 mg/g to 0.67 mg/g, DDMP saponin decreased from 2.14 mg/g to 0.48 mg/g), relative to control seeds without mutation. The total saponins were reduced by more than 97% (from in the seeds of the soybean plant having a 5 bp deletion in the GmBAS1 gene (at nucleotides 4191-4195 of SEQ ID NO: 1, Plant I; decreased from 6.84 mg/g to 0.20 mg/g) relative to seeds of a control plant without mutation (right panel). As shown in FIG. 6, the R100W, G220E, and -5 bp mutations in these plants and seeds are located in exon 2, exon 4, and exon 7 of the GmBAS1 gene, respectively.

### Example 5: Sensory Results of Plant Protein Isolates

A plant protein isolate is prepared from the grains of the plants with mutation using the standard methods for protein isolation (e.g., acid precipitation method as described in U.S. Pat. Publication No.: US20190191735; incorporated by reference herein). A patty like product is prepared using the plant isolate. Textural characteristics of the patty like products prepared from plants or plant parts with altered BAS activity and/or saponin content (e.g., with mutation in at least one BAS gene and/or a regulatory region of the BAS gene) are evaluated by a panel of trained sensory experts. Patties are formed and evaluated in uncooked and cooked state, and compared to patties obtained from control (e.g., wild-type) plants. The samples are evaluated using a scorecard for a variety of attributes (e.g., bitterness, astringency, beaniness, grassiness, staleness, taste, surface color, browning, aroma, smell, surface texture, oil content, hardness/firmness, chewiness, bite force, mouthfeel, degradation, fattiness, adhesiveness, elasticity, rubberiness, surface thickness, moldability, binding/integrity, grittiness, graininess, lumpiness, greasiness, moistness, sliminess) and quality factors (e.g., flavor, appearance, and texture).

The patty like product prepared from plants or plant parts with altered BAS activity and/or saponin content (e.g., with mutation in at least one BAS gene and/or a regulatory region of the BAS gene) can have superior sensory characteristics (e.g., less bitterness, less astringency, less beaniness, less grassiness, less staleness) compared to a patty like product prepared from a control plant or plant part. Plant protein compositions prepared from plants or plant parts of the present disclosure, e.g., with altered BAS activity and/or saponin content (e.g., with mutation in at least one BAS gene and/or a regulatory region of the BAS gene) can have superior qualities with respect to sensory properties compared to a patty like product prepared from a control plant or plant part.

The section headings used herein are for organizational purposes only and are not to be construed as limiting the subject matter described in any way.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

While various aspects of the invention are described herein, it is not intended that the invention be limited by any particular aspect. On the contrary, the invention encompasses various alternatives, modifications, and equivalents, as will be appreciated by those of skill in the art. Furthermore, where feasible, any of the aspects disclosed herein may be combined with each other (e.g., the feature according to one aspect may be added to the features of another aspect or replace an equivalent feature of another aspect) or with features that are well known in the art, unless indicated otherwise by context.

TABLE 4

Sequence Descriptions

| SEQ ID NO: | DESCRIPTION |
|---|---|
| 1 | Full-length coding sequence of Glycine max beta-amyrin synthase gene 1 (GmBAS1, Glyma.07g001300) |
| 2 | Full-length coding sequence of Glycine max beta-amyrin synthase gene 2 (GmBAS2, Glyma.08g225800) |
| 3 | Coding sequence of Glycine max beta-amyrin synthase gene 3 (GmBAS3, Glyma.03g121300) |
| 4 | Coding sequence of Glycine max beta-amyrin synthase gene 4 (GmBAS4, Glyma.03g121500) |
| 5 | Coding sequence of Glycine max beta-amyrin synthase gene 5 (GmBAS5, Glyma.15g101800) |
| 6 | Amino acid sequence of full-length Glycine max beta-amyrin synthase 1 (GmBAS1) |
| 7 | Amino acid sequence of full-length Glycine max beta-amyrin synthase 2 (GmBAS2) |
| 8 | Amino acid sequence of full-length Glycine max beta-amyrin synthase 3 (GmBAS3) |
| 9 | Amino acid sequence of full-length Glycine max beta-amyrin synthase 4 (GmBAS4) |
| 10 | Amino acid sequence of full-length Glycine max beta-amyrin synthase 5 (GmBAS5) |
| 11 | Nucleic acid sequence of exon 7 of GmBAS1 gene |
| 12 | Nucleic acid sequence encoding targeting sequence of GmBAS1 guide RNA 6 |
| 13 | Forward primer for detecting mutation in GmBAS1 near binding site of GmBAS1 guide RNA 6 |
| 14 | Reverse primer for detecting mutation in GmBAS1 near binding site of GmBAS1 guide RNA 6 |
| 15 | Forward primer for detecting mutation in GmBAS1 at or near nucleotide 374 of SEQ ID NO: 1 (chr07:133425) |
| 16 | Reverse primer for detecting mutation in GmBAS1 at or near nucleotide 374 of SEQ ID NO: 1 (chr07:133425) |
| 17 | Probe sequence for detecting A to T substitution at nucleotide 374 of SEQ ID NO: 1 in GmBAS1 (chr07:133425) |
| 18 | Probe sequence for detecting wild-type sequence (A) at nucleotide 374 of SEQ ID NO: 1 in GmBAS1 (chr07:133425) |
| 19 | Forward primer for detecting mutation in GmBAS1 at or near nucleotide 3564 of SEQ ID NO: 1 (chr07:136615) |
| 20 | Reverse primer for detecting mutation in GmBAS1 at or near nucleotide 3564 of SEQ ID NO: 1 (chr07:136615) |
| 21 | Probe sequence for detecting G to A substitution at nucleotide 3564 of SEQ ID NO: 1 in GmBAS1 (chr07:136615) |
| 22 | Probe sequence for detecting wild-type sequence (G) at nucleotide 3564 of SEQ ID NO: 1 in GmBAS1 (chr07:136615) |
| 23 | DNA fragment from GmBAS1 |
| 24 | DNA fragment from GmBAS2 |
| 25 | DNA fragment from GmBAS3 |
| 26 | DNA fragment from GmBAS4 |
| 27 | DNA fragment from GmBAS5 |
| 28 | DNA fragment from GmBAS1 in wild-type plant |
| 29 | DNA fragment from GmBAS1 in Plant A |
| 30 | DNA fragment from GmBAS1 in Plant B |
| 31 | DNA fragment from GmBAS1 in Plant C |
| 32 | DNA fragment from GmBAS1 in Plant D |
| 33 | DNA fragment from GmBAS1 in Plant E |
| 34 | DNA fragment from GmBAS1 in Plant F |
| 35 | DNA fragment from GmBAS1 in Plant G |
| 36 | DNA fragment from GmBAS1 in Plant H |
| 37 | DNA fragment from GmBAS1 in Plant I |
| 38 | Nucleotide sequence of full-length Glycine max beta-amyrin synthase gene 1 (GmBAS1, Glyma.07g001300) |

What is claimed is:

1. A plant or plant part comprising decreased beta-amyrin synthase (BAS) activity compared to a control plant or plant part, wherein said plant or plant part comprises a genetic mutation that decreases the BAS activity.

2. The plant or plant part of claim 1, comprising decreased saponin content or improved flavor characteristics compared to a control plant or plant part.

3. (canceled)

4. The plant or plant part of claim 1, wherein the mutation comprises one or more insertions, substitutions, or deletions in at least one BAS gene or homolog thereof or regulatory region thereof in said plant or plant part, wherein:
an expression level of said at least one BAS gene or homolog thereof is reduced compared to corresponding at least one BAS gene or homolog thereof without said mutation; or
level or activity of a BAS protein encoded by said at least one BAS gene or homolog thereof is reduced compared to a BAS protein encoded by corresponding at least one native BAS gene or homolog thereof without said mutation.

5. The plant or plant part of claim 4:
(a) wherein the mutation is located in a BAS gene or homolog thereof:
(i) comprising a nucleic acid sequence having at least 80% sequence identity to a nucleic acid sequence of any one of SEQ ID NOs: 1-5 and 38, wherein said nucleic acid sequence encodes a polypeptide that retains BAS activity;
(ii) comprising the nucleic acid sequence of any one of SEQ ID NOs: 1-5 and 38;
(iii) encoding a polypeptide comprising an amino acid sequence having at least 80% sequence identity to an amino acid sequence of any one of SEQ ID NOs: 6-10, wherein said polypeptide retains BAS activity;
(iv) encoding a polypeptide comprising an amino acid sequence of any one of SEQ ID NOs: 6-10; and/or
in a regulatory region of said BAS gene or homolog thereof; or
(b) wherein the mutation is located at least partially in a nucleic acid region of exon 2, 4, or 7 of a Glycine max BAS1 gene; or
(c) wherein the plant or plant part comprises a deletion of about 4-78 nucleotides at least partially in the nucleic acid region of exon 7 of the Glycine max BAS1 gene, a substitution in the nucleic acid region of exon 4 of the Glycine max BAS1 gene, or a substitution in the nucleic acid region of exon 2 of the Glycine max BAS1 gene; or
(d) wherein the plant or plant part comprises: (i) a mutated Glycine max BAS1 gene comprising a deletion of nucleotides 4191 through 4195 of SEQ ID NO: 1; (ii) a mutated Glycine max BAS1 gene comprising a G to A substitution of nucleotide 3564 of SEQ ID NO: 1 or a G to A substitution of nucleotide 3750 of SEQ ID NO_{:} 38; (iii) a mutated Glycine max BAS1 gene comprising an A to T substitution of nucleotide 374 of SEQ ID NO: 1 or an A to T substitution of nucleotide 560 of SEQ ID NO: 38; (iv) a mutated Glycine max BAS1 protein comprising a G to E substitution of amino acid 220 of SEQ ID NO: 6; (v) a mutated Glycine max BAS1 protein comprising an R to W substitution of amino acid 100 of SEQ ID NO: 6; (vi) a mutated Glycine max BAS1 gene comprising a deletion of nucleotides 4190 through 4199 of SEQ ID NO: 1; (vii) a mutated Glycine max BAS1 gene comprising a deletion of nucleotides 4171 through 4198 of SEQ ID NO: 1; (viii) a mutated Glycine max BAS1 gene comprising a deletion of nucleotides 4187 through 4190 of SEQ ID NO: 1; (ix) a mutated Glycine max BAS1 gene comprising a deletion of nucleotides 4189 through 4198 of SEQ ID NO_{:} 1; (x) a mutated Glycine max BAS1 gene comprising a deletion of nucleotides 4120 through 4197 of SEQ ID NO: 1; (xi) a mutated Glycine max BAS1 gene comprising a deletion of nucleotides 4187 through 4191 of SEQ ID NO: 1; (xii) a mutated Glycine max BAS1 gene comprising a deletion of nucleotides 4188 through 4195 of SEQ ID NO: 1; or (xiii) a mutated Glycine max BAS1 gene comprising a deletion of nucleotides 4187 through 4194 of SEQ ID NO:1.

6-18. (canceled)

19. A population of plants or plant parts comprising the plant or plant part of claim 1, wherein the population comprises decreased beta-amyrin synthase (BAS) activity, a decreased saponin content, and/or improved flavor characteristics compared to a control population.

20-21. (canceled)

22. A method for decreasing saponin content in a plant or plant part, said method comprising introducing a genetic mutation that decreases beta-amyrin synthase (BAS) activity into said plant or plant part, wherein BAS activity is decreased and saponin content is decreased in said plant or plant part relative to a control plant or plant part.

23. (canceled)

24. The method of claim 22, wherein the mutation comprises one or more insertions, substitutions, or deletions introduced in at least one BAS gene or homolog thereof or in a regulatory region thereof in said plant or plant part, wherein:
an expression level of said at least one BAS gene or homolog thereof is reduced by said mutation, and/or
level or activity of a BAS protein encoded by said at least one BAS gene or homolog thereof is reduced by said mutation.

25. The method of claim 24:
(a) wherein the mutation is introduced at least partially into a BAS gene or homolog thereof:
(i) comprising a nucleic acid sequence having at least 80% sequence identity to a nucleic acid sequence of any one of SEQ ID NOs: 1-5 and 38, wherein said nucleic acid sequence encodes a polypeptide that retains BAS activity;
(ii) comprising the nucleic acid sequence of any one of SEQ ID NOs: 1-5 and 38;
(iii) encoding a polypeptide comprising an amino acid sequence having at least 80% sequence identity to an amino acid sequence of any one of SEQ ID NOs: 6-10, wherein said polypeptide retains BAS activity; and/or
(iv) encoding a polypeptide comprising an amino acid sequence of any one of SEQ ID NOs: 6-10,
or into a regulatory region of said BAS gene or homolog thereof, or
(b) wherein the mutation is introduced to locate at least partially in a nucleic acid region of exon 2, 4, or 7 of a Glycine max BAS1 gene, or
(c) wherein said mutation comprises a deletion of about 4-78 nucleotides at least partially in the nucleic acid region of exon 7 of the Glycine max BAS1 gene, a substitution in the nucleic acid region of exon 4 of the Glycine max BAS1 gene, or a substitution in the nucleic acid region of exon 2 of the Glycine max BAS1 gene; or
(d) wherein (i) the mutation comprises a deletion of nucleotides 4191 through 4195 of SEQ ID NO: 1; (ii) the mutation comprises a G to A substitution of nucleotide 3564 of SEQ ID NO: 1 or a G to A substitution of nucleotide 3750 of SEQ ID NO: 38; (iii) the mutation comprises an A to T substitution of nucleotide 374 of SEQ ID NO: 1 or an A to T substitution of nucleotide 560 of SEQ ID NO: 38; (iv) the mutation produces a G to E substitution of amino acid 220 of SEQ ID NO: 6; (v) the mutation produces an R to W substitution of amino acid 100 of SEQ ID NO: 6; (vi) the mutation comprises a deletion of nucleotides 4190 through 4199 of SEQ ID NO: 1; (vii) the mutation comprises a deletion of nucleotides 4171 through 4198 of SEQ ID NO: 1; (viii) the mutation comprises a deletion of nucleotides 4187 through 4190 of SEQ ID NO: 1; (ix) the mutation comprises a deletion of nucleotides 4189 through 4198 of SEQ ID NO: 1; (x) the mutation comprises a deletion of nucleotides 4120 through 4197 of SEQ ID NO: 1; (xi) the mutation comprises a deletion of nucleotides 4187 through 4191 of SEQ ID NO: 1; (xii) the mutation comprises a deletion of nucleotides 4188 through 4195 of SEQ ID NO: 1; or (xiii) the mutation comprises a deletion of nucleotides 4187 through 4194 of SEQ ID NO: 1.

26-31. (canceled)

32. The method of claim 22, further comprising
(a) introducing editing reagents or a nucleic acid construct encoding said editing reagents into said plant, plant part, or plant cell, or
(b) contacting the plant or plant part with a mutagen, thereby introducing said mutation into said plant or plant part.

33. The method of claim 32:
(a) wherein said editing reagents comprise at least one nuclease and one or more guide RNAs (gRNAs), wherein the nuclease cleaves a target site in a genome of said plant, plant part, or plant cell, and said mutation is introduced at said cleaved target sites or
(b) wherein the mutagen is ethyl methanesulfonate (EMS) or N-ethyl-N-nitrosourea (ENU).

34-45. (canceled)

46. A plant or plant part produced by the method of claim 22, or a population of plants or plant parts comprising the plant or plant part produced by the method of claim 22, wherein said plant, plant part, or population of plants or plant parts comprises reduced beta-amyrin synthase (BAS) activity, decreased saponin content or improved flavor characteristics compared to a control plant or plant part.

47-51. (canceled)

52. A seed, protein, or oil composition or a plant product produced from the or plant part of claim 1, or a population of plants or plant parts of claim 1.

53-56. (canceled)

57. A nucleic acid molecule comprising a nucleic acid sequence of the mutated beta-amyrin synthase (BAS) gene of the plant or plant part of claim 4 :
(a) wherein said nucleic acid sequence has at least 80% identity to a nucleic acid sequence of any one of:
(i) SEQ ID NO: 1 consisting of a deletion of nucleotides 4191 through 4195 thereof;
(ii) SEQ ID NO: 1 consisting of a G to A substitution of nucleotide 3564 thereof;
(iii) SEQ ID NO: 1 consisting of an A to T substitution of nucleotide 374 thereof;
(iv) SEQ ID NO: 1 consisting of a deletion of nucleotides 4190 through 4199 thereof;
(v) SEQ ID NO: 1 consisting of a deletion of nucleotides 4171 through 4198 thereof;
(vi) SEQ ID NO: 1 consisting of a deletion of nucleotides 4187 through 4190 thereof;
(vii) SEQ ID NO: 1 consisting of a deletion of nucleotides 4189 through 4198 thereof;
(viii) SEQ ID NO: 1 consisting of a deletion of nucleotides 4120 through 4197 thereof;
(ix) SEQ ID NO: 1 consisting of a deletion of nucleotides 4187 through 4191 thereof;
(x) SEQ ID NO: 1 consisting of a deletion of nucleotides 4188 through 4195 thereof; or
(xi) SEQ ID NO: 1 consisting of a deletion of nucleotides 4187 through 4194 thereof; or
(b) wherein said nucleic acid sequence comprises the nucleic acid sequence of any one of:
(i) SEQ ID NO: 1 consisting of a deletion of nucleotides 4191 through 4195 thereof;
(ii) SEQ ID NO: 1 consisting of a G to A substitution of nucleotide 3564 thereof;
(iii) SEQ ID NO: 1 consisting of an A to T substitution of nucleotide 374 thereof;
(iv) SEQ ID NO: 1 consisting of a deletion of nucleotides 4190 through 4199 thereof;
(v) SEQ ID NO: 1 consisting of a deletion of nucleotides 4171 through 4198 thereof;
(vi) SEQ ID NO: 1 consisting of a deletion of nucleotides 4187 through 4190 thereof;
(vii) SEQ ID NO: 1 consisting of a deletion of nucleotides 4189 through 4198 thereof;
(viii) SEQ ID NO: 1 consisting of a deletion of nucleotides 4120 through 4197 thereof;
(xi) SEQ ID NO: 1 consisting of a deletion of nucleotides 4187 through 4191 thereof;
(x) SEQ ID NO: 1 consisting of a deletion of nucleotides 4188 through 4195 thereof; or
(xi) SEQ ID NO: 1 consisting of a deletion of nucleotides 4187 through 4194 thereof; or
(c) wherein said nucleic acid sequence encodes a polynucleotide comprising an amino acid sequence:
(i) having at least 80% sequence identity to an amino acid sequence of SEQ ID NO: 6 with a G to E substitution of amino acid 220 or an R to W substitution of amino acid 100; or
(ii) of SEQ ID NO: 6 with a G to E substitution of amino acid 220 or an R to W substitution of amino acid 100, or
(d) wherein said nucleic acid sequence has at least 80% identity to a nucleic acid sequence of:
(i) SEQ ID NO_{:} 38 consisting of a G to A substitution of nucleotide 3750 thereof; or
(ii) SEQ ID NO_{:} 38 consisting of an A to T substitution of nucleotide 560 thereof; or
(e) wherein said nucleic acid sequence comprises the nucleic acid sequence of any one of:
(i) SEQ ID NO_{:} 38 consisting of a G to A substitution of nucleotide 3750 thereof; or
(ii) SEQ ID NO_{:} 38 consisting of an A to T substitution of nucleotide 560 thereof.

58. (canceled)

59. A DNA construct comprising, in operable linkage:
(i) a promoter that is functional in a plant cell; and
(ii) the nucleic acid molecule of claim 57.

60. A cell comprising the nucleic acid molecule of claim 57, or the DNA construct comprising, in operable linkage, a promoter that is functional in a plant cell and the nucleic acid molecule of claim 57.

61. (canceled)

62. A method of producing a population of low-saponin soybean plants or seeds, said method comprising:
a) genotyping a first population of soybean plants or seeds for the presence of at least one low-saponin marker that is within 20 centimorgans of at least one low-saponin quantitative trait locus (QTL) located within a genomic region 132866-141435 of chromosome 7 of a soybean genome;
b) selecting from the first population one or more soybean plants or seeds comprising one or more low-saponin alleles having the one or more low-saponin molecular markers; and
c) producing a second population of progeny soybean plants or seeds from the selected one or more soybean plants or plants grown from the selected seeds,
wherein the second population of progeny soybean plants or seeds comprises the one or more low-saponin alleles having the one or more low-saponin molecular markers, and
wherein the second population of progeny soybean plants or seeds comprises low-saponin content relative to a control population.

63. The method of claim 62:
(a) wherein the at least one low-saponin QTL is Gm07_137242, Gm07_133425, and/or Gm07_136615; or
(b) wherein said at least one low-saponin QTL comprises a single nucleotide polymorphism (SNP), and said at least one low-saponin marker comprises an allele of the SNP; or
(c) wherein said at least one low-saponin QTL comprises a deletion of at least a portion of a beta-amyrin synthase (BAS) gene or regulatory region thereof, and said at least one low-saponin marker comprises an allele comprising the deletion; or
(d) wherein genotyping comprises analyzing the SNP or the deletion using an oligonucleotide probe comprising at least 15 nucleotides, wherein the oligonucleotide probe has at least 90% sequence identity to a sequence of the same number of contiguous nucleotides of a sense or antisense DNA strand in a region comprising or adjacent to the SNP or the deletion; or
(e) wherein the genotyping comprises analyzing the SNP or the deletion using a first primer and a second primer each comprising at least 15 nucleotides, wherein the first primer has at least 90% sequence identity to a sequence of the same number of contiguous nucleotides of a sense DNA strand of a region comprising or adjacent to the SNP, and the second primer has at least 90% sequence identity to a sequence of the same number of contiguous nucleotides of an antisense DNA strand of the region comprising or adjacent to the SNP or the deletion.

64. (canceled)

65. The method of claim 63:
(a) wherein the SNP is a T or an A at position 133425 and/or an A or a G at position 136615 of chromosome 7 of the soybean genome, wherein the T at position 133425 or the A at position 136615 of chromosome 7 of the soybean genome is associated with low-saponin content; or
(b) wherein said deletion comprises a deletion of positions Gm07137242-137246 of the soybean genome; or
(c) wherein the oligonucleotide probe comprises any one of SEQ ID NOs: 17, 18, 21, and 22.

66-75. (canceled)

76. A method of introgressing a low-saponin QTL, the method comprising:
(a) crossing a first soybean plant comprising a low-saponin QTL with a second soybean plant of a different genotype to produce one or more progeny plants or seeds; and
(b) selecting a progeny plant or seed comprising a low-saponin allele of a polymorphic locus linked to the low-saponin QTL,
wherein the polymorphic locus is a chromosomal segment comprising a low-saponin marker within the genomic region 132866-141435 of soybean chromosome 7.

77-83. (canceled)

84. A nucleic acid molecule for detecting a low-saponin molecular marker in soybean DNA, the nucleic acid molecule comprising at least 15 nucleotides, wherein the nucleic acid molecule has at least 90% sequence identity to a sequence of the same number of contiguous nucleotides of a sense or antisense DNA strand in a region comprising or adjacent to the low-saponin molecular marker:
(a) wherein the low-saponin molecular marker is an SNP marker, and wherein the SNP marker is a T or an A at position 133425 or an A or a G at position 136615 of chromosome 7 of the soybean genome, wherein the T at position 133425 or the A at position 136615 of chromosome 7 of the soybean genome is associated with low-saponin content; or
(b) wherein the low-saponin molecular marker is a deletion marker, and wherein the deletion maker is a deletion of positions Gm07137242-137246; or
(c) wherein said nucleic acid molecule comprises any one of SEQ ID NOs: 17, 18, 21, and 22; or
(d) wherein the nucleic acid molecule further comprises a detectable label.

85-89. (canceled)
